(19) United States
(12) Patent Application Publication
(10) Pub. No.: US 2009/0221581 A1
(43) Pub. Date: Sep. 3, 2009

(54) Methods of treating pain

(76) Inventors: Philipp Wabnitz, Düsseldorf (DE); Heike Schauerte, München (DE); Gabriele Stumm, Unteraching (DE); Joachim Freitag, Nürtingen (DE)

Correspondence Address: Olson & Cepuritis, LTD., 20 NORTH WACKER DRIVE, 36TH FLOOR, CHICAGO, IL, 60606, US

(21) Appl. No.: 11/920,883

(22) PCT Filed: May 24, 2006
(86) PCT No.: PCT/EP2006/004924

§ 371 Date: Jan. 18, 2009

Related U.S. Application Data
(60) Provisional application No. 60/684,345, filed on May 25, 2005.

(51) Int. Cl. A61K 31/506; A61K 31/5377; A61P 29/00

(52) U.S. Cl. 514/235.8; 514/275

(57) ABSTRACT

The invention relates to methods of treating any type of pain comprising the administration of an effective amount of at least one inhibitor of cyclin-dependent kinases.

### FIELD OF THE INVENTION

The present invention relates to methods of treating any type of pain comprising the administration of an effective amount of at least one inhibitor of cyclin-dependent kinases.

### BACKGROUND OF THE INVENTION

Debilitating acute or chronic pain is a constant backdrop to daily life for many people. Current estimates suggest that 1 in 10 adults suffer from chronic pain at some point in their lives. In terms of both lost productivity and treatment, the cost to society in the US alone surpasses 100 billion dollars annually.

Unfortunately, current treatments for pain are only partially effective, and many also cause debilitating or dangerous side effects. For example, many of the traditional analgesics used to treat severe pain induce debilitating side effects such as nausea, dizziness, constipation, respiratory depression, and cognitive dysfunction (Brower, 2000).

Although there is already a broad panel of approved pain medications like non-narcotic analgesics, opioid analgesics, calcium channel blockers, muscle relaxants, and systemic corticosteroids available, said treatments remain merely empirical and, while they may relieve the symptoms of pain, they do not lead to complete relief in most cases. This is also due to fact that the mechanisms underlying the development of the different types of pain are still only poorly understood. Researchers are only just beginning to appreciate the complexity and diversity of the signaling systems used to relay nerve impulses for each type of pain.

Generally, pain is defined as an unpleasant sensory and emotional experience associated with actual or potential tissue damage, or described in terms of such damage, according to the International Association for the Study of Pain (IASP). Specifically, pain may occur as acute or chronic pain.

Acute pain occurs for brief periods of time, typically less than 1 month and is associated with temporary disorders. It is a natural body response to let the host be aware of physiological or biochemical alteration that could result in further damage within a short period of time. It is felt when noxious stimuli activate high threshold mechanical and/or thermal nociceptors in peripheral nerve endings and the evoked action potentials in thinly myelinated (Aδ) and/or unmyelinated (C) afferent fibres reach a conscious brain. Said noxious stimuli may be provided by injury, surgery, illness, trauma or painful medical procedures. Acute pain usually disappears when the underlying cause has been treated or has healed. Unrelieved acute pain, however, may lead to chronic pain problems that may result in long hospital stays, rehospitalizations, visits to outpatient clinics and emergency departments, and increased health care costs.

In contrast to acute pain, chronic pain persists long after the initial injury has healed and often spreads to other parts of the body, with diverse pathological and psychiatric consequences. Chronic somatic pain results from inflammatory responses to trauma in peripheral tissues (e.g., nerve entrapment, surgical procedures, cancer, or arthritis), which leads to oversensitization of nociceptors and intense searing pain responses to normally non-noxious stimuli (hyperalgesia). Chronic pain is continuous and recurrent and its intensity will vary from mild to severe disabling pain that may significantly reduce quality of life.

Chronic pain is currently treated with conventional analgesics such as Non-steroidal anti-inflammatory drugs (NSAIDs such as Ibuprofen, Naproxen), Cox-2 inhibitors (Celecoxib, Valdecoxib, Rofecoxib) and opiates (codeine, morphin, thebain, papaverin, noscapin). For a significant number of patients however, these drugs provide insufficient pain relief.

Another subtype of pain, inflammatory pain, can occur as acute as well as chronic pain. Resulting injuries of tissue and neurons must not but may develop into long-lasting chronic neuropathic pain effects in succession to such inflammatory events.

Inflammatory pain is mediated by noxious stimuli like e.g. inflammatory mediators (e.g. cytokines, such as TNFα, prostaglandins, substance P, bradykinin, purines, histamine, and serotonine), which are released following tissue injury, disease, or inflammation and other noxious stimuli (e.g. thermal, mechanical, or chemical stimuli). In addition, cytokines and growth factors can influence neuronal phenotype and function (Besson 1999). These mediators are detected by nociceptors (sensory receptors) that are distributed throughout the periphery of the tissue. Said nociceptors are sensitive to noxious stimuli (e.g. mechanical, thermal, or chemical), which would damage tissue if prolonged (Koltzenburg 2000). A particular class of so called C-nociceptors represent a class of “silent” nociceptors that do not respond to any level of mechanical or thermal stimuli but are activated in presence of inflammation only.

Current approaches for the treatment of especially inflammatory pain aim at cytokine inhibition (e.g. IL1β) and suppression of pro-inflammatory TNFα. Current approved anticytokine/antiTNFα treatments are based on chimeric antibodies such as Infliximab and Etanercept which reduce TNFα circulation in the bloodstream. TNFα is one of the most important inflammatory mediators which induces synthesis of important enzymes such as COX-2, MMP, iNOS, cPLa₂ and others. The main drawbacks of “biologicals” such as chimeric antibodies, however, reside in their immunogenic potential with attendant loss of efficacy and their kinetics, leading to a more or less digital all-or-nothing reduction of circulating TNFα. The latter can result in severe immune suppressive side effects.

A distinct form of chronic pain, neuropathic (or neurogenic) pain, arises as a result of peripheral or central nerve dysfunction and includes a variety of conditions that differ in aetiology as well as location. Generally, the causes of neuropathic pain are diverse, but share the common symptom of damage to the peripheral nerves or components of central pathways. The causative factors might be metabolic, viral or a mechanical nerve lesion. Neuropathic pain is believed to be sustained by aberrant somatosensory processes in the peripheral nervous system, the CNS, or both. Neuropathic pain is not directly linked to stimulation of nociceptors, but instead, is thought to arise e.g. from oversensitization of glutamate receptors on postsynaptic neurons in the gray matter (dorsal horn) of the spinal cord.

Neuropathic pain is associated with conditions such as nerve degeneration in diabetes and postherpetic neuralgia (shingles). Neuropathic pain conditions are the consequence of a number of diseases and conditions, including diabetes, AIDS, multiple sclerosis, stump and phantom pain after amputation, cancer-related neuropathy, post-herpetic neuralgia, traumatic nerve injury, ischemic neuropathy, nerve compression, stroke and spinal cord injury.

Management of neuropathic pain remains a major clinical challenge, partly due to an inadequate understanding of the mechanisms involved in the development and maintenance of neuropathic pain. Many existing analgesics are ineffective in treating neuropathic pain and most of current narcotic and non-narcotic drugs do not provide control of neuropathic pain. Current clinical practice includes the use of a number of drug classes for the management of neuropathic pain, for example anticonvulsants, tricyclic antidepressants, and systemic local anaesthetics. However, the usual outcome of such treatment is merely partial or unsatisfactory pain relief, and in some cases the adverse effects of these drugs outweigh their clinical usefulness. Classic analgesics are widely believed to be poorly effective or ineffective in the treatment of neuropathic pain. Few clinical studies on the use of non steroidal anti-inflammatory drugs (NSAIDs) or opiates in the treatment of neuropathic pain have been conducted, but in those which have, the results appear to indicate that NSAIDs are poorly effective or ineffective and opiates only work at high doses. A review analysing the controlled clinical data for peripheral neuropathic pain (PNP) (Pain 1997 73(2), 123-39) reported that NSAIDs were probably ineffective as analgesics for PNP and that there was no long-term data supporting the analgesic effectiveness of any drug.

Summarizing, available analgesic drugs often only produce insufficient pain relief. Although tricyclic antidepressants and some antiepileptic drugs, for example gabapentin, lamotrigine and carbamazepine, are efficient in some patients, there remains a large unmet need for efficient drugs for the treatment of these conditions.

In conclusion, there is a high unmet need for safe and effective methods of treating any type of pain, in particular chronic inflammatory and neuropathic pain.

### SUMMARY OF THE INVENTION

The present invention is directed to methods and compositions for treating any type of pain, comprising: administering an effective amount of at least one inhibitor of a cyclin-dependent kinase (CDK) to a subject in need thereof. In a preferred embodiment, the inhibitor is selected from the class of 4,6-disubstituted aminopyrimidine derivatives as disclosed in WO2005/026129 and specifically inhibits CDK9. Furthermore, the present invention specifically relates to methods of treating neuropathic and/or inflammatory pain.

### BRIEF DESCRIPTION OF THE DRAWINGS

FIG. 1 schematically depicts the spared nerve injury model (SNI model, as developed by Decosterd and Woolf (2000), which is characterized by ligation and section of two branches of the sciatic nerve (namely tibial and common peroneal nerves) leaving the sural nerve intact.

FIG. 2 schematically depicts a possible role of CDK9 as a target in the development of pain.

FIG. 3A depicts the results of von Frey measurements performed with SNI mice which had been treated with compound A. The observations are indicative for reduced allodynia/reduced neuropathic pain perception in CDK9 inhibitor-treated mice and for a hypoalgesic effect of CDK9 inhibitor compound A.

FIG. 3B depicts the average values of results of von Frey measurements performed with SNI mice which had been treated with compound A. The observations are indicative for reduced allodynia/reduced neuropathic pain perception in CDK9 inhibitor-treated mice and for a hypoalgesic effect of CDK9 inhibitor compound A.

FIG. 4 displays the averages of licking time observed in formalin-treated mice (n=10) treated with compound B (30 mg/kg) or vehicle (manual analysis). The observation is indicative for reduced inflammatory/chronic inflammatory pain perception in CDK9 inhibitor-treated mice and for a hypoalgesic effect of CDK9 inhibitor compound B.

FIG. 5 displays the averages of licking time observed in formalin-treated mice (n=10) treated with compound A (30 mg/kg), IKK inhibitor (30 mg/kg, positive control), vehicle (DMA/Labrafil, negative control), or no treatment (none), respectively, all analyzed by manual observation. The observation is indicative for reduced inflammatory/chronic inflammatory pain perception in CDK9 inhibitor-treated mice and for a hypoalgesic effect of CDK9 inhibitor compound A.

FIG. 6 depicts the course of developing hind leg paw edema over a time course of 98 h after carrageenan injection into mice hind paws. Mice were partially treated with CDK9 inhibitor compound A and respective controls (Vehicle, Saline) thereof. The observations are indicative for reduced inflammation in CDK9 inhibitor (compound A)-treated mice.

FIG. 7 depicts suppression of TNFα-expression in microglial cells after treatment with compound A.

FIG. 8 depicts effects of intraperitoneal treatment of mice with 30 mg/kg compound B 30 minutes prior to carrageenan application. The observations are indicative of a significant reduction of thermal hyperalgesia detected by the Hargreaves assay comparable to 50 mg/kg Naproxen.

FIG. 9 depicts the results of von Frey measurements performed with SNI mice which had been treated i.p. with 30 mg/kg IPAG11 30 minutes before von Frey testing. The observations are indicative of a significant reduction of SNI-induced allodynia.

### DETAILED DESCRIPTION OF THE INVENTION

The invention relates to methods and compositions for treating any type of pain, comprising: administering an effective amount of at least one inhibitor of a cyclin-dependent kinase (CDK) to a subject in need thereof.

The term “pain” as used herein generally relates to any type of pain and broadly encompasses types of pain such as acute pain, chronic pain, inflammatory and neuropathic pain.

In a preferred embodiment of the present invention, pain comprises neuropathic pain and associated conditions. The pain may be chronic, allodynia (the perception of pain from a normally innocuous stimulus), hyperalgesia (an exaggerated response to any given pain stimulus) and an expansion of the receptive field (i.e. the area that is “painful” when a stimulus is applied), phantom pain or inflammatory pain.

Acute pain types comprise, but are not limited to pain associated with tissue damage, postoperative pain, pain after trauma, pain caused by burns, pain caused by local or systemic infection, visceral pain associated with diseases comprising: pancreatitis, intestinal cystitis, dysmenoirhea, Irritable Bowel syndrome, Crohn's disease, ureteral colic and myocardial infarction.

Furthermore, the term “pain” comprises pain associated with CNS disorders comprising: multiple sclerosis, spinal cord injury, traumatic brain injury, parkinson's disease and stroke.

In a preferred embodiment, “pain” relates to chronic pain types comprising headache (for example migraine disorders, episodic and chronic tension-type headache, tension-type like headache, cluster headache, and chronic paroxysmal hemicrania), low back pain, cancer pain, osteoarthritis pain and neuropathic pain, but is not limited thereto.

Inflammatory pain (pain in response to tissue injury and the resulting inflammatory process) as defined herein relates to inflammatory pain associated with diseases comprising connective tissue diseases, rheumatoid arthritis, systemic lupus erythematosus, multiple sclerosis and arthritis, but is not limited thereto.

Specifically, neuropathic pain (pain resulting from damage to the peripheral nerves or to the central nervous system itself) may comprise painful diabetic peripheral neuropathy, post-herpetic neuralgia, trigeminal neuralgia, cranial neuralgia, post-stroke neuropathic pain, multiple sclerosis-associated neuropathic pain, post-surgical neuropathic pain, neuropathy-associated pain such as in idiopathic or post-traumatic neuropathy and mononeuritis, HIV/AIDS-associated neuropathic pain, cancer-associated neuropathic pain, carpal tunnel-associated neuropathic pain, spinal cord injury-associated pain, complex regional pain syndrome, fibromyalgia-associated neuropathic pain, lumbar and cervical pain, reflex sympathic dystrophy, phantom limb syndrome or peripheral nerve or spinal cord trauma, entrapment neuropathy, nerve transection including surgery, Lissauer tract section, limb amputation and stump pain, neuroma/tumour compression, arteriovenous malformation, Vitamin B12 deficiency, diabetic neuropathy, alcoholic neuropathy, pain caused by the side effects of anti-cancer and anti-AIDS therapies, pain associated with inflammation or infection of a tooth (toothache), visceral pain, pain caused by chemical burns, pain caused by local or systemic infection, or pain caused by connective tissue disease. The connective tissue disease may be one of: rheumatoid arthritis, Wallenberg's syndrome, systemic lupus erythematosus, multiple sclerosis, or polyarteritis nodosa. The neuropathy can be classified as radiculopathy, mononeuropathy, mononeuropathy multiplex, polyneuropathy or plexopathy. Diseases in this class can be caused by a variety of nerve-damaging conditions or procedures, including, without limitation, trauma, stroke, demyelinating diseases, abscess, surgery, amputation, inflammatory diseases of the nerves, causalgia, diabetes, collagen vascular diseases, trigeminal neuralgia, rheumatoid arthritis, toxins, cancer, chronic alcoholism, herpes infection, AIDS and chemotherapy. Nerve damage causing hyperalgesia can be in peripheral or CNS nerves.

The term “allodynia” denotes pain arising from stimuli which are normally not painful.

Allodynic pain may occur other than in the area stimulated

The terms “hyperalgesia”/“hyperalgesic” denote an increased sensitivity to a painful stimulus.

The terms “hypoalgesia”/“hypoalgesic” denote a decreased sensitivity to a painful stimulus.

The present invention relates to a method for treating any type of pain, such as the above-referenced types of pain and associated conditions, wherein the term “treating” comprises the prevention, amelioration or treatment of any type of pain and associated conditions.

Specifically, the invention relates to a method for the treatment of neuropathic and/or inflammatory pain, comprising: administering an effective amount of at least one inhibitor of a cyclin-dependent kinase (CDK) to a subject in need thereof.

Cyclin-dependent protein kinases (“CDKs”), constitute a family of well-conserved enzymes that play multiple roles within the cell, such as cell cycle regulation and transcriptional control (Science, vol. 274, pp. 1643-1677 (1996); and Ann. Rev. Cell Dev. Biol., vol. 13, pp. 261-291 (1997).

Some members of the family, such as CDK1, 2, 3, 4, and 6 regulate the transition between different phases of the cell cycle, such as the progression from a quiescent stage in G1 (the gap between mitosis and the onset of DNA replication for a new round of cell division) to S (the period of active DNA synthesis), or the progression from G2 to M phase, in which active mitosis and cell division occur. Other members of this group of proteins, including CDK7, 8, and 9 regulate key points in the transcription cycle.

CDK complexes are formed through association of a regulatory cyclin subunit (e.g., cyclin A, B1, B2, D1, D2, D3, and E) and a catalytic kinase subunit (e.g., cdc2 (CDK1), CDK2, CDK4, CDK5, and CDK6). As the name implies, the CDKs display an absolute dependence on the cyclin subunit in order to phosphorylate their target substrates, and different kinase/cyclin pairs function to regulate progression through specific portions of the cell cycle.

CDK9 in association with its cyclin partners (cyclin T1, T2a, T2b, or K) constitutes the catalytic component of the positive P-TEFb protein kinase complex that functions during the elongation phase of transcription by phosphorylating the carboxyl-terminal domain (CTD) of the largest subunit of RNA polymerase II. P-TEFb acts in concert with positive transcription factor NfκB as well as negative transcription factors, thus overcoming a block of transcriptional elongation (Liu and Herrmann, J. Cell Physiol, 2005).

One of the key discoveries underlying the present invention is the demonstration that administration of CDK inhibitors to mice suffering from nerve lesion exerts a hypoalgesic effect, in particular in murine models of inflammatory and neuropathic pain.

The discovery that inhibition of a cyclin-dependent kinase is involved in mediating a hypoalgesic effect was unexpected.

In a preferred embodiment of this invention, the cyclin-dependent kinase inhibitor inhibits a CDK selected from the group consisting of CDK1, CDK2, CDK3, CDK4, CDK5, CDK6, CDK7, CDK8, CDK9, CDK10, CDK11, CrkRS (Crk7, CDC2-related protein kinase 7), CDKL1 (cyclin-dependent kinase-like 1); KKIALRE, CDKL2 (cyclin-dependent kinase-like 2), KKIAMRE, CDKL3 (cyclin-dependent kinase-like 3), NKIAMRE, CDKL4, similar to cyclin-dependent kinase-like 1, CDC2L1 (cell division cycle 2-like 1), PITSLRE B, CDC2L1 (cell division cycle 2-like 1), PITSLRE A, CDC2L5 (cell division cycle 2-like 5), PCTK1 (PCTAIRE protein kinase 1), PCTK2 (PCTAIRE protein kinase 2), PCTK3 (PCTAIRE protein kinase 3) or PFTK1 (PFTAIRE protein kinase 1).

The inhibitor may also inhibit more than one cyclin-dependent kinase selected from the above-recited group.

In a preferred embodiment of this invention, the CDK inhibitor inhibits CDK9. In a particular preferred embodiment, the CDK inhibitor is a selective inhibitor of CDK9.

The role of CDK9 in the development of pain could be based on the following mechanism of action: Both cyclin Ti and CDK9 stimulate the basal promoter activity of TNFα. TNFα is a pro-inflammatory cytokine and pain mediator that controls expression of inflammatory genetic networks. For mediation of cellular TNF receptor responses, the nuclear factor-KB (NFκB) pathway is crucial. TNFα triggers its recruitment to cytokine genes while NfκB interacts with the p-TEFb complex for stimulation of gene transcription (Barboric M et al., 2001).

Additionally, it has been shown that CDK9 is a binding partner of TRAF2, a member of the TNFα receptor complex (MacLachlan et al, 1998), while GP130, a subunit of the pro-inflammatory IL6 receptor complex has recently been identified as another potential binding partner of CDK9 (Falco et al, 2002). As a key player in TNFα and interleukin signaling as well as NfκB mediated expression of several genes (e.g. cytokines as pain mediators), CDK9 can thus be considered as a central target for the treatment of inflammatory pain (see FIG. 2).

For the treatment of neuropathic pain, pharmacological action has to take place beyond blood-brain-barrier (BBB) in the central nervous system (CNS). Microglial cells as the principal immune cells in the CNS, release, if activated, a variety of noxious factors such as cytokines (TNFα, IL1β, IL6) and other pro-inflammatory molecules (Huwe, 2003). Microglia is activated by stimulation of TNFα receptor or Toll-like receptor and signal is mediated via Iκ kinase (IKK) and NfκB leading to transcriptional activation of the cytokines described above. Microglial contribution has been discussed as instrumental in chronic CNS diseases and may contribute to pain perception (Watkins et al, 2003).

Recently it has been shown that NfκB regulates expression of Cyclooxygenase-2 (COX-2) via Interleukin Iβ (IL1β) in the spinal cord (Lee et al., 2004). As the major contributor to elevation of spinal prostaglandin E2, the pain mediator COX-2 is already known as a target for a variety of anti-nociceptive/anti-inflammatory drugs. NfκB inhibitors have proven their ability to reduce COX-2 levels and mechanical allodynia as well as thermal hyperalgesia in animal models significantly.

In contrast to inhibition of Cox-2, inhibition of CDK9 action would lead to suppression of a variety of pain mediators instead of just a single one. Thereby, anti-nociceptive action of CDK9 inhibitors might be improved in comparison to e.g. COX-2 inhibitors.

Due to its relevance for NfκB mediated gene transcription, inhibition of CDK9 may therefore be a reasonable approach not only for the treatment of acute inflammatory pain, but also for the treatment of chronic pain.

There are many known inhibitors of CDKs that may be used in the methods and compositions of the present invention.

The terms “cyclin-dependent kinase inhibitor”/“cyclin-dependent kinase inhibitory compound”” refer to any compound or group of compounds capable of downregulating, decreasing, suppressing or otherwise regulating the amount and/or activity of a cyclin-dependent kinase. Inhibition of said kinases can be achieved by any of a variety of mechanisms known in the art, including, but not limited to binding directly to the kinase polypeptide, denaturing or otherwise inactivating the kinase, or inhibiting the expression of the gene (e.g., transcription to mRNA, translation to a nascent polypeptide, and/or final polypeptide modifications to a mature protein), which encodes the kinase. Furthermore, a cyclin-dependent kinase inhibitor may also interfere with expression, modification, regulation or activation of a molecule acting downstream of a CDK in a CDK-dependent pathway. Generally, kinase inhibitors may be proteins, polypeptides, nucleic acids, small molecules, or other chemical moieties. Specifically, kinase inhibitors also include monoclonal or polyclonal antibodies directed against cyclin-dependent kinases.

As used herein, the term “inhibiting” or “inhibition” refers to the ability of a compound to downregulate, decrease, reduce, suppress, inactivate, or inhibit at least partially the cellular function of a cyclin-dependent kinase, i.e. its activity or the expression of the cyclin-dependent kinase.

While any compound that inhibits a CDK is considered suitable to treat any type of pain in the context of the present invention, compounds that selectively inhibit one or more CDKs without having a substantial inhibitory effect on other enzymes or proteins are preferred for treating pain, preventing future pain and/or inhibiting increased sensitivity to a noxious stimulus.

In a preferred embodiment, such inhibitory compounds display an increased selectivity for a particular CDK, i.e. they are able to selectively inhibit a particular CDK. “Increased selectivity”/“ability to selectively inhibit a particular CDK” as used herein means that the inhibitory compound is 10-100× more selective for a particular CDK selected from the group of CDKs as recited herein, supra. In a preferred embodiment of the present invention, the inhibitory compound is 20-90× more selective for a particular CDK. In a particular preferred embodiment, the inhibitory compound is 30-80× more selective for a particular CDK.

In a particular preferred embodiment, the CDK inhibitor displays an increased selectivity for CDK9 than for other CDKs.

For example, the inhibitory compound may be 30-80× more selective for inhibiting CDK9 than for inhibiting other CDKs.

Compounds which display an increased selectivity for inhibiting a particular CDK than for inhibiting other CDKs are denoted “selective inhibitors” of the respective CDK.

Cyclin-dependent kinase inhibitors that are useful in the methods and compositions of the present invention include low-molecular-weight inhibitors such as staurosporin, flavones, butyrolactone I, purines, pyrimidines, pyrido[2,3-d]pyrimidones, oxoindoles, paullones, fascaplysin, hymenialdisine, acridones and benzothiadiazines and diarylurea derivatives.

Olomoucine and roscovitine are examples for purine CDK inhibitors. Furthermore, the group of flavones is exemplified by Flavopiridol, which is described as an unselective pan-inhibitor of CDKs, including CDK9 (W. Filgueira de Azevedo et al., Biochem. and Biophys. Res. Commun. 2002, 293(1), 566-571). Flavopiridol binds to its target with a higher affinity than another widely used CDK9 inhibitor, namely 5,6-dichloro-1-beta-D-ribofuranosylbenzimidazole (DRB) (Chao and Price, 2001). The use of flavopiridol as Cdk9 modulator for the treatment of cardiovascular diseases has been described in WO 2005/027092.

So far, many of these inhibitory compounds are merely known for their anti-tumorigenic action via the inhibition of CDK1 and CDK2 (Current Opinion in Pharmacology, 2003, 3, 1-9). For a review of known cyclin-dependent kinase inhibitors, see Huwe et al. 2003; Dai and Grant, 2003.

In a preferred embodiment of this invention, the cyclin-dependent kinase inhibitor used in the methods and compositions of the present invention is selected from the class of pharmaceutically active 4,6-disubstituted aminopyrimidine derivatives as disclosed in WO 2005/026129, which is incorporated herein in entirety. Preferably, said 4,6-disubstituted aminopyrimidine derivatives inhibit CDK9.

In a particular preferred embodiment, said 4,6-disubstituted aminopyrimidine derivatives display an increased selectivity for CDK9 than for other CDKs.

Said 4,6-disubstituted aminopyrimidine derivatives are defined in WO2005/026129 by the general formula (I)

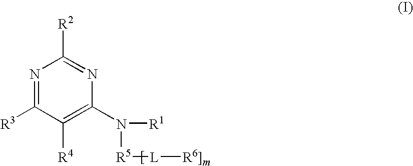

wherein R¹ is selected from the group comprising: —H, linear or branched C₁-C₆ substituted or unsubstituted alkyl, linear or branched C₂-C₆ alkenyl or linear or branched C₂-C₆ alkinyl;

R² and R⁴ are independently selected from the group consisting of: —H, linear or branched C₁-C₆ substituted or unsubstituted alkyl, linear or branched C₂-C₆ alkenyl, linear or branched C₂-C₆ alkinyl, aryl, —F, —Cl, —Br, —I, —CN, —NH₂ or —NO₂;

R³ is selected from the group comprising: —F, —Cl, —Br, —I, substituted or unsubstituted aryl, substituted or unsubstituted —O-aryl, —NH-aryl, —S-aryl, or substituted or unsubstituted —O-heterocyclyl, —NH-heterocyclyl, —S-heterocyclyl, or substituted or unsubstituted —CH═CH-aryl, or substituted or unsubstituted heteroaryl, or substituted or unsubstituted heterocyclyl, or substituted or unsubstituted C₃-C₈ cycloalkyl, or —NH—(CH₂)ₙ—X, wherein n is an integer from 0 to 6 and X is selected from —OH, —NH₂ or substituted or unsubstituted C₃-C₈ cycloalkyl;

R⁵ is selected from the group consisting of: substituted or unsubstituted aryl, substituted or unsubstituted heteroaryl, substituted or unsubstituted heterocyclyl, substituted or unsubstituted C₃-C₈ cycloalkyl, or —(CH₂)ₒ—Y, wherein o is an integer from 0 to 6 and Y represents substituted or unsubstituted aryl, substituted or unsubstituted heteroaryl, substituted or unsubstituted heterocyclyl or substituted or unsubstituted C₃-C₈ cycloalkyl;

R⁶ is selected from the group consisting of: —H, linear or branched substituted or unsubstituted C₁-C₈ alkyl, substituted or unsubstituted aryl, substituted or unsubstituted heteroaryl, substituted or unsubstituted heterocyclyl, substituted pyrrolidinyl, substituted or unsubstituted C₃-C₈ cycloalkyl, disubstituted cyclohexyl, cyclopentyl, substituted or unsubstituted C₅-C₁₂ bicycloalkyl, substituted or unsubstituted adamantyl, —(CH₂)_{q}-group, wherein q is an integer from 1 to 3, under the proviso, if R⁶ is selected to be a methylene chain —(CH₂)_{q}-group, R¹⁷ or R¹⁹ are selected to be a methylene chain —(CH₂)ₛ-group, wherein s is an integer from 1 to 3 or a —(CH₂)ₜ-A-group, t is an integer from 1 to 3 and A is selected from O or N, respectively, and R⁶ and R¹⁷ or R⁶ and R¹⁹ form together a 5 to 8 membered ring system, or R⁶ represents —(CH₂)ₚ-Z, wherein p is an integer from 0 to 6 and Z is selected from the group comprising: substituted or unsubstituted aryl, substituted or unsubstituted heteroaryl, substituted or unsubstituted heterocyclyl, —N(R⁷R⁸), wherein R⁷ and R⁸ represent independently from each other —H, or linear or branched substituted or unsubstituted C₁-C₆ alkyl, or Z is selected from —(CR⁹R¹⁰R¹¹), wherein R⁹, R¹⁰ and R¹¹ are independently of each other selected from the group consisting of: —H, linear or branched substituted or unsubstituted C₁-C₈ alkyl, substituted or unsubstituted aryl or —N(R¹²R¹³), wherein R¹² and R¹³ represent independently of each other —H or linear or branched substituted or unsubstituted C₁-C₆ alkyl, under the proviso, if Z represents —(CR⁹R¹⁰R¹¹) as defined above, p is selected to be an integer from 0 to 6, and if Z is selected from substituted or unsubstituted aryl, substituted or unsubstituted heteroaryl, substituted or unsubstituted heterocyclyl, or —N(R⁷R⁸) as defined above, p is selected to be an integer from 1 to 6;

L is selected from the group comprising:

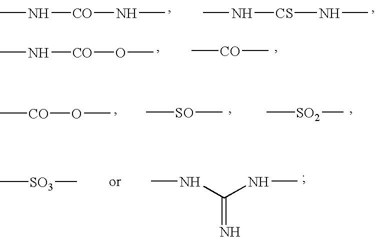

—NR¹⁴—SO₂—, —NR¹⁴—SO—, wherein R¹⁴ is selected from —H, linear or branched substituted or unsubstituted C₁-C₆ alkyl, —SO₂—R¹⁵, wherein R¹⁵ is selected from linear or branched C₁-C₆ alkyl, or R¹⁴ represents —(CH₂), —COOR⁶, wherein r is an integer from 0 to 6 and R¹⁶ is selected from —H or linear or branched substituted or unsubstituted C₁-C₆ alkyl,

—NR¹⁷—CO—, wherein R¹⁷ is selected from —H, linear or branched substituted or unsubstituted C₁-C₆ alkyl, or a-(CH₂)ₛ-group, wherein s is an integer from 1 to 3, and wherein R⁶ and R¹⁷ represent both a methylene chain group, R⁶ and R¹⁷ may form together a 5 to 8 membered ring system:

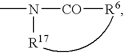

—SO₂—NR¹⁸—, wherein R¹⁸ is selected from —H, or linear or branched substituted or unsubstituted C₁-C₆ alkyl,

—CO—NR¹⁹—, wherein R¹⁹ is selected from —H, linear or branched substituted or unsubstituted C₁-C₆ alkyl, or a —(CH₂)ₜ-A-group, wherein t is an integer from 1 to 3 and A is selected from N or O, and wherein if R⁶ represents a —(CH₂)_{q}-group and R¹⁹ represents a-(CH₂)ₜ-A-group,

R⁶ and R¹⁹ may form together a 5 to 8 membered ring system

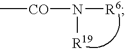

and m is selected to be 0 or 1, and/or stereoisomeric forms and/or pharmaceutical acceptable salts thereof.

In the following, any reference to “general formula (I)” refers to general formula (I) as disclosed in WO2005/026129.

Preferred are compounds having the general formula (I):

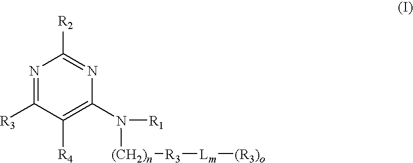

wherein

each R₁ represents independently R₃, R₅, —H, linear or branched substituted or unsubstituted C₁-C₆ alkyl, linear or branched C₂-C₆ alkenyl or linear or branched C₂-C₆ alkinyl or adamantyl,

R₂ and R₄ are independently selected from the group consisting of: R₃, R₅, —H, —CN, —NH₂, —NO₂, linear or branched substituted or unsubstituted C₁-C₆ alkyl, linear or branched C₂-C₆ alkenyl or C₂-C₆ linear or branched alkinyl;

R₃ and R₃′ are independently selected from the group consisting of:

a) halogen, represented by —F, —Cl, —Br or —I,
b) C₃-C₈ cycloalkyl, which is optionally substituted by at least one of the groups R₆, R₆′, R₇ or R₇′,
c) C₄-C₁₂ bicyclo-alkyl, which is optionally substituted by at least one of the groups R₆, R₆′, R₇ or R₇′,
d) aryl, which is optionally substituted by at least one of the groups R₆, R₆′, R₇ or R₇′,
e) X-aryl, which is optionally substituted by at least one of the groups R₆, R₆′, R₇ or R₇′ and wherein X is independently selected from —O—, —NH—, —S—, linear or branched-CH₂— (C₂-C₆ alkyl)-group, linear or branched —CH₂—(C₂-C₆ alkenyl)-group, which is optionally substituted by at least one of the groups R₆, R₆′, R₇ or R₇′,
f) partially or fully saturated 5 or 6 membered heterocyclic ring, which is optionally substituted by at least one of the groups R₆, R₆′, R₇ or R₇′; this heterocyclic ring can be fused to another 5 or 6 membered heterocyclic ring, which is optionally substituted by at least one of the groups R₆, R₆′, R₇ or R₇′, or a 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic group, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′;
g) guanidinyl group, optionally substituted by at least one group R₅ or
h) —Y—(CH₂)ₚ-Z-group, wherein Y represents O, S or NR₅ and Z represents R₅, —OR₅, —N(R₅)₂ or —COOR₅,
wherein in the cases, that the group R₃′ represents one of the groups cited under a), g) or h) the indices m and o of the -(L)ₘ-(R₅)ₒ-group are selected to be 0,

R₅ is independently selected from the group consisting of: —H, R₁, R₂, R₃, R₄, —(CH₂)_{q}—COOR₁, —CH═CH—COOR₁, —C(R₁)₂N(R₁)₂, —(CH₂)ᵣN(R₁)₂, —NR₁—COOR₁ or —C(R₁)₃,

R₆ and R₆′ are independently selected from the group consisting of: R₁, R₂, R₄, R₅, L-H, —H, —OR₁, —N(R₁)₂, —C(R₁)₃, —CH(R₁)₂, or —CH₂R₁;

R₇ and R₇′ represent independently from each other R₆ and R₆′;

L is selected from the group comprising: —NR₅—SO₂—, —NR₅—CO—(CH₂)ₛ—, —NH—CO—NH—, —CO—NR₅—, —SO₂—NR₅— or —NH— under the proviso, that if m is selected to be 1, o is selected to be 1 as well,

m is independently selected to be 0 or 1

n is independently selected to be an integer from 0 to 6,

o is independently selected to be 0 or 1,

p, q, r and s are independently from each other an integer from 0 to 6 and/or stereoisomeric forms and/or pharmaceutically acceptable salts thereof.

In formula (I) shown above, the group R₃′-Lₘ-(R₅)ₒ is to be understood in the sense, that the group denoted by R₃′ is optionally substituted by a group -Lₘ-(R₅)ₒ. This means that if R₃′ is for instance an aryl group, such as phenyl, one of the hydrogen atoms bonded to the aryl group is exchanged by a -Lₘ-(R₅), group.

The group aryl as used in items d) and e) of the definition of the groups R₃ and R₃′, preferably describes an aryl group independently selected from the group consisting of phenyl, biphenyl or napthyl.

In a preferred embodiment of the compounds according to formula (I), the rings defined under f) of the definition of the groups R₃ and R₃′ are independently selected to be

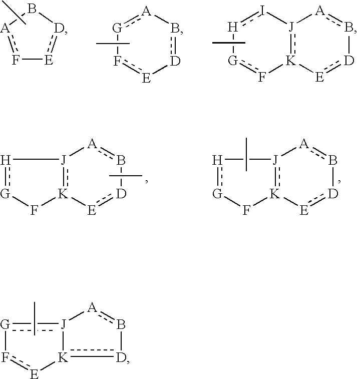

wherein
A, B, D, E, F, G, H and I represent independently of each other: CR₆, C(R₆)₂, N, NR₆, O or SR₆,

J and K are independently from each other: C or N, under the proviso that 0-O and S—S bonds are excluded and that at least one of the ring atoms in the heterocycle is N, S or O, and each [symbol] represent independently from each other a single or a double bond under the proviso that one of the groups R₆ comprised in A, B, D, E, F, G, H, I, J and K is exchanged with a -(L)ₘ-(R₅)ₒ-group.

In a further preferred embodiment of the compounds according to the invention R₁, R₂ and R₄ represent independently of each other R₃, R₅, —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH₂—C(CH₃)₃, —CH(CH₃)—C₃H₇, —CH₂—CH(CH₃)—C₂Hs, —CH(CH₃)—CH(CH₃)₂, —C(CH₃)₂—C₂H₅, —CH₂—C(CH₃)₃, —C₂H₄—CH(CH₃)₂, —C₆H₁₃, —C₃H₆—CH(CH₃)₂, —C₂H₄—CH(CH₃)—C₂Hs, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —C≡CH, —CH₂—CH═CH₂, —C(CH₃)═CH₂, —CH═CH—CH₃, —C≡C—CH₃, —CH₂—C—CH, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C═CH₃, —C≡C—CH═CH₂, —CH═CH—C═CH, —C═C≡C≡CH, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C≡CH, —C═C—C₃H₇, —C₂H₄—C≡C—CH₃, —CH₂—C≡C—C₂H₅, —CH₂—C═C—CH═CH₂, —CH₂—CH═CH—C≡CH, —CH₂—C≡C—C≡CH, —C═C—CH═CH—CH₃, —CH═CH—C≡C—CH₃, —C═C—C≡C—CH₃, —C≡C—CH₂—CH═CH₂, —CH═CH—CH₂—C≡CH, —C≡C—CH₂—C—CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅; and R₃ and R₃′ represent independently of each other

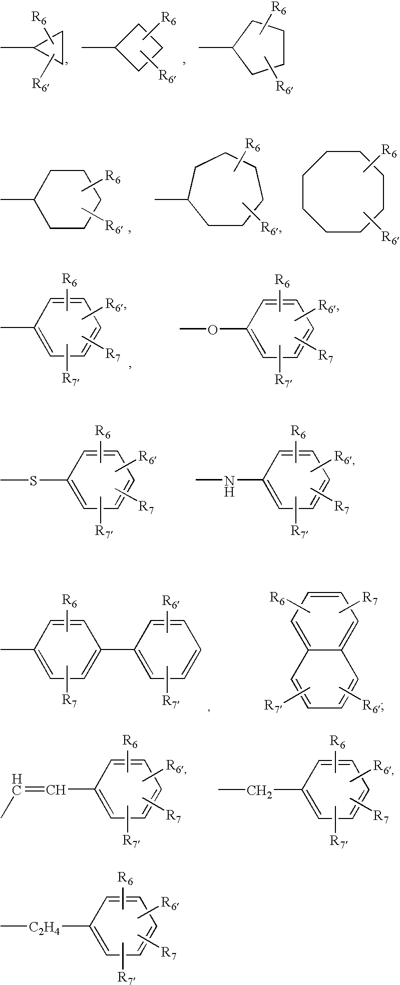

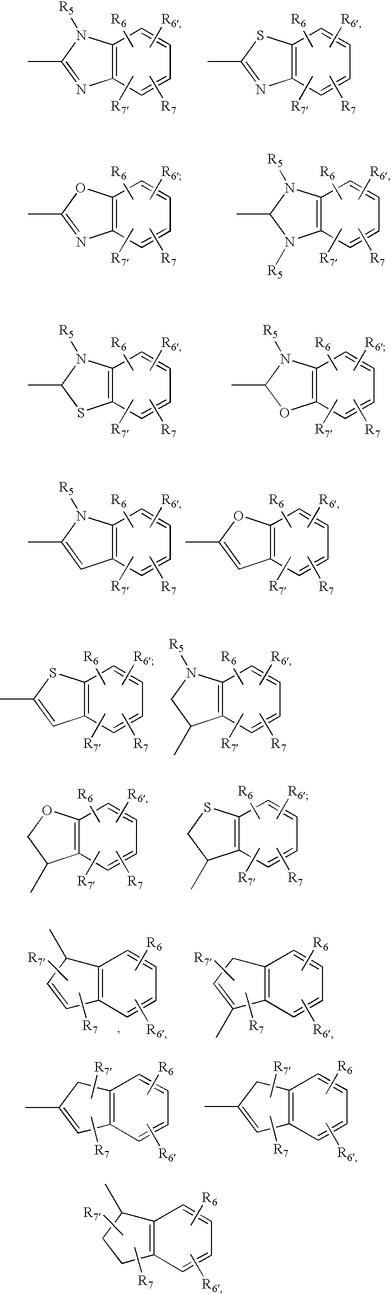

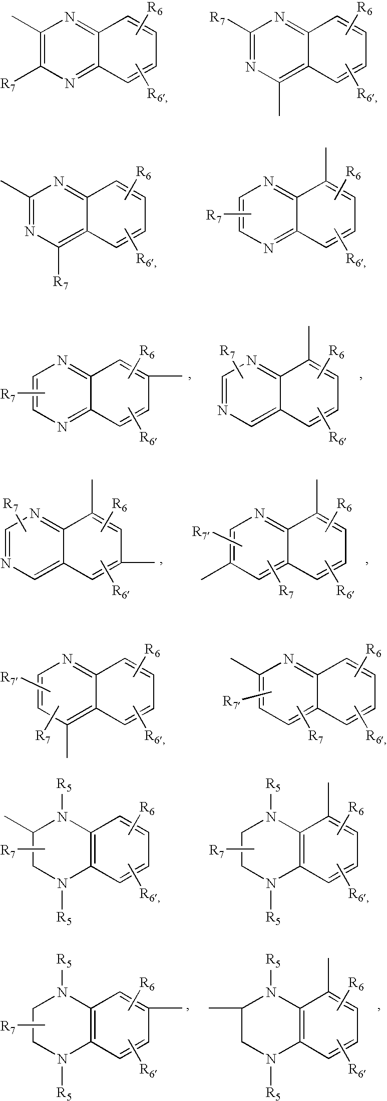

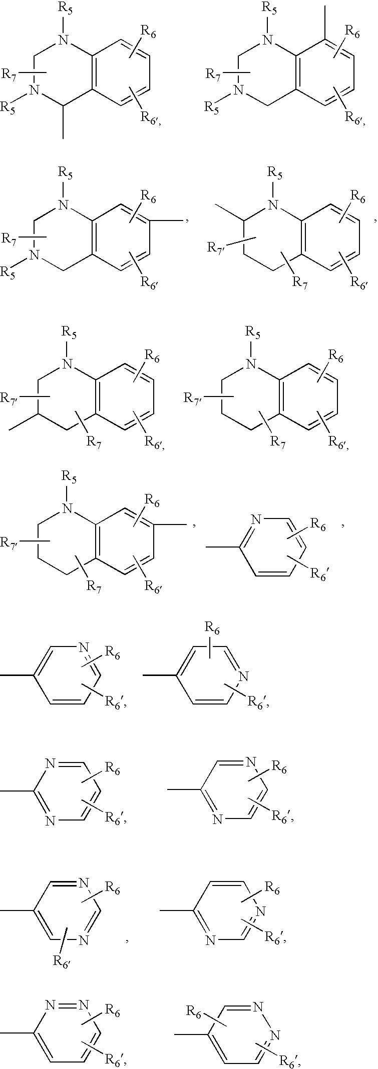

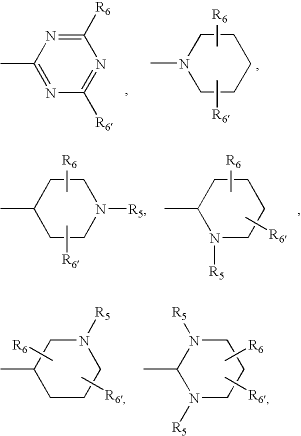

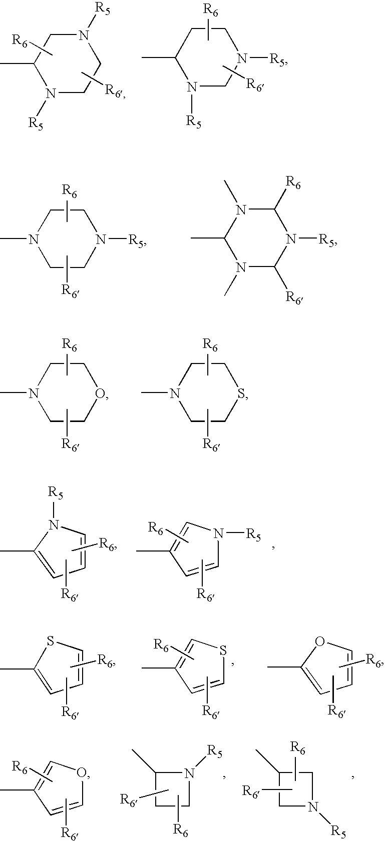

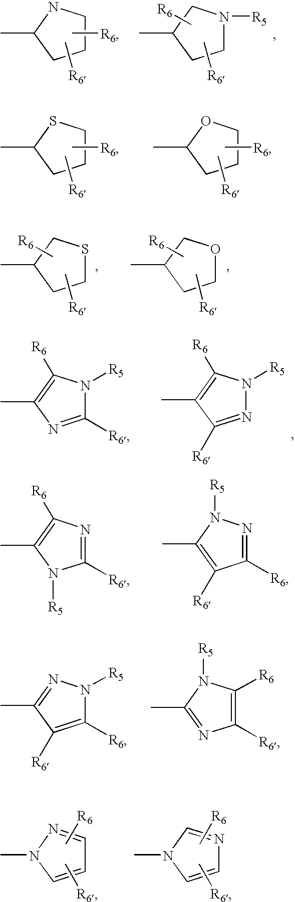

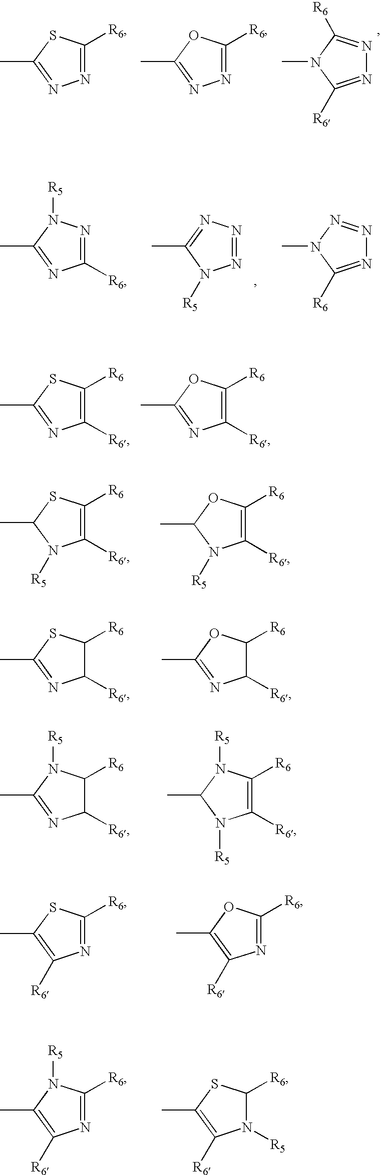

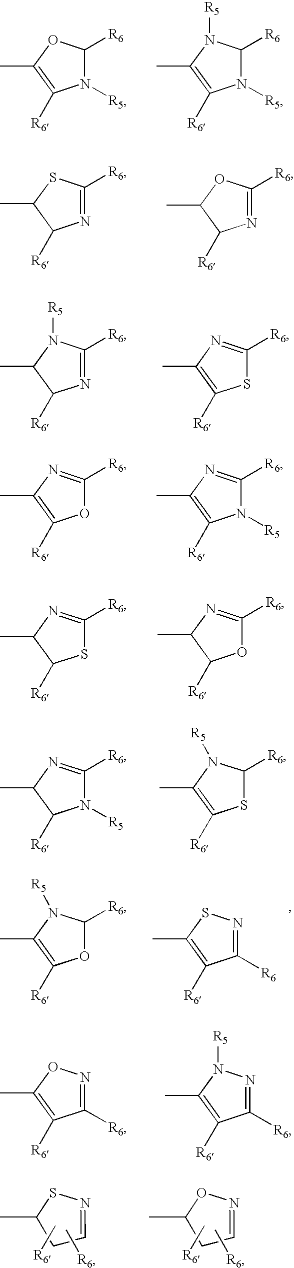

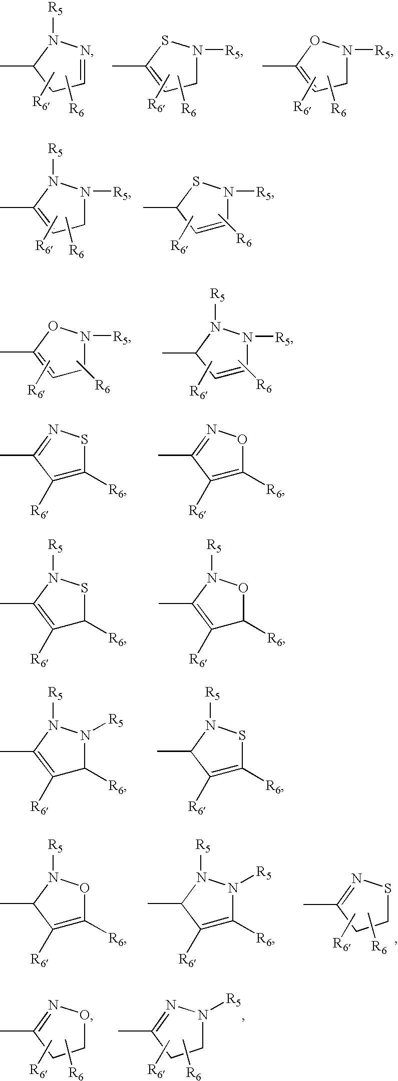

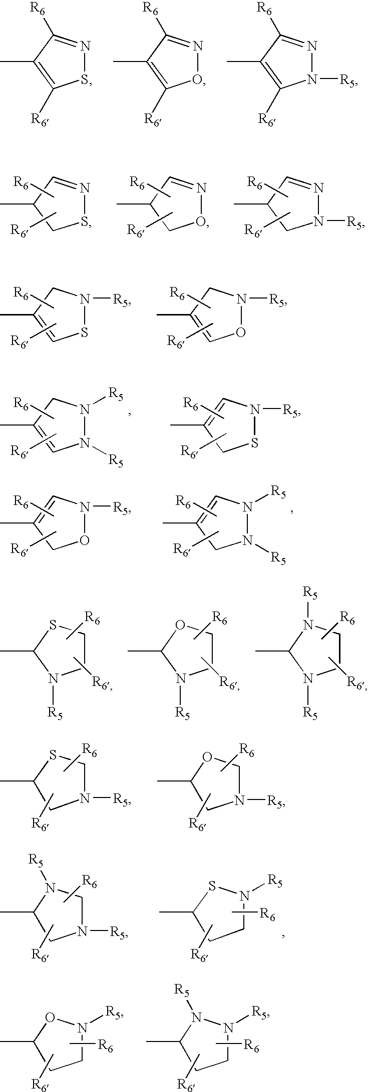

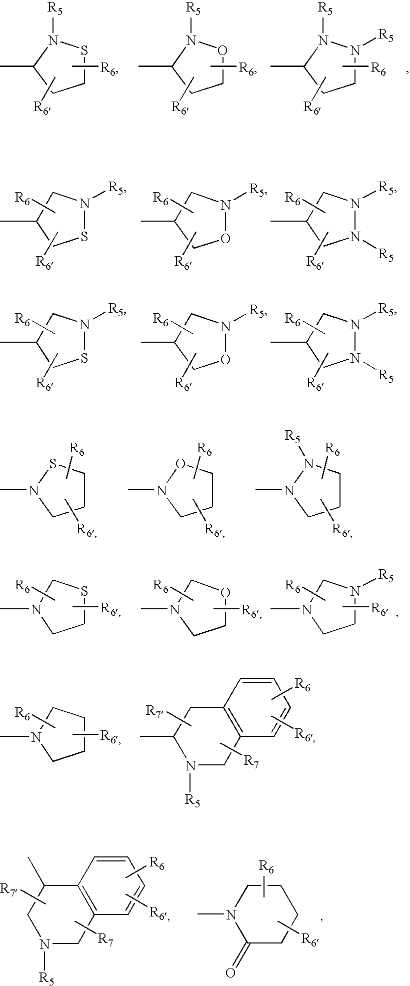

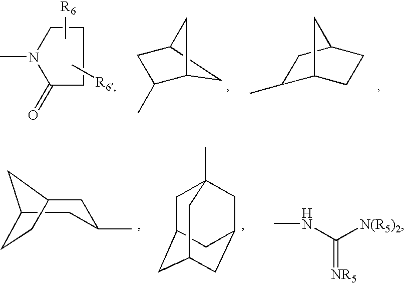

and R₅, R₆, R₆′, R₇, R₇′, L, X, Y, Z, n, m, o, p, q, r and s have the meanings as defined before.

In yet another preferred embodiment of the compounds according to general formula (I), R₁ represents —H or linear or branched C₁-C₆ alkyl, linear or branched C₂-C₆ alkenyl or linear or C₂-C₆ branched alkinyl, R₂ and R₄ represent independently of each other —H or linear or branched C₁-C₆ alkyl, linear or branched C₂-C₆ alkenyl, linear or branched C₂-C₆ alkinyl, —NH₂, —NO₂, —CN, R₃ or R₅′; R₃, R₃′, R₅, R₆, R₆′, R₇, R₇′, L, X, Y, Z, n, m, o, p, q, r and s have the meanings as defined before.

In a further preferred embodiment of the compounds according to general formula (I), R₁ represents —H or linear or branched C₁-C₆ alkyl, R₂ and R₄ represent independently of each other —H, —NH₂, linear or branched C₁-C₆ alkyl, R₃ and R₃′ are independently selected from the group comprising of: Halogen, X-aryl, which is optionally substituted by at least one of the groups R₆, R₆′, R₇ or R₇′, aryl, which is optionally substituted by at least one of the groups R₆, R₆′, R₇ or R₇′, partially or fully saturated 5 or 6 membered heterocyclic ring, which is optionally substituted by at least one of the groups R₆, R₆′, R₇ or R₇′; this heterocyclic ring can be fused to another 5 or 6 membered heterocyclic ring, which is optionally substituted by at least one of the groups R₆, R₆′, R₇ or R₇′, 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic ring, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′, guanidinyl group, optionally substituted by at least one R₅ group or a —Y—(CH₂)ₚ-Z group, wherein X, Y, Z and p have the meanings as defined above and R₅, R₆, R₆′, R₇ or R₇′, L, n, m, o, p, q, r and s have the meanings as defined above.

In yet another preferred embodiment of the compounds according to general formula (I), R₁ represents —H or linear or branched C₁-C₆ alkyl, R₂ and R₄ represent independently of each other —H, NH₂ or linear or branched C₁-C₆ alkyl, R₃ and R₃′ are independently selected from the group comprising of: Halogen, X-aryl, which is optionally substituted by at least one of the groups R₆, R₆′, R₇ or R₇′, aryl, which is optionally substituted by at least one of the groups R₆, R₆′, R₇ or R₇′, partially or fully saturated 5 or 6 membered heterocyclic ring, which is optionally substituted by at least one of the groups R₆, R₆′, R₇ or R₇′; this heterocyclic ring can be fused to another 5 or 6 membered heterocyclic ring, which is optionally substituted by at least one of the groups R₆, R₆′, R₇ or R₇′, 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic ring, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′, guanidinyl group, optionally substituted by at least one R₅ group or a —Y—(CH₂)ₚ-Z group, wherein X, Y, Z and p have the meanings as defined above, L represents —NR₅—SO₂—, —NR₅—CO—(CH₂)ₙ—, —NH—CO—NH—, —CO—NR₅— or —SO₂—NR₅—, R₅, R₆, R₆′, R₇ or R₇′, n, m, o, p, q, r, and s have the meanings as defined above.

In yet another preferred embodiment of the compounds according to general formula (I), R₁ represents —H or linear or branched C₁-C₆ alkyl, R₂ and R₄ represent independently of each other —H, or NH₂, R₃ and R₃′ are independently selected from the group comprising of: Halogen, X-aryl, which is optionally substituted by at least one of the groups R₆, R₆′, R₇ or R₇′, aryl, which is optionally substituted by at least one of the groups R₆, R₆′, R₇ or R₇′, partially or fully saturated 5 or 6 membered heterocyclic ring, which is optionally substituted by at least one of the groups R₆, R₆′, R₇ or R₇′; this ring can be fused to another 5 or 6 membered heterocyclic ring, which is optionally substituted by at least one of the groups R₆, R₆′, R₇ or R₇′, 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic ring, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′, guanidinyl group, optionally substituted by at least one R₅ group or a —Y—(CH₂)ₚ-Z group, wherein X, Y, Z and p have the meanings as defined above; L represents —NR₅—SO₂—, —NR₅—CO—(CH₂)s-, —NH—CO—NH—, —CO—NR₅— or —S0₂—NR₅—,

R₅ is selected from the group comprising: linear or branched C₁-C₆ alkyl, C₃-C₈ cycloalkyl, which is optionally substituted by at least one of the groups R₆, R₆′, R₇ or R₇′; C₄-C₁₂ bicycloalkyl, which is optionally substituted by at least one of the groups R₆, R₆′, R₇ or R₇′, aryl, which is optionally substituted by at least one of the groups R₆, R₆′, R₇ or R₇′, —CH₂-aryl, which is optionally substituted by at least one of the groups R₆, R₆′, R₇ or R₇′, partially or fully saturated 5 or 6 membered heterocyclic ring, which is optionally substituted by at least one of the groups R₆, R₆′, R₇ or R₇′; this ring can be fused to another 5 or 6 membered heterocyclic ring, which is optionally substituted by at least one of the groups R₆, R₆′, R₇ or R₇′, 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic ring, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′, —(CH₂)_{q}—COOR₁, wherein R₁ represents —H or a linear or branched C₁-C₆ alkyl, —(CH₂)ᵣ—N(R₁)₂, wherein R₁ represents independently —H or a linear or branched C₁-C₆ alkyl or —C(R₁)₃, wherein R₁ represents independently —H, a linear or branched C₁-C₆ alkyl or an aryl group, which is optionally substituted by R₆, R₆′, R₇ and R₇′; R₆, R₆′, R₇ or R₇′, n, m, o, p, q, r and s have the meanings as defined above.

In yet another preferred embodiment of the compounds according to general formula (I), R₁ represents —H or linear or branched C₁-C₆ alkyl, R₂ and R₄ represent independently of each other —H, or NH₂, R₃ and R₃′ are independently selected from the group comprising of: Halogen, X-aryl, which is optionally substituted by at least one of the groups R₆, R₆′, R₇ or R₇′, aryl, which is optionally substituted by at least one of the groups R₆, R₆′, R₇ or R₇′, partially or fully saturated 5 or 6 membered heterocyclic ring, which is optionally substituted by at least one of the groups R₆, R₆′, R₇ or R₇′; this ring can be fused to another 5 or 6 membered heterocyclic ring, which is optionally substituted by at least one of the groups R₆, R₆′, R₇ or R₇′, 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic ring, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′ or a —Y—(CH₂)ₚ-Z group, wherein X, Y, Z and p have the meanings as defined above;

L represents —NR₅—SO₂—, —NR₅—CO—(CH₂)n—, —NH—CO—NH—, —CO—NR₅— or —S0₂—NR₅—,

R₅ is selected from the group comprising:

linear or branched C₁-C₆ alkyl, aryl, which is optionally substituted by at least one of the groups R₆, R₆′, R₇ or R₇′; partially or fully saturated 5 or 6 membered heterocyclic ring, which is optionally substituted by at least one of the groups R₆, R₆′, R₇ or R₇′; this ring can be fused to another 5 or 6 membered heterocyclic ring, which is optionally substituted by at least one of the groups R₆, R₆′, R₇ or R₇′, 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic ring, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′, C₃-C₈ cycloalkyl, which is optionally substituted by at least one of the groups R₆, R₆′, R₇ or R₇′ or C₄-C₁₂ bicycloalkyl, which is optionally substituted by at least one of the groups R₆, R₆′, R₇ or R₇′;
R₆, R₆′, R₇ or R₇′ represent independently of each other: —H, —F, —Cl, —Br, —I, R₁, —OR₁, —N(R₁)₂, —CH═CH—COOR₁, —(CH₂)_{q}COOR₁, or a —O—(CH₂)ₜ-aryl, which is optionally substituted by at least one of the groups R₆, R₆′, R₇ or R₇′, wherein in these cases, R₁ is independently selected from —H or linear or branched C₁-C₆ alkyl and t is selected to be an integer from 0 to 6, n, m, o, p, q, r, and s have the meanings as defined above.

In yet another preferred embodiment of the compounds according to general formula (I) R₆, R₆′, R₇ or R₇′ represent independently of each other —H, linear or branched C₁-C₆ alkyl, —OR₁, —O—(CH₂)ₛ-aryl group, which is optionally substituted by at least one of the groups R₆, R₆′, R₇ or R₇′; —N(R₁)₂, —CH═CH—COOR₁—(CH₂)qCOOR₁, wherein in these cases, R₁ represents independently —H or linear or branched C₁-C₆ alkyl.

In a further embodiment of the compounds according to general formula (I), R₁ represents —H or linear or branched C₁-C₆ alkyl, R₂ and R₄ are independently selected from —H or —NH₂.

In yet another embodiment of the compounds as defined by general formula (I), each R₁ independently represents —H, linear or branched C₁-C₆ alkyl, linear or branched C₂-C₆ alkenyl or linear or branched C₂-C₆ alkinyl or benzyl, preferably —H, or linear or branched C₁-C₆ alkyl,

R₂ and R₄ are independently selected from the group consisting of: —H, —CN, —NH₂, —NO₂, linear or branched C₁-C₆ alkyl, linear or branched C₂-C₆ alkenyl or C₂-C₆ linear or branched alkinyl, and preferably are independently selected from —H or —NH₂,

R₃ is selected from the group consisting of halogen, pyridinyl, thienyl, phenyl and biphenyl, preferably phenyl, which are optionally substituted by at least one of the groups R₆, R₆′, R₇ or R₇′, R₆, wherein R₆, R₆′, R₇ or R₇′ are preferably selected from halogen, such as —F, —Cl, —Br, or I, —OR₁, —N(R₁)₂, wherein in these groups each R₁ is preferably independently selected from —H or linear or branched C₁-C₆ alkyl or benzyl, n is selected to be 0, m is 0 or 1, preferably 1, o is 0 or 1, preferably 1,

R₃′ is phenyl, optionally substituted by at least one of the groups R₆, R₆′, R₇ or R₇′, wherein R₆, R₆′, R₇ or R₇′ are preferably selected from halogen, such as —F, —Cl, —Br, or I, —OR₁, —N(R₁)₂, wherein in these groups each R₁ is preferably independently selected from —H or linear or branched C₁-C₆ alkyl,

L is —NH—CO—(CH₂)s—, wherein s is preferably 0 or 1, or —NH—SO₂—, and preferably is —NH—CO—, and

R₅ is selected from the group consisting of: a partially or fully saturated 5 or 6 membered heterocyclic ring, which is optionally substituted by at least one of the groups R₆, R₆′, R₇ or R₇′; this heterocyclic ring can be fused to another 5 or 6 membered heterocyclic ring, which is optionally substituted by at least one of the groups R₆, R₆′, R₇ or R₇′, wherein R₆, R₆′, R₇ or R₇′ in these heterocyclic rings are preferably selected from halogen, such as —F, —Cl, —Br, or I, —OR₁, —N(R₁)₂, wherein in these groups each R₁ is preferably independently selected from —H or linear or branched C₁-C₆ alkyl, and wherein R₅ is preferably selected from the group consisting of azetidinyl, pyrrolidinyl, or piperidinyl, each of these heterocycles optionally substituted in the above indicated manner, a 5 or 6 membered heteroaryl ring, which is optionally substituted by at least one of the groups R₆, R₆′, R₇ or R₇′; this heteroaryl ring can be fused to another partially or fully saturated 5 or 6 membered heterocyclic group, which is optionally substituted by at least one of the groups R₆, R₆′, R₇ or R₇′ or to a 5 or 6 membered heteroaryl ring, which is optionally substituted by at least one of the groups R₆, R₆′, R₇ or R₇′; wherein R₆, R₆′, R₇ or R₇′ in these rings are preferably selected from halogen, such as —F, —Cl, —Br, or I, —OR₁, —N(R₁)₂, wherein in these groups each R₁ is preferably independently selected from —H or linear or branched C₁-C₆ alkyl, and wherein R₅ is preferably selected from the group consisting of benzoxazoly, benzimidazolyl, chinolinyl, imidazol, benzothiazolyl, 1, 2, 3, 4,-Tetrahydro-isoquinolinyl, or pyridinyl, each of these groups optionally being substituted in the above indicated manner,

phenyl, optionally substituted by at least one of the groups R₆, R₆′, R₇ or R₇′, wherein R₆, R₆′, R₇ or R₇′ are preferably selected from halogen, such as —F, —Cl, —Br, or I, —OR₁, —N(R₁)₂, wherein in these groups each R₁ is preferably independently selected from —H or linear or branched C₁-C₆ alkyl, or C₁-C₆-alkyl or a C₃-C₈-cycloalkyl, which is optionally substituted by at least one of the groups R₆, R₆′, R₇ or R₇′, wherein R₆, R₆′, R₇ or R₇′ are preferably selected from halogen, such as —F, —Cl, —Br, or I, —OR₁, —N(R₁)₂, wherein in these groups each R₁ is preferably independently selected from —H or linear or branched C₁-C₆ alkyl.

In a further embodiment, the compound of the present invention defined by general formula (I) represents a chiral compound. The compound can be a racemate or a R or a S enantiomer.

As used herein the terms linear or branched C₁-C₈ alkyl, linear or branched C₂-C₆ alkenyl or linear or branched C₂-C₆ alkinyl are meant to include the following alkyls, alkenyls or alkinyls:

—CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂—C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂Hs, —C(CH₃)₂—C₃H₇, —C(CH₃)₂—CH(CH₃)₂, —C₂H₄—C(CH₃)₃, —CH(CH₃)—C(CH₃)₃, —(CH₂)₆—CH₃, —CH(CH₃)—(CH₂)₄—CH₃—(CH₂)₂—CH(CH₃)—(CH₂)₂—CH₃, —(CH₂)₃—CH(CH₃)—C₂H₅, —(CH₂)₄—CH(CH₃)₂, —C(CH₃)₂—(CH₂)₃—CH₃, —CH₂—C(CH₃)₂—(CH₂)₂—CH₃, —(CH₂)₂—C(CH₃)₂—C₂H₅, —(CH₂)₄—CH(CH₃)₂, —(CH₂)₃—C(CH₃)₃, —CH(C₂H₅)—(CH₂)₃—CH₃, —(CH₂)₃—CH(C₂Hs)-CH₃, —C(C₂H₅)₃, —CH₂—C(C₂H₅)₂—CH₃, —(CH₂)₂—CH(C₂Hs)₂, —CH(C₃H₇)—(CH₂)₂—CH₃, —CH₂—CH(C₃H₇)—C₂H₅, —(CH₂)₂—CH(C₃H₇)—CH₃, —(CH₂)₇—CH₃, —CH(CH₃)—(CH₂)₅—CH₃, —(CH₂)₂—CH(CH₃)—(CH₂)₃—CH₃, —(CH₂)₃—CH(CH₃)—(CH₂)₂—CH₃, —(CH₂)₄—CH(CH₃)—C₂H₅, —(CH₂)₅—CH(CH₃)₂, —(CH₂)₄—C(CH₃)₃, —CH(C₂H₅)—(CH₂)₄—CH₃, —(CH₂)₂—CH(C₂H₅)—(CH₂)₂—CH₃, —(CH₂)₃—CH(C₂H₅)₂, —CH(C₃H₇)—(CH₂)₃—CH₃, —CH₂—CH(C₃H₇)—(CH₂)₂—CH₃, —(CH₂)₂—CH(C₃H₇)—C₂H₅, —(CH₂)₃—CH(C₃H₇)—CH₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₄H₉)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C (CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, —CH═CH—CH═CH—CH═CH₂, —C═CH, —C≡C—CH₃, —CH₂—C═CH, —C₂H₄—C≡CH, —CH₂—C≡C—CH₃, —C≡C—C₂H₅, —C₃H₆—C═CH, —C₂H₄—C≡C—CH₃, —CH₂—C≡C—C₂H₅, —C═C—C₃H₇, —CH(CH₃)—C≡CH, —CH₂—CH(CH₃)—C═CH, —CH(CH₃)—CH₂—C═CH, —CH(CH₃)—C═C—CH₃, —C₄H₈—C—CH, —C₃H₆—C≡C—CH₃, —C₂H₄—C≡C—C₂H₅, —CH₂—C≡C—C₃H₇, —C≡C—C₄H₉, —C₂H₄—CH(CH₃)—C═CH, —CH₂—CH(CH₃)—CH₂—C≡CH, —CH(CH₃)—C₂H₄—C≡CH, —CH₂—CH(CH₃)—C═C—CH₃, —CH(CH₃)—CH₂—C≡C—CH₃, —CH(CH₃)—C═C—C₂H₅, —CH₂—C≡C—CH(CH₃)₂, —C═C—CH(CH₃)—C₂H₅, —C═C—CH₂—CH(CH₃)₂, —C═C—C(CH₃)₃, —CH(C₂H₅)—C═C—CH₃, —C(CH₃)₂—C═C—CH₃, —CH(C₂H₅)—CH₂—C═CH, —CH₂—CH(C₂H₅)—C≡CH, —C(CH₃)₂—CH₂—C═CH, —CH₂—C(CH₃)₂—C═CH, —CH(CH₃)—CH(CH₃)—C≡CH, —CH(C₃H₇)—C═CH, —C(CH₃)(C₂H₅)—C≡CH, —C═C—C═CH, —CH₂—C≡C—C═CH, —C═C—C═C—CH₃, —CH(C═CH)₂, —C₂H₄—C═C—C═CH, —CH₂—C═C—CH₂—C═CH, —C≡C—C₂H₄—C≡CH, —CH₂—C≡C—C≡C—CH₃, —C≡C—CH₂—C≡C—CH₃, —C═C—C═C—C₂Hs, —C═C—CH(CHs)-C═CH, —CH(CH₃)—C═C—C═CH, —CH(C—CH)—CH₂—C≡CH, —C(C≡CH)₂—CH₃, —CH₂—CH(C—CH)₂, —CH(C—CH)—C═C—CH₃, —C≡C—CH═CH₂, —CH═CH—C—CH, —CH₂—C═C—CH═CH₂, —CH₂—CH═CH—C═CH, —C═C—CH═CH—CH₃, —CH═CH—C═C—CH₃, —C═C—CH₂—CH═CH₂, —CH═CH—CH₂—C—CH, —C═C—CH₂—C═CH, —C(CH₃)═CH—C═CH, —CH═C(CH₃)—C—CH, —C≡C—C(CH₃)═CH₂, and —C≡C—C≡C—C≡CH.

The term linear or branched C₁-C₆ substituted or unsubstituted alkyl, linear or branched C₁-C₄ substituted or unsubstituted alkyl or linear or branched C₂-C₄ alkenyl is meant to include the respective subgroup out of the above groups.

The term C₃-C₈ cycloalkyl denotes the following cycloalkyls:

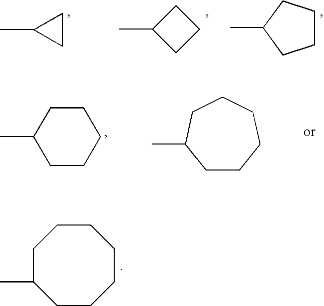

The term C₅-C₁₂ bicycloalkyl is meant to include the following bicycloalkyls:

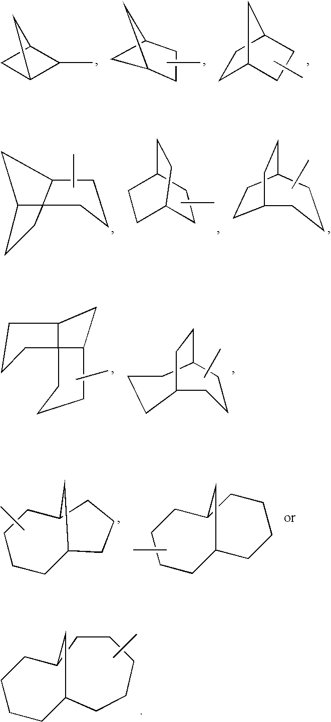

The term aryl denotes an aromatic mono- or bicyclic 6 to 10 membered ring system such as phenyl, naphthyl, 3-chlorophenyl, 2,6-dibromophenyl, 2,4,6 tribromophenyl, 4,7-dichloronaphthyl, and preferably phenyl or naphthyl.

The term heterocyclyl is meant to include a 5 to 10 membered mono- or bicyclic ringsystem, containing one to three heteroatoms independently selected from oxygen, sulfur or nitrogen and is preferably selected from the group comprising: Aziridinyl, azetidinyl, pyrrolidinyl, tetrahydrofuranyl, tetrahydrothiophenyl, piperidinyl, piperadizinyl, piperazinyl, tetrahydropyranyl, tetrahydrothiopyranyl or morpholinyl.

The term heterocyclyl further comprises all heteroaryls as defined below, wherein all double bonds of the correspondent heteroaryls are replaced by single bonds.

The term heteroaryl denotes a partially or fully unsaturated 5 to 10 membered mono- or bicyclic ringsystem, containing one to three heteroatoms independently selected from oxygen, sulfur or nitrogen and is preferably selected from the group consisting of:

Pyrrolyl, furanyl, thiophenyl, thienyl, imidazolyl, pyrazolyl, thiazolyl, oxazolyl, isothiazolyl, isoxazolyl, pyridinyl, pyridyl, pyrimidinyl, pyrimidyl, pyrazinyl, pyrazyl, pyradizinyl, pyradizyl, 3-methylpyridyl, benzothienyl, 4-ethylbenzothienyl, 3,4-diethylfuranyl, pyrrolyl, tetrahydroquinolyl, quinolyl, tetrahydroisoquinolinyl, isoquinolinyl, benzoimidazolyl, benzothiazolyl, benzooxyzolyl, benzo [1,3] dioxolyl, indolyl, benzofuranyl, benzothiophenyl, indazolyl or chrom-2-onyl.

It is to be understood, that the term heteroaryl also comprises partially unsaturated 5 to 10 membered mono- or bicyclic ringsystem, wherein one up to 4 double bonds of the ringsystem are replaced by a single bond and wherein the ringsystem contains at least one double bond.

In a preferred embodiment, R¹ in the compounds according to the general formula (I) is selected from —H or linear or branched substituted or unsubstituted C₁-C₆ alkyl, preferably from —H or linear or branched substituted or unsubstituted C₁-C₄ alkyl, more preferably from —H or —CH₃, and is most preferably —H.

In a further preferred embodiment, R² in the compounds according to the general formula (I) is selected from —H, —NH₂ or linear or branched substituted or unsubstituted C₁-C₆ alkyl, preferably from —H or linear or branched substituted or unsubstituted C₁-C₄ alkyl, and is more preferably —H.

In yet another preferred embodiment, R⁴ in the compounds according to the general formula (I) is selected from —H, —NH₂ or linear or branched substituted or unsubstituted C₁-C₆ alkyl, preferably from —H or linear or branched substituted or unsubstituted C₁-C₄ alkyl, more preferably from —H or —CH₃, and is most preferably —H.

In yet another preferred embodiment, m in the compounds according to the general formula (I) is selected to be 0, R³ is selected from the group comprising:

Substituted or unsubstituted aryl, preferably substituted or unsubstituted phenyl, more preferably substituted phenyl, and R⁵ is selected from the group consisting of: Substituted or unsubstituted aryl, preferably substituted or unsubstituted phenyl, more preferably substituted phenyl, or —(CH₂)ₒ—Y, wherein o is an integer from 0 to 4 and Y represents substituted or unsubstituted heteroaryl, preferably unsubstituted heteroaryl.

In yet another preferred embodiment, R³ and R⁵ in the compounds according to the general formula (I) represent phenyl, wherein each phenyl is independently of each other partially or fully substituted with members selected from the group consisting of:

Linear or branched substituted or unsubstituted C₁-C₆ alkyl, preferably linear or branched substituted or unsubstituted C₁-C₄ alkyl, more preferably —CH₃, linear or branched C₁-C₆ alkoxy, preferably linear or branched C₁-C₄ alkoxy, more preferably —OCH₃, —O—(CH₂)ᵤ-Phenyl, wherein u is an integer from 0 to 6, preferably from 0 to 4, more preferably from 0 to 2,

—NR²⁰R²¹, wherein R²⁰ and R²¹ are independently of each other selected from —H or linear or branched substituted or unsubstituted C₁-C₆ alkyl, more preferably from —H or linear or branched substituted or unsubstituted C₁-C₄ alkyl, and are most preferably —H, —COOR²², wherein R²² represents linear or branched substituted or unsubstituted C₁-C₆ alkyl, preferably linear or branched substituted or unsubstituted C₁-C₄ alkyl, more preferably —CH₃, or phenyl is substituted with heteroaryl selected from benzoimidazolyl, benzothiazolyl or benzoxazolyl, and wherein each phenyl is preferably mono-, di- or trisubstituted, more preferably mono- or disubstituted.

In yet another preferred embodiment, R⁵ in the compounds according to the general formula (I) represents —(CH₂)ₒ—Y, wherein o is selected to be 2 and wherein Y represents unsubstituted pyridinyl, preferably unsubstituted 4-pyridinyl.

In another preferred embodiment, m in the compounds according to the general formula (I) is selected to be 1.

In yet another preferred embodiment, R³ in the compounds according to the general formula (I) is selected from the group comprising: —Cl, —Br, —I, preferably —Cl or —Br, more preferably —Cl, substituted or unsubstituted aryl, substituted or unsubstituted —CH═CH-aryl, preferably substituted or unsubstituted —CH═CH-phenyl, more preferably unsubstituted —CH═CH-phenyl, substituted or unsubstituted heteroaryl, substituted or unsubstituted heterocyclyl, preferably substituted heterocyclyl, substituted or unsubstituted C₃-C₈ cycloalkyl, substituted or unsubstituted W-heterocyclyl, wherein W is selected to be —NH, preferably substituted —NH-heterocyclyl or R³ represents —NH—(CH₂)ₙ—X, wherein n is an integer from 0 to 4, preferably from 0 to 2, and X is selected from —OH, —NH₂ or substituted or unsubstituted C₃-C₈ cycloalkyl, preferably unsubstituted cycloalkyl, more preferably unsubstituted cyclohexyl. In yet another preferred embodiment, R³ in the compounds according to the general formula (I) represents partially or fully substituted heterocyclyl, wherein the heterocyclyl is selected from the group consisting of: Pyrrolidinyl, piperidinyl, piperazinyl, morpholinyl, preferably substituted piperazinyl, wherein piperazinyl is N-substituted with linear or branched substituted or unsubstituted C₁-C₄ alkyl, preferably —CH₃.

In yet another preferred embodiment, R³ in the compounds according to general formula (I) represents substituted or unsubstituted heteroaryl, wherein the heteroaryl is selected from the group comprising: Pyridinyl, pyridyl, pyridazinyl, pyrimidinyl, imidazolyl, pyrimidyl, pyrazinyl, pyrazyl, thiophenyl, thienyl, furanyl or pyrrolyl, thiazolyl, oxazolyl, isothiazolyl, isoxazolyl, pyrazyl, pyradizinyl, pyradizyl, 3-methylpyridyl, benzothienyl, 4-ethylbenzothienyl, 3,4-diethylfuranyl, pyrrolyl, tetrahydroquinolyl, quinolyl, tetrahydroisoquinolinyl, isoquinolinyl, benzoimidazolyl, benzothiazolyl, benzooxyzolyl, benzo [1,3] dioxolyl, indolyl, benzofuranyl, benzothiophenyl, indazolyl or chrom-2-onyl and preferably pyridinyl, pyridyl, pyrimidinyl, pyrimidyl, thiophenyl or furanyl, more preferably 2-pyridinyl, 3-pyridinyl, 4-pyridinyl, pyrimidinyl, 2-thiophenyl or 2-furanyl, and wherein the substituted heteroaryl is selected from furanyl, thiophenyl or pyridinyl, preferably 3-pyridinyl or 2-thiophenyl, partially or fully substituted with linear or branched C₁-C₄ alkoxy, preferably with —OCH₃, or with —CO—CH₃, and wherein the pyridinyl or thiophenyl are preferably monosubstituted.

In another preferred embodiment, R³ in the compounds according to general formula (I) represents substituted or unsubstituted phenyl, preferably substituted phenyl, wherein within this embodiment phenyl is partially or fully substituted with members of the group consisting of: —F, —Cl, —Br, —I, preferably —F or —Cl, —CN, —NO₂,

linear or branched substituted or unsubstituted C₁-C₆ alkyl, preferably linear or branched substituted or unsubstituted C₁-C₄ alkyl, linear or branched C₂-C₆ alkenyl, preferably linear or branched C₂-C₄ alkenyl, substituted or unsubstituted phenyl, preferably unsubstituted phenyl,
linear or branched C₁-C₆ alkoxy, preferably linear or branched C₁-C₄ alkoxy, —O— (CH₂)ᵥ—R, wherein v is an integer from 0 to 6, preferably from 0 to 4 and R is selected from the group consisting of:

Phenyl, —O-phenyl, linear or branched substituted or unsubstituted C₁-C₄ haloalkyl, heterocyclyl, or —NR²³R²⁴, wherein R²³ and R²⁴ are independently of each other selected from —H or linear or branched substituted or unsubstituted C₁-C₆ alkyl, preferably from —H or linear or branched substituted or unsubstituted C₁-C₄ alkyl, linear or branched C₁-C₆ haloalkyl, preferably linear or branched C₁-C₄ haloalkyl, linear or branched C₁-C₆ thioalkyl, preferably linear or branched C₁-C₄ thioalkyl,

—(CH₂)_{w}-Q, wherein w is selected to be an integer from 0 to 6, preferably from 0 to 4 and Q is selected from heterocyclyl, —OH, —NR²⁵R²⁶, wherein R²⁵ and R²⁶ are independently of each other selected from —H, linear or branched substituted or unsubstituted C₁-C₆ alkyl, preferably —H or linear or branched substituted or unsubstituted C₁-C₄ alkyl, or (CH₂)_{y}—O—CH₃, wherein y is selected to be an integer from 0 to 6, preferably from 0 to 4, or Q represents linear or branched C₁-C₆ alkoxy, preferably linear or branched C₁-C₄ alkoxy, —(CH₂)_{y}—COR²⁷, wherein y is an integer from 0 to 6, preferably from 0 to 4, and R²⁷ is selected from the group comprising: —H, linear or branched substituted or unsubstituted C₁-C₆ alkyl, preferably linear or branched substituted or unsubstituted C₁-C₄ alkyl, —OR²⁸, wherein R²⁸ is selected from —H or linear or branched substituted or unsubstituted C₁-C₆ alkyl, preferably linear or branched substituted or unsubstituted C₁-C₄ alkyl, or R²⁸ is selected from —NR²R¹, wherein R²⁹ and R¹⁰ are independently of each other selected from —H, linear or branched substituted or unsubstituted C₁-C₆ alkyl or C₃-C₈ cycloalkyl, preferably from —H, linear or branched substituted or unsubstituted C₁-C₄ alkyl or C₄-C₆ cycloalkyl, preferably from —H, linear or branched substituted or unsubstituted C₁-C₄ alkyl or C₄-C₆ cycloalkyl, —CH═CH—COOH, —CH═CH—COOCH₃ or —NH-T-R³¹, wherein T is selected from —CO— or —SO₂— and R³¹ represents linear or branched substituted or unsubstituted C₁-C₆ alkyl, preferably linear or branched C₁-C₄ alkyl, and wherein phenyl is mono-, di- or trisubstituted, preferably mono- or disubstituted, and wherein within this embodiment, it is especially preferred, that phenyl is substituted with members of the group consisting of:

—F, —Cl, —CN, —C₂H₅, —CH(CH₃)₂, —CH═CH₂, —OCH₃, —OC₂H₅, —OCH(CH₃)₂, —O-Phenyl, —O—CH₂-Phenyl, —O—(CH₂)₂—O-Phenyl, -0-(CH₂)₂—N(CH₃)₂, —O—(CH₂)₃—N(CH₃)₂, —O—(CH₂)₃—NH₂, —OCF₃, —OH, —CH₂—OH, —CH₂—OCH₃, —SCH₃, —NH₂, —N(CH₃)₂, —CH₂—NH₂, —CH₂—N(CH₃)₂, —CH═CH—COOH, —CH═CH—COOCH₃, —COOH, —(CH₂)₂—COOH, —COOCH₃, —CF₃, Phenyl, —C(O)—H, —C(O)—CH₃, —C(O)—NH₂, —C(O)—NHCH(CH₃)₂, —NH—CO—CH₃—NH—SO₂—CH₃, —CH₂—N(CH₃)—(CH₂)₂—O—CH₃,

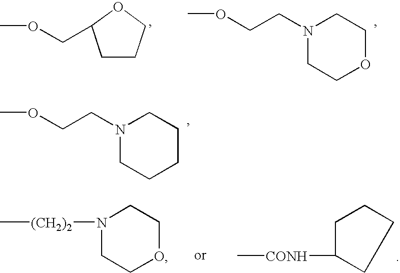

preferably phenyl is substituted with —OCH₃, —O—CH₂-Phenyl, —OH, —OCH(CH₃)₂ or NH₂.

In a further preferred embodiment, R⁵ in the compounds according to the general formula (I) is selected from substituted or unsubstituted aryl, substituted or unsubstituted heteroaryl or substituted or unsubstituted heterocyclyl.

In yet another preferred embodiment, R⁵ in the compounds according to the general formula (I) represents substituted or unsubstituted aryl, preferably substituted or unsubstituted phenyl, more preferably unsubstituted phenyl.

It is especially preferred, if R⁵ in the compounds according to the general formula (I) represents a substituted phenyl, that phenyl is partially or fully substituted with linear or branched substituted or unsubstituted C₁-C₆ alkyl, preferably with linear or branched substituted or unsubstituted C₁-C₄ alkyl, more preferably with —CH₃ or phenyl is partially or fully substituted with —O—(CH₂)ᵤ-Phenyl, wherein u is an integer from 0 to 6, preferably from 0 to 4, more preferably from 0 to 2, and is most preferably 1, and wherein phenyl is preferably mono substituted.

In a further preferred embodiment, L in the compounds according to the general formula (I) is selected from the group comprising: —NR¹⁴—SO₂—, wherein R¹⁴ is selected from —H, linear or branched substituted or unsubstituted C₁-C₄ alkyl, —SO₂—R¹⁵—, —R¹⁵—S0₂—, wherein R¹⁵ is selected from linear or branched substituted or unsubstituted C₁-C₄ alkylen, or R¹⁴ represents —(CH₂)ᵣCOOR¹⁶, wherein r is an integer from 0 to 4 and R¹⁶ is selected from —H or linear or branched substituted or unsubstituted C₁-C₄ alkyl, —NR¹⁷—CO—, wherein R¹⁷ is selected from —H, linear or branched substituted or unsubstituted C₁-C₄ alkyl, or a —(CH₂)ₛ-group, wherein s is an integer from 1 to 3, preferably s is selected to be 1, and wherein if R⁶ represents a —(CH₂)_{q}-group, wherein q is an integer from 1 to 3, preferably q is selected to be 2 and R¹⁷ represents a methylene chain —(CH₂)ₛ-group, R⁶ and R¹⁷ may form together a 5 to 8 membered ring system, preferably R⁶ and R¹⁷ form together a 5 membered ring system

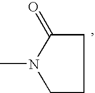

—SO₂—NR¹⁸—,

wherein R¹⁸ is selected from —H or linear or branched substituted or unsubstituted C₁-C₄ alkyl,

—CO—NR¹⁹—,

wherein R¹⁹ is selected from —H, linear or branched substituted or unsubstituted C₁-C₄ alkyl, or a —(CH₂)ₜ-A- group, wherein t is an integer from 1 to 3 and A is selected from N or O, and wherein if R⁶ represents a —(CH₂)_{q}-group wherein q is an integer from 1 to 3, preferably q is selected to be 2 and R¹⁹ represents a —(CH₂)ₜ-A- group, wherein t is selected to be 2 and A represents O, R⁶ and R¹⁹ may form together a 6-membered ring system

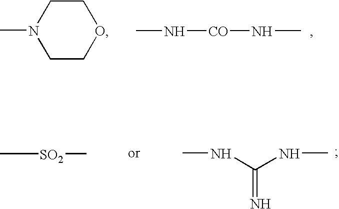

and wherein within this embodiment, it is especially preferred, that if R⁵ represents phenyl, L is preferably in meta- or para-position of the phenyl.

In yet another preferred embodiment, L in the compounds according to the general formula (I) is selected from the group consisting of:

—NR¹⁴—SO₂—,

wherein R¹⁴ is selected from —H, —(CH₂)₂—CH₃, —SO₂—R¹⁵ or —R¹⁵—SO₂—, wherein R¹⁵ represents linear or branched substituted or unsubstituted C₁-C₄ alkylen or —(CH₂)₂—CH₃, or —(CH₂)ᵣ—COOR⁶, wherein r is selected to be an integer from 0 to 2, and is preferably 1, and R¹⁶ represents —CH₃,

—NR¹⁷—CO—, —SO₂—NR¹⁸—, —CO—NR¹⁹—

wherein R¹⁷, R¹⁸ and R¹⁹ represent —H, —NH—CO—NH— or —SO₂—, wherein within this embodiment it is especially preferred, that L is selected from —NH—S0₂—, —NH—CO—, —CO—NH—, —S0₂—NH—, —NH—CO—NH— or —SO₂—.

In yet another preferred embodiment, R⁶ in the compounds according to the general formula (I) is selected from the group comprising: —H, linear or branched substituted or unsubstituted C₁-C₈ alkyl, substituted or unsubstituted aryl, substituted or unsubstituted heteroaryl, substituted or unsubstituted heterocyclyl, substituted pyrrolidinyl, substituted or unsubstituted C₃-C₈ cycloalkyl, disubstituted cyclohexyl, cyclopentyl, substituted or unsubstituted C₅-C₁₂ bicycloalkyl, substituted or unsubstituted adamantyl, or —(CH₂)ₚ-Z, wherein p is an integer from 0 to 4 and Z is selected from the group comprising:

substituted or unsubstituted aryl, preferably unsubstituted aryl, substituted or unsubstituted heteroaryl, substituted or unsubstituted heterocyclyl, —N(R⁷-R⁸), wherein R⁷ and R⁸ represent independently from each other —H, or linear or branched C₁-C₆ alkyl, or Z represents —(CR⁹R¹⁰R¹¹), wherein R⁹, R¹⁰ and R¹¹ are independently of each other selected from the group consisting of: —H, linear or branched substituted or unsubstituted C₁-C₄ alkyl, substituted or unsubstituted aryl or —N(R¹²R¹³), wherein R¹² and R¹³ represent independently of each other —H or linear or branched substituted or unsubstituted C₁-C₄ alkyl, and wherein if Z is selected from substituted or unsubstituted aryl, preferably unsubstituted aryl, substituted or unsubstituted heteroaryl, substituted or unsubstituted heterocyclyl, p can not be selected to be 0.

In yet another preferred embodiment, R⁶ in the compounds according to the general formula (I) is selected from the group consisting of:

—H, linear or branched substituted or unsubstituted C₁-C₆ alkyl, substituted or unsubstituted aryl, substituted or unsubstituted heteroaryl, substituted or unsubstituted heterocyclyl, substituted or unsubstituted C₃-C₈ cycloalkyl, unsubstituted C₅-C₁₂ bicycloalkyl, preferably unsubstituted bicyclo[2.2.1]heptanyl, unsubstituted adamantyl or —(CH₂)ₚ-Z, wherein p is an integer from 0 to 2 and Z is selected from the group comprising:

substituted or unsubstituted phenyl, substituted or unsubstituted heterocyclyl, —N(R⁷R⁸), wherein R⁷ and R⁸ represent, independently from each other —H, or linear or branched substituted or unsubstituted C₁-C₄ alkyl, or Z represents —(CR⁹R¹⁰R¹¹), wherein R⁹, R¹⁰ and R¹¹ are independently of each other selected from the group consisting of: —H, linear or branched substituted or unsubstituted C₁-C₆ alkyl, unsubstituted aryl or —N(R¹²R¹³), wherein R¹² and R¹³ represent independently of each other —H or linear or branched substituted or unsubstituted C₁-C₄ alkyl.

In yet another preferred embodiment, R⁶ in the compounds according to the general formula (I) represents —H or linear or branched C₁-C₆ alkyl, preferably —H, —CH₃, —C₂H₅, —C₃H₇, —CH(CH₃)₂, —C(CH₃)₃ or —CH₂—C(CH₃)₃, more preferably —H, —CH₃ or —C(CH₃)₃.

In yet another preferred embodiment, R⁶ in the compounds according to the general formula (I) represents substituted or unsubstituted aryl, such as substituted or unsubstituted phenyl or naphtyl, wherein if R⁶ represents substituted naphthyl, napthyl is partially or fully substituted with —OH or linear or branched C₁-C₄ alkoxy, preferably —OH and wherein napthyl is preferably monosubstituted,

or wherein if R⁶ represents substituted phenyl, phenyl is partially or fully substituted with members of the group comprising:

Phenyl, linear or branched substituted or unsubstituted C₁-C₆ alkyl, preferably linear or branched substituted or unsubstituted C₁-C₄ alkyl, more preferably —CH₃, —C₃H₇, —CH(CH₃)₂ or —C(CH₃)₃, substituted or unsubstituted heterocyclyl, preferably unsubstituted morpholinyl or N-substituted piperazinyl, wherein N-substituted piperazinyl is substituted with linear or branched C₁-C₄ alkyl, preferably with —CH₃, or phenyl is partially or fully substituted with —OH or —N(R³²R³³), wherein R³² and R³³ represent independently of each other —H or linear or branched C₁-C₄ alkyl, preferably —H or —CH₃, more preferably —H.

In yet another preferred embodiment, R⁶ in the compounds according to the general formula (I) represents substituted or unsubstituted heteroaryl, wherein the heteroaryl is selected from the group comprising:

Pyrrolyl, thiophenyl, furanyl, imidazolyl, pyrazolyl, thiazolyl, oxazolyl, isothioazolyl, isoxazolyl, pyridinyl, pyrimidinyl, pyrazinyl, pyradizinyl, tetrahydroquinolinyl, quinolinyl, isoquinolinyl, benzoimidazolyl, benzothiazolyl, benzooxazolyl, benzo [1,3] dioxolyl, indolyl, benzofuranyl, benzothiophenyl, indazolyl or chrom-2-onyl,

preferably R⁶ is selected from the group consisting of: imidazolyl, wherein preferably one N-atom of the imidazolyl, is substituted with linear or branched substituted or unsubstituted C₁-C₄ alkyl, more preferably with —CH₃, pyridinyl, preferably 4-pyridinyl, tetrahydroquinolinyl, quinolinyl, benzoimidazolyl, benzothiazolyl, benzo [1,3] dioxolyl, indolyl, indazolyl or chromen-2-onyl.

In yet another preferred embodiment, R⁶ in the compounds according to the general formula (I) represents substituted or unsubstituted heterocyclyl, wherein heterocyclyl is selected from the group comprising:

Aziridinyl, azetidinyl, pyrrolidinyl, piperidinyl, piperazinyl, morpholinyl, preferably R⁶ is selected from azetidinyl, pyrrolidinyl, preferably 2-pyrrolidinyl or 2-piperidinyl, 3-piperidinyl or 4-piperidinyl, preferably 2-piperidinyl.

It is especially preferred within the embodiment described above, that R⁶ in the compounds according to the general formula (I) represents partially or fully substituted heterocyclyl, preferably partially or fully substituted piperidinyl, more preferably N-substituted piperidinyl, substituted with linear or branched substituted or unsubstituted C₁-C₄ alkyl, preferably —CH₃, or —N—COOR³⁴, wherein R³⁴ represents —H or linear or branched substituted or unsubstituted C₁-C₄ alkyl, preferably —(CCH₃)₃.

In yet another preferred embodiment, R⁶ in the compounds according to the general formula (I) represents substituted or unsubstituted C₃-C₈ cycloalkyl, preferably substituted or unsubstituted cyclopentyl or cyclohexyl, and wherein cyclopentyl or cyclohexyl are partially or fully substituted with linear or branched substituted or unsubstituted C₁-C₆ alkyl, —OH, —NH₂ or —NH—COOR³⁵, wherein R³⁵ represents —H or linear or branched substituted or unsubstituted C₁-C₆ alkyl, preferably linear or branched C₁-C₄ alkyl, more preferably —C(CH₃)₃, and wherein cyclopentyl or cyclohexyl are preferably substituted with —NH₂, and wherein cyclopentyl or cyclohexyl are preferably mono-, di- or trisubstituted, more preferably monosubstituted.

In yet another preferred embodiment, R⁶ in the compounds according to the general formula (I) represents —(CH₂)ₚ-Z, wherein p is selected to be 1 or 2 and Z is selected from the group comprising: Substituted or unsubstituted phenyl, wherein in case phenyl is substituted, it is substituted with linear or branched substituted or unsubstituted C₁-C₄ alkyl, preferably —CH₃, substituted or unsubstituted heterocyclyl, preferably substituted or unsubstituted piperidinyl, more preferably N-substituted or unsubstituted 2-piperidinyl, wherein in case 2-piperidinyl is N-substituted, it is substituted with —COOR³⁶, wherein R represents linear or branched substituted or unsubstituted C₁-C₆ alkyl, preferably linear or branched C₁-C₄ alkyl, more preferably —C(CH₃)₃, or Z represents —N(R⁷R⁸), wherein R⁷ and R⁸ represent independently of each other —H, or linear or branched C₁-C₄ alkyl, preferably —H, —CH₃ or —C₂H₅, or R⁶ represents —(CH₂)ₚ-Z, wherein p is selected to be an integer from 0 to 2 and Z is selected to be —(CR⁹R¹⁰R¹¹), wherein R⁹, R¹⁰ and R¹¹ are independently of each other selected from the group consisting of:

—H, linear or branched substituted or unsubstituted C₁-C₅ alkyl, preferably —CH₃,—CH(CH₃)₂, or —CH(CH₃)—C₂H₅, substituted or unsubstituted aryl, or —N(R¹²R¹³), wherein R¹² and R¹³ represent independently of each other —H or linear or branched substituted or unsubstituted C₁-C₄ alkyl, preferably —H or —CH₃.

In a further preferred embodiment, m in the compounds according to the general formula (I) is selected to be 1, R¹, R² and R⁴ represent —H, R³ represents monosubstituted phenyl, R⁵ represents monosubstituted or unsubstituted phenyl, L is selected from the group comprising: —NH—CO—, —NH—SO₂—, —SO₂—NH—, —CO—NH— or —SO₂—, and R⁶ is selected from the group consisting of: —H, linear or branched substituted or unsubstituted C₁-C₄ alkyl, monosubstituted phenyl, substituted or unsubstituted heterocyclyl, wherein heterocyclyl is preferably selected from azetidinyl, pyrrolidinyl, piperidinyl, piperazinyl or morpholinyl, substituted or unsubstituted heteroaryl, wherein heteroaryl is selected from imidazolyl, pyridinyl, tetrahydroquinolinyl, quinolinyl, benzoimidazolyl, benzothiazolyl, benzo [1,3] dioxolyl, indolyl, indazolyl or chromen-2-only or R⁶ represents substituted or unsubstituted C₃-C₈ cycloalkyl.

Especially preferred compounds of general formula (I) are represented by the following subformula

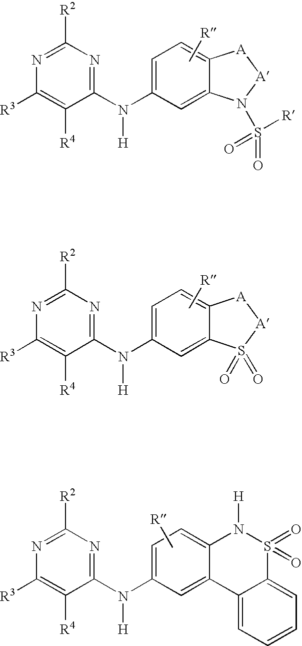

wherein A-A* represents —CH₂—CH₂—, —CH═CH—, —NH—CH₂—, —CH₂—NH—, —N═CH—, —CH═N—, —N═N—, R* is a substituted or unsubstituted aryl, linear or branched substituted or unsubstituted alkyl, substituted or unsubstituted heteroaryl, substituted or unsubstituted heterocyclyl, substituted or unsubstituted cycloalkyl, C₂-C₆ alkenyl or C₂-C₆ alkinyl,

R** represents hydrogen, linear or branched substituted or unsubstituted alkyl or an substitutent selected from Sub.

R², R³, and R⁴ have the meanings as defined above.

Preferably R* is substituted or unsubstituted C₁-C₆ alkyl and most preferably methyl. R³ represents preferably phenyl, substituted or unsubstituted aryl, or substituted or unsubstituted heteroaryl and especially an alkoxy substituted phenyl.

In yet another preferred embodiment, compounds according to the general formula (I) are chiral compounds. It is to be understood, that chiral compounds according to the present invention represent a racemate, or a S or a R enantiomer or a mixture of isomers, respectively.

As used herein, the term “substituent”, or “Sub” or the possibility that one residue may be further substituted with another group refers to the following list of substituents which may be present independently of each other:

—H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH3, —COOC₂H₅, —COOC3H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-CyCIO—C₃H₅, —SOCH(CH₃)₂, —SOC(CH₃)₃, —S0₂CH₃, —SO₂C₂H₅, —S0₂C₃H₇, —SO₂—CyCIO—C₃H₅, —SO₂CH (CH₃)₂, —SO₂C(CH₃ 3, —SO₃H, —SO₃CH₃, —SO₃C₂H₅, —SO₃C₃H₇, —SO₃-cyclo-C₃H₅, —SO₃CH (CH₃) Z, —SO₃C(CH₃)₃, —OCF₃, —OC₃F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N (cyclo-C₃H₅)₂, —NH—CO—N[CH(CH₃)₂]2, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH [CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂—NH—CS—N[C(CH₃)₃]₂, —NH—C (═NH)—NH₂, —NH—C{═NH}—NHCH₃, —NH—C{═NH}—NHC₂H₅, —NH—C(═NH)—NHC₃H₇, —NH—C(═NH)—NH-cyclo-C₃Hs, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N [CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, -0-CO—OCH(CH₃)₂, —O—CO—OC(CH₃)₃, —CH₂F —CHF₂, —CF₃, —CH₂Cl, —CHCl₂, —CCl₃, —CH₂Br, —CHBr₂, —CBr₃, —CH₂, —CH₂, —Cl₃, —CH₂—CH₂F —CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₂—CH₂I—CH₂—CHI₂, —CH₂—Cl₃, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃,-Ph, —H₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH.

In a particularly preferred embodiment of the present invention, the 4,6-disubstituted aminopyrimidine derivative has the structure

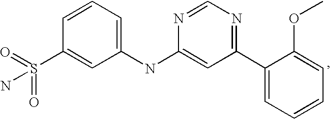

wherein said compound is hereinafter denoted as compound A.

In another particularly preferred embodiment, the 4,6-disubstituted aminopyrimidine derivative has the structure

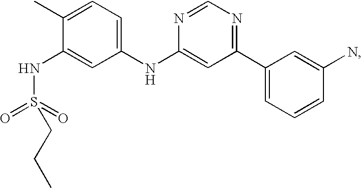

wherein said compound is hereinafter denoted compound B.

In a preferred embodiment, compound A and compound B inhibit CDK9. Preferred 4,6-disubstituted aminopyrimidine derivatives, the inhibitory effects on CDK2, CDK4 and CDK9 of which are known from WO 2005/026129, are the following compounds:
Compound 1: N-{4-[6-(4-Methoxy-phenylpyrimidin-4-yamino]-phenyl}-4-methyl-benzenesulfonamide,
Compound 2: N-{4-[6-(3-Methoxy-phenyl)-pyrimidin-4ylamino]-phenyl}-4-methyl-benzenesulfonamide,
Compound 3: N-{5-[6-(4-Methoxy-phenyl)pyrimidin-4ylamino]-2-methyl-phenyl}methanesulfonamide,
Compound 4: 4-Amino-N-{4-[6-(2-benzyloxy-phenyl)-pyrimidin-4-ylamino]-phenyl}benzamide,
Compound 5: N-{4-[6-(2-Methoxy-phenyl)-pyrimidin-4ylamino]-phenyl}-4-methyl-benzenesulfonamide,
Compound 6: 4-Amino-N-{4-[6-(4-methoxy-phenyl)-pyrimidin-4-ylamino]-phenyl}-benzamide,
Compound 7: [6-(2-Benzyloxy-phenyl)-pyrimidin-4-yl]-(2-pyridin-4-yl-ethyl)-amine,
Compound 8: 4-Amino-N-{4-[(2-methoxy-phenyl)-pyrimidin-4-ylamino]-phenyl}benzamide,
Compound 9: 1-{4-[6-(2-Methoxy-phenyl)-pyrimidin-4-ylamino]-phenyl}-pyrrolidin-2-one,
Compound 10: N-{4-[6-(2-methoxy-phenyl)-pyrimidin-4-ylamino]-phenyl}-acetamide,
Compound 11: N-{4-[6-(4-Hydroxy-phenyl)-pyrimidin-4-ylamino]-phenyl}-4-methyl-benzenesulfonamide,
Compound 12: N-{5-[6-(3-Amino-phenyl)-pyrimidin-4-ylamino]-2-methyl-phenyl}-methanesulfonamide,
Compound 13: [6-(3-Amino-phenyl)-pyrimidin-4-yl]-(2-pyridin-4-yl-ethyl)-amine,
Compound 14: 4-[6-(2-Methoxy-phenyl)-pyrimidin-4-ylamino]l-benzamide,
Compound 15: 4-[6-(2-Methoxy-phenyl)-pyrimidin-4-yamino]l-benzoic acid methyl ester,
Compound 16: 4-Amino-N-{4-[6-(4-hydroxy-phenyl)-pyrimidin-4-ylamino]-phenyl}-benzamide,
Compound 17: 3-(4-{6-[4-Toluene-4-sulfonylamino)-phenylamino]-pyrimidin-4-yl}-phenyl)-propionic acid,
Compound 18: N-{4-[6-(2-Methoxy-phenyl)-pyrimidin-4ylamino]-phenyl}-4-methyl-N-propyl-benzenesulfonamide,
Compound 19; N-{4-[6-(2-Methoxy-phenyl)-pyrimidin-4-ylamino]-phenyl}-2,2-dimethyl-propionamide,
Compound 20: 2-Amino-N-{4-[6-(2-methoxy-phenyl)-pyrimidin-4-ylamino]-phenyl}benzamide,
Compound 21: 4-Amino-N-{4-[6-(3-amino-phenyl)pyrimidin-4-ylamino]L phenyl}-benzamide,
Compound 22: N-[6-(2-Methoxy-phenyl)-pyrimidin-4-yl]-benzene-1,4-diamine,
Compound 23: 4-Isopropyl-N-{4-[6-(2-methoxy-phenyl)-pyrimidin-4-ylamino]-phenyl}-benzenesulfonamide,
Compound 24: N-[4-(6-Chloro-pyrimidin-4ylamino)phenyl]-4-methyl-benzenesulfonamide,
Compound 25: 4-Amino-N-[4-(6-chloro-pyrimidin-4ylamino)-phenyl]-benzamide,
Compound 26: N-[6-(2-Methox-phenyl)-pyrimidin-4-ylamino]-N-methyl-benzene-1,4-diamine,
Compound 27: [{4-[6-(4-Hydroxy-phenyl)-pyrimidin-4-ylamino]-phenyl}-(toluene-4-sulfonyl)-amino]-acetic acid methyl ester,
Compound 28: [{4-[6-(2-Methoxy-phenyl)-pyrimidin-4-ylamino]-phenyl}-(toluene-4-sulfonyl)-amino]-acetic acid methyl ester,
Compound 29: (S)-2-{4-[6-(2-Methoxy-phenyl)-pyrimidin-4-ylamino]-phenylcarbamoyl}-piperidine-1-carboxylic acid tert-butyl ester,
Compound 30: (S)-Piperidine-2-carboxylic acid N-{4-[6-(2-methoxy-phenyl)-pyrimidin-4-ylamino]-phenyl}-amide
Compound 31: 4-Amino-N-{4-[6-(2,4-dimethoxy-phenyl)-pyrimidin-4-ylamino]-phenyl}-benzamide,
Compound 32: 4-Amino-N-{4-[6-styryl-pyrimidin-4-ylamino]-phenyl}-benzamide,
Compound 33: N-{4-[6-(2-Methoxy-phenyl)-pyrimidin-4yl-amino]-phenyl}-methanesulfon-amide,
Compound 34: Biphenyl-4-sulfonic acid-{4-[6-(2-methoxy-phenyl)-pyrimidin-4ylamino]-phenyl}-amide,
Compound 35: 4-Amino-N-{4-[6-(5-isopropyl-2-methoxy-phenyl)-pyrimidin-4-ylamino]-phenyl}-benzamide,
Compound 36: Bicyclo[2.2.1)heptane-2-carboxylic acid {4-[6-(2-methoxyphenyl)-pyrimidin-4-ylamino]-phenyl}-amide,
Compound 37: N-{4-[6-(2-Methoxy-phenyl)-pyrimidin-4-ylamino]-phenyl}-3-methyl-2-phenyl-butyramide,
Compound 38: 1-Cyclohexyl-3-{4-[6-(2-methoxy-phenyl)-pyrimidin-4-ylamino]-phenyl}-urea,
Compound 39: 4-Amino-N-{4-[6-(5-chloro-2-methoxy-phenyl)-pyrimidin-4-ylamino]-phenyl}-benzamide,
Compound 40: E-3-(3-{6-[4-(Toluene-4-sulfonylamino)-phenylamino]-pyrimidin-4-yl}-phenyl)-acrylic acid,
Compound 41: Cyclohexanecarboxylic acid {4-[6-(2-methoxy phenyl)-pyrimidin-4-ylamino]-phenyl}-amide,
Compound 42: N-{4-[6-(2-Methoxy-phenyl)-pyrimidin-4-ylamino]-phenyl}-3,3-dimethyl-butyramide,
Compound 43: 4-Amino-N-{4-[6-(cyclohexylmethyl-amino)-pyrimidin-4-ylamino]-phenyl}-benzamide,
Compound 44: N-Cyclohexyl-{4-[6-(2-methoxy-phenyl)-pyrimidin-4-ylamino]-benzamide,
Compound 45: 4-tert-Butyl-N-{4-[6-(2-methoxy-phenyl)-pyrimidin-4-ylamino]-phenyl}-benzamide,
Compound 46: 2-Dimethylamino-N-{4-[6-(2-methoxy-phenyl)-pyrimidin-4-ylamino]-phenyl}-acetamide,
Compound 47: (1-{4-[6-(2-methoxy-phenyl)pyrimidin-4-ylamino]-phenyl-carbamoyl-cyclopentyl)-carbamic acid tert butyl ester
Compound 48: 2-({4-[6-(2-Methoxy-phenyl)-pyrimidin-4-ylamino]-phenylcarbamoyl}-methyl)piperidine-1-carboxylic acid tert-butyl ester,
Compound 49: N-{4-[6-(2-Methoxy-phenyl)-pyrimidin-4-ylamino]-phenyl}-4-(4-methyl-piperazin-1-yl)-benzamide,
Compound 50: N-{4-[6-(2-Methoxy-phenyl)pyrimidin-4-ylamino]-phenyl}-isonicotinamide,
Compound 51: 4-Amino-N-{4-[6-(2,6-dimethoxy-phenyl)-pyrimidin-4-ylamino]-phenyl}-benzamide,
Compound 52: {4-[6-(2-Methoxy-phenyl)-pyrimidin-4-ylamino]-N-phenyl-benzamide,
Compound 53: N-{4-[6-(2-Methoxy-phenyl)-pyrimidin-4-ylamino]-phenyl}-guanidine,
Compound 54: N-tert-Butyl-4-[6-(2-methoxy-phenyl)-pyrimidin-4-ylamino]-benzamide,
Compound 55: 4-Amino-N-{4-[6-(2-ethoxy-phenyl)-pyrimidin-4-ylamino]-phenyl}-benzamide,
Compound 56: 4-Amino-N-{4-[6-(2,3-dimethoxy-phenyl)-pyrimidin-4-ylamino]-phenyl}-benzamide,
Compound 57: 4-Amino-N-{4-[6-(2,5-dimethoxy-phenyl)pyrimidin-4-ylamino]-phenyl}-benzamide,
Compound 58: 4-Amino-N-{4-[6-(2-isopropoxy-phenyl)-pyrimidin-4-ylamino]-phenyl}-benzamide,
Compound 59: N-{4-[6-(2-Methoxy-phenyl)-pyrimidin-4-ylamino]-phenyl}-2-piperidine-2-yl-acetamide,
Compound 60: 4-Amino-N-{4-[6-(2-hydroxy-ethylamino)-pyrimidin-4-ylamino]-phenyl}-benzamide,
Compound 61: Adamantane-1-carboxylic acid-{4-[6-(2-methoxyphenyl)-pyrimidinyl-amino]-phenyl}-amide,
Compound 62: (4-Benzoxazol-2-yl-phenyl-[6-(2-methoxy-phenyl)-pyrimidin-4yl]-amine,
Compound 63: [4-(1H-Benzimidazole-2-yl)-phenyl]-[6-(2-methoxy-phenyl)-pyrimidin-4-yl]-amine,
Compound 64: 3-Diethylamino-N-{4-[6-(2-methoxy-phenyl)-pyrimidin-4-ylamino]-phenyl}-propion amide,
Compound 65: (S)-1,2,3,4-Tetrahydro-isoquinoline-3-carboxylic acid-{4-[6-(2-methoxy-phenyl)-pyrimidin-4-ylamino]phenyl}amide,
Compound 66: 1-Amino-cyclohexane carboxylic acid-{4-[6-(2-methoxy-phenyl-pyrimidin-4-ylamino]-phenyl}-amide,
Compound 67: 4-Amino-N-[4-(6-pyridin-4-yl-pyrimidin-4-ylamino)-phenyl]-benzamide,
Compound 68: 1-Amino-cyclopentanecarboxylic acid {4-[6-(2-methoxy-phenyl)-pyrimidin-4-ylamino]-phenyl}-amide,
Compound 69: (R)-Piperidine-2-carboxylic acid ({4-[6-(2-methoxy-phenyl)-pyrimidin-4-ylamino]-phenyl}amide,
Compound 70: 1-Methyl-4H-imidazole-4-carboxylic acid {4-[6-(2-methoxy-phenyl)-pyrimidin-4-ylamino]-phenyl}-amide,
Compound 71: N-{4-[6-(2-Methoxy-phenyl)pyrimidin-4-ylamino]-phenyl}-2-phenyl-actetamide,
Compound 72: N-[4-(6-Chloropyrimidin-4-ylamino)-phenyl]-2,2-dimethyl-propionamide,
Compound 73: 2,2-Dimethyl-N-[4-(6-pyridin-3-yl-pyrimidin-4-ylamino)-phenyl]-propionamide,
Compound 74: 2,2-Dimethyl-N-{4-[6-(1-methyl-piperidin-4-ylamino)-pyrimidin-4-ylamino]-phenyl}-propionamide,
Compound 75: 3-{6-[4-(2,2-Dimethyl-propionylamino)phenylamino]-pyrimidin-4-yl}-benzoic acid,
Compound 76: 4-Amino-N-[4-(6-phenyl-pyrimidin-4-ylamino)-phenyl]-benzamide,
Compound 77: 4-Amino-N-[4-(6-thiophen-2-yl-pyrimidin-4-ylamino)-phenyl]-benzamide
Compound 78: 2,2-Dimethyl-N-{4-[6-(4-methyl-piperazin-1-ylamino)-pyrimidin-4-ylamino]-phenyl}-propionamide,
Compound 79: N-{4-[6-(2-Amino-ethylamino)-pyrimidin-4-ylamino]-phenyl}-2,2-dimethyl-propionamide,
Compound 80: N-{-4-[6-(3-Hydroxy-propylamino)-pyrimidin-4-ylamino]-phenyl}-2,2-dimethyl-propionamide,
Compound 81: (S)-2-Amino-N-{4-[6-(2-methoxy-phenyl)-pyrimidin-4-ylamino]-phenyl}-2-phenyl-acetamide,
Compound 82: (S)—N-{4-[6-(2-methoxy-phenyl)-pyrimidin-4-ylamino]-phenyl}-2-methylamino-2-phenyl-acetamide,
Compound 83: (R,R)/(SS)—N-(2-Amino-cyclohexyl)-4-[6-(2-methoxy-phenyl)-pyrimidin-4-ylamino]-benzamide,
Compound 84: Benzothiazole-2 carboxylic acid -{4-[6-(2-methoxy-phenyl)-pyrimidin-4-ylamino]-phenyl}-amide,
Compound 85: N-{4-[6-(2-Benzyloxy-phenyl)-pyrimidin-4-ylamino]-phenyl}-2,2-dimethyl-propionamide,
Compound 86: 4-[6-(2-Methoxy-phenyl)-pyrimidin-4-ylamino]-N-piperidin-3-yl-benzamide.
Compound 87: 1-Methyl-piperidine-3-carboxylic acid {4-[6-(2-methoxy-phenyl)-pyrimidin-4-ylamino]-phenyl}-amide.
Compound 88: 4-(6-Chloro-pyrimidin-4-ylamino)-N-cyclohexyl-benzamide,
Compound 89: 1-Methyl-piperidine-4-carboxylic acid {4-[6-(2-methoxy-phenyl)-pyrimidin-4-yl]-amino]-phenyl}-amide,
Compound 90: (S)-Azetidine-2-carboxylic acid {4-[6-(2-methoxy-phenyl)-pyrimidin-4-yl]-amino]-phenyl}-amide,
Compound 91: (R)-Pyrrolidine-2-carboxylic acid {4-[6-(2-methoxy-phenyl)-pyrimidin-4-yl]-amino]-phenyl}-amide,
Compound 92: [6-(4-Methoxy-phenyl)-pyrimidin-4-yl]-(2-pyridin-4-yl-ethyl)-amine,
Compound 93: [6-(2-Methoxy-phenyl)-pyrimidin-4-yl]-(2-pyridin-4-yl-ethyl)-amine,
Compound 94: 2-[6-(2-Pyridin-4-yl-ethylamino)-pyrimidin-4-yl]-phenol,
Compound 95: 4-[6-(2-Benzyloxy-phenyl)pyrimidin-4-ylamino]-benzamide,
Compound 96: N-(4-{[6-(2-Methoxy-phenyl)-pyrimidin-4-yl]-methyl-amino}-phenyl)-4-methyl-benzenesulfonamide,
Compound 97: 4-Amino-N-{4-[2-amino-6-(2-methoxy-phenyl)-pyrimidin-4-ylamino]-phenyl}-benzamide,
Compound 98: Quinoline-2-carboxylic acid {4-[6-(2-methoxy-phenyl)pyrimidin-4-ylamino]-phenyl}-amide,
Compound 99: 6-(2-Isopropoxy-phenyl)-pyrimidin-4-yl]-(2-pyridin-4-yl-ethyl)-amine,
Compound 100: N-{5-[6-(3-Methoxy-phenyl)-pyrimidin-4-ylamino]-2-methyl-phenyl}-methane sulfonamide,
Compound 101: 2-dimethylamino-N-{4-[6-(2-methoxy-phenyl)-pyrimidin-4-ylamino]-phenyl}-2-phenyl-acetamide,
Compound 102: 3-Amino-N-{4-[6-(2-methoxy-phenyl)-pyrimidin-4-ylamino]-phenyl}propion-amide,
Compound 103: 4-Amino-N-(4-{6-[2-(3-amino-propoxy)-phenyl]-pyrimidin-4-ylamino}-phenyl)-benzamide,
Compound 104: N-{3-[6-(3-Methanesulfonylamino-4-methyl-phenylamino)-pyrimidin-4-yl]-phenyl}-acetamide,
Compound 105: N-{5-[6-(3-Hydroxy-phenyl)-pyrimidin-4-ylamino]-2-methyl-phenyl}methane-sulfonamide,
Compound 106: N-[2-Methyl-5-(6-phenyl-pyrimidin-4-ylamino)-phenyl]-methanesulfonamide,
Compound 107: N-{2-Methyl-5-[6-(3-trifluoromethyl-phenyl)-pyrimidin-4-ylamino]-phenyl}-methanesulfonamide,
Compound 108: N-{5-[6-(3-Methanesulfonylamino-phenyl)-pyrimidin-4-ylamino]-2-methyl-phenyl}-methanesulfonamide,
Compound 109: N-{-[6-(3-Amino-phenyl)-pyrimidin-4-ylamino]-2-methyl-phenyl}-benzene-sulfonamide,
Compound 110: N-[5-([4,5′]Bipyrimidinyl-6-ylamino)-2-methyl-phenyl]-methanesulfonamide,
Compound 111: 1-Benzo [1,3] dioxol-5-yl-3-{4-[6-(2-methoxy-phenyl)-pyrimidin-4-ylamino]-phenyl}-urea,
Compound 112: 1-{4-[6-(2-Methoxy-phenyl)-pyrimidin-4-ylamino]-phenyl}-3-(4-methyl-benzyl)-urea,
Compound 113: 1-tert-Butyl-3-{4-[6-(2-methoxy-phenyl)-pyrimidin-4-ylamino]-phenyl}-urea,
Compound 114: 2,2-Dimethyl-N-{4-[6-(2-trifluoromethyl-phenyl)-pyrimidin-4-ylamino]-phenyl}-propionamide,
Compound 115: 3-[6-(2-Methoxy-phenyl)-pyrimidin-4-ylamino]-benzamide,
Compound 116: Propane-1-sulfonic acid {5-[6-(3-aminophenyl)-pyrimidin-4-ylamino]-2-methyl-phenyl}-amide.
Compound 117: 4-[6-(3-Amino-phenyl)-pyrimidin-4-ylamino]-benzenesulfonamide,
Compound 118: N-{4-[6-(2-Methoxy-phenylpyrimidin-4-ylamino]-phenyl}-2-methyl-2-methylamino-propionamide,
Compound 119: N-{4-[6-(2-Methoxy-phenyl)-pyrimidin-4-ylamino]-3-methyl-phenyl}-2,2-dimethyl-propionamide,
Compound 120: N-{5-[6-(3-Amino-phenyl)-pyrimidin-4-ylamino]-2-benzyloxy-phenyl}-methanesulfonamide,
Compound 121: N-{3-[6-(3-Amino-phenyl)-pyrimidin-4-ylamino]-phenyl}-methanesulfon-amide,
Compound 122: N-{3-[6-(2-Methoxy-phenyl)-pyrimidin-4-ylamino]-phenyl}-2,2-dimethyl-propionamide,
Compound 123: N*1*-[6-(2-Methoxy-phenyl)-pyrimidin-4-yl]-2-methyl-benzene-1,4-diamine,
Compound 124: N-[6-(2-Methoxy-phenyl)-pyrimidin-4-yl]-benzene-1,3-diamine,
Compound 125: 4-[6-(2-Methoxy-phenyl)-pyrimidin-4-ylamino]-N-(4-morpholin-4-yl-phenyl)-benzamide,
Compound 126: 2,2-Dimethyl-N-{4-[6-(2-vinyl-phenyl)-pyrimidin-4-ylamino]-phenyl}propionamide,
Compound 127: N-{4-[6-(2-Fluoro-phenyl)-pyrimidin-4-ylamino]-phenyl}-2,2-dimethyl-propionamide,
Compound 128: (S)-Piperidine-2-carboxylic acid {3-[6-(2-methoxy-phenyl)-pyrimidin-4-ylamino]-phenyl}amide,
Compound 129; 2-Oxo-2H-chromene-3-carboxylic acid {4-[6-(2-methoxy-phenyl)-pyrimidin-4-ylamino]-phenyl}-amide,
Compound 130: Benzo [1,3] dioxole-5-carboxylic acid {4-[6-(2-methoxy-phenyl)-pyrimidin-4-ylamino]-phenyl}-amide,
Compound 131: N-{4-[6-(2-Ethyl-phenyl)-pyrimidin-4-ylamino]-phenyl}-2,2-dimethyl-propion-amide,
Compound 132: N-[4-(6-Biphenyl-2-yl-pyrimidin-4-ylamino)-phenyl]-2,2-dimethyl-propion-amide,
Compound 133; 1H-Indole-3-carboxylic acid {4-[6-(2-methoxy-phenyl)-pyrimidin-4-ylamino]-phenyl}-amide,
Compound 135: N-((1R,2R)/(1S,2S-2-Hydroxy-cyclohexyl)-4-[6-(2-methoxy-phenyl)-pyrimidin-4-ylamino]-benzamide,
Compound 136: N-(4-Hydroxy-phenyl)-4-[6-(2-methoxy-phenyl)-pyrimidin-4-ylamino]-benzamide,
Compound 137: N-(4-Isopropyl-phenyl)-4-[6-(2-methoxy-phenyl)-pyrimidin-4-ylamino]-benzamide,
Compound 138: 1H-Benzoimidazole-5-carboxylic acid {4-[6-(2-methoxy-phenyl)-pyrimidin-4-ylamino]-phenyl}-amide,
Compound 139: 1-Hydroxy-naphthalene-2-carboxylic acid {4-[6-(2-methoxy-phenyl)-pyrimidin-4-ylamino]-phenyl}amide,
Compound 140: (2S,3S)-2-Amino-3-methyl-pentanoic acid {4-[6-(2-methoxy-phenyl)-pyrimidin-4-ylamino]-phenyl}-amide,
Compound 141: 1H-Indazole-3-carboxylic acid {4-[6-(2-methoxy-phenyl)-pyrimidin-4-ylamino]-phenyl}-amide,
Compound 142: Quinoline-8-sulfonic acid {5-[6-(3-aminophenyl)-pyrimidin-4-ylamino]-2-methyl-phenyl}-amide,
Compound 143: (S)-2-Amino-N-{4-[6-(2-methoxy-phenyl)-pyrimidin-4-ylamino]-phenyl}-3-methyl-butyramide,
Compound 144: 1-Methyl-1H-imidazole-4-sulfonic acid {5-[6-(3-amino-phenyl)pyrimidin-4-ylamino]-2-methyl-phenyl}-amide,
Compound 145: 3-Hydroxy-naphthalene-2 carboxylic acid {4-[6-(2-methoxy-phenyl)pyrimidin-4-ylamino]-phenyl}-amide,
Compound 146: 2-Amino-N-{4-[6-(2-methoxy-phenyl)-pyrimidin-4-ylamino]-phenyl}-2-naphthalen-2-yl-acetamide,
Compound 147: {4-[6-(2-Methoxy-phenyl)-pyrimidin-4-ylamino]-phenyl}-morpholin-4-yl-methanone,
Compound 148: N-((1S,2R)/(1R,2S)-2-Aminocyclohexyl)-4-[6-(2-methoxy-phenyl)-pyrimidin-4-ylamino]-benzamide,
Compound 149: 4-Amino-N-{4-[6-(2-methoxy-phenyl)-5-methyl-pyrimidin-4-ylamino]-phenyl}-benzamide,
Compound 150: 3-[6-(2-Methoxy-phenyl)-pyrimidin-4-ylamino]-benzenesulfonamide,
Compound 151: 4-Amino-N-{4-[6-(2-hydroxy-phenyl)-pyrimidin-4-ylamino]-phenyl}-benzamide,
Compound 152: N-[6-(2-Methoxy-phenyl)-5-methyl-pyrimidin-4-yl]-benzene-1,4-diamine,
Compound 153: Propane-2-sulfonic acid {4-[6-(2-methoxy-phenyl)-pyrimidin-4-ylamino]-phenyl}-amide,
Compound 154: Propane-1-sulfonic acid {4-[6-(2-methoxy-phenyl)-pyrimidin-4-ylamino]-phenyl}-amide,
Compound 155: N-{4-[6-(2-Methoxy-phenyl)-pyrimidin-4-ylamino]-phenyl}-benzene sulfonamide,
Compound 156: N-{5-[6-(2-Benzyloxy-phenyl)-pyrimidin-4-ylamino]-2-methyl-phenyl}-methanesulfonamide,
Compound 157: N-{5-[6-(3-Dimethylamino-phenyl)-pyrimidin-4-ylamino]-2-methyl-phenyl}-methanesulfonamide,
Compound 158: N-{5-[6-(2-Isopropoxy-phenyl)pyrimidin-4-ylamino]-2-methyl-phenyl}-methanesulfonamide,
Compound 159: N-Bis-propane-1-sulfonic acid-{4-[6-(2-methoxy-phenyl)-5-methyl-pyrimidin-4-ylamino]-phenyl}-amide,
Compound 160: Propane-1-sulfonic acid {4-[6-(2-methoxy-phenyl)-5-methyl-pyrimidin-4-ylamino]-phenyl}-amide,
Compound 161: N-(2-Amino-cyclohexyl)-4-[6-(4-methoxy-phenyl)-pyrimidin-4-ylamino]-benzamide,
Compound 162: N-{5-[6-(2-Methoxy-phenyl)-pyrimidin-4-ylamino]-2-methyl-phenyl}methanesulfonamide,
Compound 163: N-{5-[6-(3-Cyano-phenyl)-pyrimidin-4-ylamino]-2-methyl-phenyl}-methane sulfonamide,
Compound 164: (S)-Piperidine-2-carboxylic acid {3-[6-(2-benzyloxy-phenyl)-pyrimidin-4-ylamino]-phenyl}-amide,
Compound 165: N-{5-[6-(3-Formyl-phenyl)-pyrimidin-4-ylamino]-2-methyl-phenyl}-methane sulfonamide,
Compound 166: N-{5-[6-(2-Hydroxymethyl-phenyl)-pyrimidin-4-ylamino]-2-methyl-phenyl}-methanesulfonamide,
Compound 167; (S)-Piperidine-2-carboxylic acid {3-[6-(4-methoxy-phenyl)-pyrimidin-4-ylamino]-phenyl}-amide,
Compound 168: (S)-Piperidine-2-carboxylic acid {3-[6-(3-formyl-phenyl)-pyrimidin-4-ylamino]-phenyl}-amide,
Compound 169: (S)-Piperidine-2-carboxylic acid {3-[6-(3-dimethylamino-phenyl)-pyrimidin-4-ylamino]-phenyl}-amide,
Compound 170: (S)-Piperidine-2-carboxylic acid {3-[6-(2-hydroxymethyl-phenyl)-pyrimidin-4-ylamino]-phenyl}-amide,
Compound 171: (S)-Piperidine-2-carboxylic acid {3-[6-(2-methoxy-pyridin-3-yl)-pyrimidin-4-ylamino]-phenyl}-amide,
Compound 172: (S)-Piperidine-2-carboxylic acid {3-[6-(6-methoxy-pyridin-3-yl)-pyrimidin-4-ylamino]-phenyl}amide,
Compound 173: (S)-Piperidine-2-carboxylic acid {3-[6-(4-benzyloxy-phenyl)-pyrimidin-4-ylamino]-phenyl}-amide,
Compound 174: (S)-Piperidine-2-carboxylic acid {3-[6-(4-phenoxy-phenyl)-pyrimidin-4-yl-amino]-phenyl}-amide
Compound 175: N-{5-[6-(4-Hydroxymethyl-phenyl)-pyrimidin-4-ylamino]-2-methyl-phenyl}-methanesulfonamide, and
Compound 176: N-{5-[6-(2-Methoxy-pyridin-3-yl)-pyrimidin-4-ylamino]-2-methyl-phenyl}-methanesulfonamide.
Compound 177: (S)-Piperidine-2-carboxylic acid {4-[6-(4-acetylamino-phenyl)-pyrimidin-4-ylamino]-phenyl}-amide,
Compound 178: (S)-Piperidine-2-carboxylic acid {4-[6-(3-methanesulfonylaminophenyl)-pyrimidin-4-ylamino]-phenyl}-amide,
Compound 179: (S)-Piperidine-2-carboxylic acid {4-[6-(3-acetyl-phenyl)-pyrimidin-4-ylamino]-phenyl}-amide,
Compound 180: (S)-Piperidine-2-carboxylic acid {4-[6-(4-cyclopentylcarbamoyl-phenyl)-pyrimidin-4-ylamino]-phenyl}-amide,
Compound 181: N-{5-[6-(2-Hydroxy-phenyl)-pyrimidin-4-ylamino]-2-methyl-phenyl}-methanesulfonamide,
Compound 182: (E)-3-{3-[6-(3-Methanesulfonylamino-4-methyl-phenyl-amino)-pyrimidin-4-yl]-phenyl}-acrylic acid methyl ester,
Compound 183: N-{5-[6-(3-Hydroxymethyl-phenyl)pyrimidin-4-ylamino]-2-methyl-phenyl}-methanesulfonamide,
Compound 184: N-Butyl-3-[6-(2-methoxy-phenyl)-pyrimidin-4-ylamino]-benzenesulfonamide,
Compound 185: (3-Methanesulfonyl-phenyl)-[6-(2-methoxy-phenyl)-pyrimidin-4-yl]-amine,
Compound 186: (S)-Piperidine-2-carboxylic acid {4-[6-(2,3-dimethoxy-phenyl)pyrimidin-4-ylamino]-phenyl}-amide,
Compound 187: (S)-Piperidine-2-carboxylic acid {4-[6-(2,4-dimethoxy-phenyl)-pyrimidin-4-ylamino]-phenyl}amide,
Compound 188: (S)-Piperidine-2-carboxylic acid {4-[6-(2-isopropoxy-phenyl)-pyrimidin-4-ylamino]-phenyl}-amide,
Compound 189: (S)-Piperidine-2-carboxylic acid {4-[6-(2-methylsulfanyl-phenyl)-pyrimidin-4-ylamino]-phenyl}-amide,
Compound 190: (S)-Piperidine-2-carboxylic acid {4-[6-(2-trifluoromethoxy-phenyl)-pyrimidin-4-ylamino]-phenyl}-amide,
Compound 191: (S)-Piperidine-2-carboxylic acid {4-[6-(5-acetyl-thiophen-2-yl)-pyrimidin-4-ylamino]-phenyl}-amide,
Compound 192: (S)-Piperidine-2-carboxylic acid {4-[6-(2-chloro-phenyl)-pyrimidin-ylamino]-phenyl}-amide,
Compound 193: (S)-Piperidine-2-carboxylic acid {4-[6-(3-hydroxymethyl-phenyl)-pyrimidin-4-ylamino]-phenyl}-amide,
Compound 194: N-((1S,2S)/(1R,2R)-2-Amino-cyclohexyl)-4-[6-(3-hydroxy-phenyl)-pyrimidin-4-ylamino]-benzamide,
Compound 195: N-((1S,2S)/(1R,2R)-2-Amino-cyclohexyl)-4-[6-(3-methanesulfonyl-amino-phenyl)-pyrimidin-4-ylamino]-benzamide,
Compound 196: 4-[6-(2-Acetylamino-phenyl)-pyrimidin-4-ylamino]-N-((1S,2S)/(1R,2R-2-amino-cyclohexyl)-benzamide,
Compound 197: N-{5-[6-(2-Methoxymethyl-phenyl)-pyrimidin-4-ylamino]-2-methyl-phenyl}-methane-sulfonamide,
Compound 198: N-((1R,2R)/(1S,2S)-2-Amino-cyclohexyl)-4-[6-(2-benzyloxy-phenyl)-pyrimidin-4-yl-amino]-benzamide,
Compound 199: N-((1R,2R)/(1S,2S)-2-Amino-cyclohex-yl)-4-[6-(2-isopropoxy-phenyl)-pyrimidin-4-ylamino]-benzamide,
Compound 200: 4-Amino-N-(4-[6-(4-methoxy-phenyl)-5-methyl-pyrimidin-4-ylamino]-phenylbenzamide,
Compound 201: 3-{6-[4-((1R,2R)/(1S,2S)-2-Amino-cyclohexylcarbamoyl)-phenylamino]-pyrimidin-4-yl}-benzoic acid methyl ester,
Compound 202: (S)-Piperidine-2-carboxylic acid {4-[6-(4-methoxy-phenyl)-pyrimidin-4-ylamino]-phenyl}-amide,
Compound 203: (S)-Piperidine-2-carboxylic acid {4-[6-(3-methoxymethyl-phenyl)-pyrimidin-4-ylamino]-phenyl}-amide,
Compound 204: N-[6-(2-Methoxy-phenyl)-2-methyl)-pyrimidin-4-yl]-benzene-1,4-diamine,
Compound 205: N-[6-(4-Methoxy-phenyl)-2-methyl-pyrimidin-4-yl]-benzene-1,4-diamine,
Compound 206: N-((1R,2R)/(1S,2S)-2-Amino-cyclohexyl)-4-[6-(3-amino-phenyl)-pyrimidin-4-ylamino]-benzamide,
Compound 207: 4-[6-(3-Acetylamino-phenyl)-pyrimidin-4-ylamino]-N-((1R,2R)/(1S,2S)-2-amino-cyclohexyl)-benzamide,
Compound 208: N-((1R,2R)/(1S,2S)-2-Amino-cyclohexyl)-4-[6-(4-benzyloxy-phenyl)-pyrimidin-4-ylamino]-benzamide,
Compound 209: N-((1R,2R)/(1S,2S)-2-Amino-cyclohexyl)-4-[6-(3-cyano-phenyl)-pyrimidin-4-ylamino]-benzamide,
Compound 210: N-((1R,2R)/(1S,2S)-2-Aminocyclohexyl)-4-[6-(2-methoxymethyl-phenyl)-pyrimidin-4-ylamino]-benzamide,
Compound 211: (S)-Piperidine-2-carboxylic acid {4-[6-(3-dimethylaminomethyl-phenyl)-pyrimidin-4-ylamino]-phenyl}-amide,
Compound 212: N-((1R,2R)/(1S,2S)-2-Amino-cyclohexyl)-4-(6-quinolin-3-yl-pyrimidin-4-ylamino)-benzamide,
Compound 213: N-((1R,2R)/(1S,2S)-2-Amino-cyclohexyl)-4-(2′-methoxy-[4,5′]bipyrimidinyl-6-ylamino)-benzamide,
Compound 214: 3-[6-(3-Amino-phenyl)-pyrimidin-4-ylamino]-benzenesulfonamide,
Compound 215: 3-[6-(4-Methoxy-phenyl)-pyrimidin-4-ylamino]-benzenesulfonamide,
Compound 216: (R,R)—N-(2-Amino-cyclohexyl)-4-[6-(2-hydroxymethyl-phenyl)-pyrimidin-4-ylamino]-benzamide,
Compound 217: N-(2-Diethylamino-ethyl)-4-[6-(2-methoxy-phenyl)-pyrimidin-4-ylamino]-benzamide,
Compound 218: (R,R)—N-(2-Amino-cyclohexyl)-4-[6-(2-hydroxy-phenyl)-pyrimidin-4-ylamino]-benzamide,
Compound 219: (R,R)—N-(2-Amino-cyclohexyl)-4-[6-(2-methoxy-pyridin-3-yl)-pyrimidin-4-ylamino]-benzamide,
Compound 220: (R,R)—N-(2-Amino-cyclohexyl)-4-[6-(5-dimethylaminomethyl-pyridin-3-yl)-pyrimidin-4-ylamino]-benzamide,
Compound 221: (R,R)-5-{6-[4-(2-Amino-cyclohexylcarbamoyl)-phenylamino]-pyrimidin-4-yl}-pyridine-2-carboxylic acid dimethylamide,
Compound 222: (R,R)—N-(2-Amino-cyclohexyl)-4-[6-(6-methylsulfanyl-pyridin-3-yl)-pyrimidin-4-ylamino]-benzamide,
Compound 223: (R,R)—N-(2-Amino-cyclohexyl)-4-[6-(5-aminomethyl-pyridin-3-yl)-pyrimidin-4-ylamino]-benzamide,
Compound 224: (R,R)—N-(2-Amino-cyclohexyl)-4-[6-(4-methylsulfanyl-pyridin-3-yl)-pyrimidin-4-ylamino]-benzamide,
Compound 225: N-(2-Amino-cyclohexyl)-4-[6-(5-hydroxymethyl-pyridin-3-yl)-pyrimidin-4-ylamino]-benzamide,
Compound 226: rac-4-[6-(2-Methoxy-phenyl)-pyrimidin-4-ylamino]-N-pyrrolidin-3-yl-benzamide,
Compound 227: (R,R)—N-(2-Amino-cyclohexyl)-4-[6-(5-dimethyamino-pyridin-3-yl)-pyrimidin-4-ylamino]-benzamide,
Compound 228: (R,R)-4-[6-(5-Acetyl-thiophen-2-yl)-pyrimidin-4-ylamino]-N-(2-amino-cyclohexyl)benzamide,
Compound 229: [6-(2-Methoxy-phenyl)-pyrimidin-4-yl]-[3-(piperidine-1-sulfonyl)-phenyl]-amine N-(2-diethylamino-ethyl)-benzamide,
Compound 230: (R,R)-4-[6-(2-Acetyl-phenyl)pyrimidin-4-ylamino]-N-(2-amino-cyclohexyl)-benzamide,
Compound 231: 4-[6-(2-Methoxy-phenyl)-pyrimidin-4-ylamino]-N-pyridin-3-yl-benzamide,
Compound 232: N-(1-Acetyl-piperidin-3-yl)-4-[6-(2-methoxy-phenyl)-pyrimidin-4-ylamino]-benzamide,
Compound 233: (R,R)—N-(2-Amino-cyclohexyl)-4-[6-(2-dimethylamino-phenyl)pyrimidin-4-ylamino]-benzamide,
Compound 234: 4-{4-[6-(2-Methoxy-phenyl)-pyrimidin-4-ylamino]-benzoylamino}-pyrrolidine-1,2-dicarboxylic acid 1-tert-butyl ester 2-methyl ester,
Compound 235: 2-Chloro-5-[6-(2-methoxy-phenyl)-pyrimidin-4-ylamino]-benzenesulfonamide,
Compound 236: [6-(2-Methoxy-phenyl)-pyrimidin-4-yl]-[3-(piperidine-1-sulfonyl)-phenyl]-amine,
Compound 237: N-Allyl-3-[6-(2-methoxy-phenyl)-pyrimidin-4-ylamino]-benzenesulfonamide
Compound 238: N-Benzyl-3-[6-(2-methoxy-phenyl)-pyrimidin-4-ylamino]-benzenesulfonamide,
Compound 239: [6-(2-Methoxy-phenyl)-pyrimidin-4-yl]-[3-(pyrrolidine-1-sulfonyl)-phenyl]-amine,
Compound 240: [6-(2-Methoxy-phenyl)-pyrimidin-4-yl]-[3-(morpholine-4-sulfonyl)-phenyl]-amine,
Compound 241: 3-[6-(2-Methoxy-phenyl)-pyrimidin-4-ylamino]-N-methyl-benzenesulfonamide,
Compound 242: N-[6-(2-Methoxy-phenyl)-pyrimidin-4-yl]-N-(3-sulfamoyl-phenyl)-acetamide,
Compound 243: N,N-Diallyl-3-[6-(2-methoxy-phenyl)-pyrimidin-4-ylamino]-benzenesulfonamide,
Compound 244: 3-[6-(2-Benzyloxy-phenyl)pyrimidin-4-ylamino]-benzenesulfonamide,
Compound 245: [6-(2-Methoxy-phenyl)-pyrimidin-4-yl]-[4-(4-nitro-benzenesulfonyl)-phenyl]-amine,
Compound 246: [6-(2-Methoxy-phenyl)-pyrimidin-4-yl]-(4-trifluoromethanesulfonyl-phenyl)-amine,
Compound 247: (4-Methanesulfonyl-phenyl)-[6-(2-methoxy-phenyl)-pyrimidin-4-yl]-amine,
Compound 248: N-(3,4-Dimethyl-isoxazol-5-yl)-4-[6-(2-methoxy-phenyl)-pyrimidin-4-ylamino]-benzenesulfonamide,
Compound 249: 4-[6-(2-Methoxy-phenyl)-pyrimidin-4-ylamino]-N-propyl-benzenesulfonamide,
Compound 250: 4-[6-(2-Methoxy-phenyl)-pyrimidin-4-ylamino]-benzenesulfonamide,
Compound 251: 4-[6-(2-Methoxy-phenyl)-pyrimidin-4-ylamino]-N,N-dimethyl-benzenesulfonamide,
Compound 252: N-(2-Methoxy-ethyl)-4-[6-(2-methoxy-phenyl)-pyrimidin-4-ylamino]-benzenesulfonamide,
Compound 253: [6-(2-Benzyloxy-phenyl)-pyrimidin-4-yl]-(3-methanesulfonyl-phenyl)-amine,
Compound 254: 2-[6-(3-Methanesulfonyl-phenylamino)-pyrimidin-4-yl]-phenol,
Compound 255: [6-(3-Amino-phenyl)-pyrimidin-4-yl]-(3-methanesulfonyl-phenyl)-amine,
Compound 256: 5-[6-(2-Methoxy-phenyl)-pyrimidin-4-ylamino]-2-methyl-benzenesulfonic acid,
Compound 257: 2-{3-[6-(2-Methoxy-phenyl)-pyrimidin-4-ylamino]-benzenesulfonyl}-ethanol,
Compound 258: (2-Fluoro-5-methanesulfonyl-phenyl)-[6-(2-methoxy-phenyl)-pyrimidin-4-yl]-amine,
Compound 259: [6-(2-Amino-phenyl)-pyrimidin-4-yl]-(3-methanesulfonyl-phenyl)-amine,
Compound 260: [6-(2-Methoxy-phenyl)-pyrimidin-4-yl]-(3-trifluoromethanesulfonyl-phenyl)-amine,
Compound 261: (3-Methanesulfonyl-phenyl)-[6-(2-Phenoxy-phenyl)-pyrimidin-4-yl]-amine,
Compound 262: [6-(2-Butoxy-phenyl)-pyrimidin-4-yl]-(3-methanesulfonyl-phenyl)-amine,
Compound 263: (3-Ethenesulfonyl-phenyl-[6-(2-methoxy-phenyl)-pyrimidin-4-yl]-amine,
Compound 264: (S)-Piperidine-2-carboxylic acid {4-[6-(4-methylsulfanyl-phenyl)-pyrimidin-4-ylamino]-phenyl}-amide,
Compound 265: 2-Chloro-4-[6-(2-methoxy-phenyl)-pyrimidin-4-ylamino]-benzoic acid methyl ester,
Compound 266: [6-(2-Methoxy-phenyl)-pyrimidin-4-yl]-(4-phenoxy-benzyl)-amine,
Compound 267: 4-[6-(2-Methoxy-phenyl)-pyrimidin-4-ylamino]-3-methyl-benzoic acid methyl ester,
Compound 268: [6-(3-Amino-phenyl)-pyrimidin-4-yl]-(1-methanesulfonyl-2,3-dihydro-1H-indol-6-yl)-amine,
Compound 269: 3-[6-(2-Methoxy-phenyl)-pyrimidin-4-ylamino]-piperidine-1-carboxylic acid tert-butyl ester,
Compound 270: {4-[6-(2-Methoxy-phenyl)pyrimidin-4-ylamino]-phenyl}-acetic acid,
Compound 271: (1H-Indazol-6-yl)-[6-(2-methoxy-phenyl)-pyrimidin-4-yl]-amine,
Compound 272: 1-{4-[6-(2-Methoxy-phenyl)-pyrimidin-4-ylamino]-phenyl}-butan-1-one,
Compound 273: [6-(2-Methoxy-phenyl)-pyrimidin-4-yl]-piperidin-3-yl-amine,
Compound 274: {4-[6-(2-Methoxy-phenyl)-pyrimidin-4-ylamino]-phenyl}-phenyl-methanone,
Compound 275: N-[6-(2-Methoxy-phenyl)-pyrimidin-4-yl]-N′-phenyl-benzene-1,3-diamine,
Compound 276: (3-[1,3]Dioxan-2-yl-phenyl)-[6-(2-methoxy-phenyl)-pyrimidin-4-yl]-amine,
Compound 277: (3-Methoxy-phenyl)-[6-(2-methoxy-phenyl)-pyrimidin-4-yl]-amine,
Compound 278: (4-Methoxy-phenyl)-[6-(2-Methoxy-phenyl)pyrimidin-4-yl]-amine,
Compound 279: N-[6-(2-Methoxy-phenyl)-pyrimidin-4-yl]-N′-phenyl-benzene-1,4-diamine,
Compound 280: [6-(2-Methoxy-phenyl)-pyrimidin-4-yl]-(4-morpholin-4-yl-phenyl)-amine,
Compound 281: (2-Fluoro-phenyl)-[6-(2-methoxy-phenyl)-pyrimidin-4-yl]-amine,
Compound 282: (1-Benzyl-piperidin-4-yl)-[6-(2-Methoxy-phenyl)-pyrimidinyl]-amine,
Compound 283: (4-Butyl-phenyl)-[6-(2-methoxy-phenyl)-pyrimidin-4-yl]-amine,
Compound 284: [6-(2-Methoxy-phenyl)-pyrimidin-4-yl]-(4-phenoxy-phenyl)-amine,
Compound 285: 4-{[6-(2-Methoxy-phenyl)-pyrimidin-4-ylamino]-methyl}-benzenesulfonamide,
Compound 286: rac-1-Dimethylamino-3-{4-[6-(2-methoxy-phenyl)-pyrimidin-4-ylamino]-phenoxy}-propan-2-ol,
Compound 287: N-[6-(4-Methoxy-phenyl)-5-methyl-pyrimidin-4-yl]-benzene-1,4-amine,
Compound 288: N-[6-(3-Amino-phenyl)-5-methyl-pyrimidin-4-yl]-benzene-1,4-amine,
Compound 289: [6-(2-Methoxy-phenyl)pyrimidin-4-yl]-piperidin-4-yl-amine,
Compound 290: 4-[6-(2-Benzyloxy-phenyl)-pyrimidin-4-ylamino]-piperidine-1-carboxylic acid tert-butyl ester,
Compound 291: Cyclohexyl-[6-(2-methoxy-phenyl)-pyrimidin-4-yl]-amine,
Compound 292: 4-{6-[2-(2-Morpholin-4-yl-ethoxy)-phenyl]-pyrimidin-4-ylamino}-benzoic acid methyl ester,
Compound 293: 2-Methoxy-4-[6-(2-methoxy-phenyl)-pyrimidin-4-ylamino]-benzoic acid methyl ester,
Compound 294: {4-[6-(2-Benzyloxy-phenyl)-pyrimidin-4-ylamino]-phenyl}-acetic acid,
Compound 295: [6-(2-Methoxy-phenyl)-pyrimidin-4-yl]-(3-nitro-phenyl)-amine,
Compound 296: {3-[6-(2-Methoxy-phenyl)-pyrimidin-4-ylamino]-phenyl}-methanol,
Compound 297: [6-(2-Benzyloxy-phenyl)-pyrimidin-4-yl]-phenyl-amine,
Compound 298: [6-(2-Methoxy-phenyl)-pyrimidin-4-yl]-phenyl-amine,
Compound 299: (4-Fluorophenyl)-[6-(2-methoxy-phenyl)-pyrimidin-4-yl]-amine,
Compound 300: [6-(2-Methoxy-phenyl)-pyrimidin-4-yl]-(3-phenoxy-phenyl)amine,
Compound 301: [6-(2-Methoxy-phenyl)-pyrimidin-4-yl]-(3-methylsulfanyl-phenyl)-amine,
Compound 302: [6-(2-Benzyloxy-phenyl)-pyrimidin-4-yl]-piperidin-4-yl-amine,
Compound 303: 3-[6-(2-Methoxy-phenyl)-pyrimidin-4-ylamino]-phenol,
Compound 304: 1-{4-[6-(2-Methoxy-phenyl)-pyrimidin-4-ylamino]-phenyl}-ethanone,
Compound 305: 2-Chloro-4-[6-(2-methoxy-phenyl)-pyrimidin-4-yamino]-benzoic acid,
Compound 306: {4-[6-(2-Methoxy-phenyl)-pyrimidin-4-ylamino]-butyl}-carbamic acid tert-butyl ester,
Compound 307: [6-(2-Benzyloxy-phenyl)-pyrimidin-4-yl]-(1-methanesulfonyl-2,3-dihydro-1H-indol-6-yl)-amine,
Compound 308: 4-[6-(2-Methoxy-phenyl)-pyrimidin-4-ylamino]-piperidine-1-carboxylic acid tert-butyl ester,
Compound 309: 4-[6-(2-Amino-phenyl)-pyrimidin-4-ylamino]-benzoic acid methyl ester,
Compound 310: [6-(2-Methoxy-phenyl)-pyrimidin-4-yl]-(4-methylsulfanyl-phenyl)-amine,
Compound 311: N¹-[6-(2-Methoxy-phenyl)-pyrimidin-4-ylamino]-butane-1,4-diamine,
Compound 312: 1-{4-[6-(2-Benzyloxy-phenyl)-pyrimidin-4-ylamino]-phenoxy}-3-dimethylamino-propan-2-ol,
Compound 313: (1-Methanesulfonyl-2,3-dihydro-1H-indol-6-yl)-[6-(2-methoxy-phenyl)-pyrimidin-4-yl]-amine,
Compound 314: N-(2-Amino-cyclohexyl)-4-[6-benzotriazol-1-yloxy)-pyrimidin-4-ylamino]-benzamide,
Compound 315: (2-{4-[6-(Benzotriazol-1-yloxy)-pyrimidin-4-ylamino]-benzoylamino}-cyclohexyl)-carbamic acid tert-butyl ester,
Compound 316: 1-{3-[6-(2-Methoxy-phenyl)-pyrimidin-4-ylamino]-phenyl}-ethanone,
Compound 317: [6-(2-Methoxy-phenyl)-pyrimidin-4-yl]-(4-piperidin-1-yl-phenyl)-amine,
Compound 318: 3-Hydroxy-4-[6-(2-methoxy-phenyl)-pyrimidin-4-ylamino]-benzoic acid methyl ester,
Compound 319: 2-Hydroxy-4-[6-(2-methoxy-phenyl)-pyrimidin-4-ylamino]-benzoic acid methyl ester,
Compound 320: 4-Amino-butane-1-sulfonic acid {5-[6-(2-methoxy-phenyl)-pyrimidin-4-ylamino]-2-methyl-phenyl}-amide,
Compound 321: (3-{6-[3-(4-Amino-butane-1-sulfonylamino)-4-methyl-phenylamino]-pyrimidin-4-yl}-phenyl)-carbamic acid 9H-fluoren-9-ylmethyl ester,
Compound 322: 3-Methoxy-4-[6-(2-methoxy-phenyl)-pyrimidin-4-ylamino]-benzoic acid methyl ester,
Compound 323: 4-{6-[2-(2-Piperidin-1-yl-ethoxy)-phenyl]-pyrimidin-4-ylamino}-benzoic acid methyl ester,
Compound 324: 4-{-6-[2-(2-Dimethylaminoethoxy)-phenyl]-pyrimidin-4-ylamino}-benzoic acid methyl ester,
Compound 325: 4-{6-[2-(2-Diisopropylamino-ethoxy)-phenyl]-pyrimidin-4-ylamino}-benzoic acid methyl ester,
Compound 326: 4-{-6-[2-(2-Diethylamino-ethoxy)-phenyl]-pyrimidin-4-ylamino}-benzoic acid methyl ester,
Compound 327: (S,S)-4-{4-[6-(2-Methoxy-phenyl)-pyrimidin-1-ylamino]-benzoylamino}pyrrolidine-2-carboxylic acid methyl ester,
Compound 328: (S,S)-4-{4-[6-(2-Methoxy-phenyl)-pyrimidin-4-amino]-benzoylamino}-pyrrolidine-2-carboxylic acid,
Compound 329: (S,S)-6-[(4-{4-[6-(2-Methoxy-phenyl)-pyrimidin-4-ylamino]-benzoylamino}-pyrrolidine-2-carbonyl)-amino]-hexanoic acid,
Compound 330: N-Cyclopentyl-4-[6-(2-methoxy-phenyl)-pyrimidin-4-ylamino]-benzamide,
Compound 331: 3-[6-(2-Methoxy-phenyl)-pyrimidin-4-ylamino]-benzenesulfonamide,
Compound 332: (3-Methanesulfonyl-phenyl)-[6-(2-Methoxy-phenyl)-pyrimidin-4-yl]-amine,
Compound 333: 2-{3-[6-(2-Methoxy-phenyl)-pyrimidin-4-ylamino]-benzenesulfoyl}ethanol,
Compound 334: N-4,6-Dimethyl-pyrimidin-2-yl)-4-[6-(2-methoxy-phenyl)-pyrimidin-4-ylamino]-benzenesulfonamide,
Compound 335: 4-[6-(2-Methoxy-phenyl)-pyrimidin-4-ylamino]-N-thiazol-2-yl-benzenesulfonamide,
Compound 336: (1-Benzyl-piperidin-3-yl)[6-(2-methoxy-phenyl)-pyrimidin-4-yl]-amine,
Compound 337: 3-[6-(2-Methoxy-phenyl)-pyrimidin-4-ylamino]-azepan-2-one,
Compound 338: 4-[6-(2-Methoxy-phenyl)-pyrimidin-4-ylamino]-N-phenyl-benzenesulfonamide,
Compound 339: rac-[6-(2-Methoxy-phenyl)-pyrimidin-4-yl]-(1,2,3,4-tetrahydro-naphthalen-1-yl)-amine,
Compound 340: [6-(2-Methoxy-phenyl)-pyrimidin-4-yl]-(2,2,6,6-tetramethyl-piperidin-4-yl)amine,
Compound 341: 4-[6-(2-Methoxy-phenyl)-pyrimidin-4-ylamino]-N-methyl-benzenesulfonamide,
Compound 342: (1,1-Dioxo-1H-1λ⁶-benzo[b]thiophen-6-yl)-[6-(2-methoxy-phenyl)-pyrimidin-4-yl]-amine,
Compound 343: N-Acetyl-4-[6-(2-methoxy-phenyl)-pyrimidin-4-ylamino]-benzenesulfonamide,
Compound 344: N-(2,6-Dimethyl-pyrimidin-4-yl)-4-[6-(2-methoxy-phenyl)-pyrimidin-4-ylamino]-benzenesulfonamide,
Compound 345: [6-(2-Methoxy-phenyl)-pyrimidin-4-yl]-[4-(piperidine-1-sulfonyl)-phenyl]-amine,
Compound 346: 3-{3-[6-(2-Methoxy-phenyl)-pyrimidin-4-ylamino]-phenoxy}-piperidine-1-carboxylic acid tert-butyl ester,
Compound 347: [6-(2-Fluoro-6-methoxy-phenyl)-pyrimidin-4-yl]-(3-methanesulfonyl-phenyl)-amine,
Compound 348: [6-(4-Fluoro-2-methoxy-phenyl)-pyrimidin-4-yl]-(3-methanesulfonyl-phenyl)-amine,
Compound 349: [6-(5-Fluoro-2-methoxy-phenyl)-pyrimidin-4-yl]-(3-methanesulfonyl-phenyl)-amine,
Compound 350: [6-(2-Methoxy-phenyl)-pyrimidin-4-yl]-pyridin-3-yl-amine,
Compound 351: 2-{4-[6-(2-Methoxy-phenyl)-pyrimidin-4-ylamino]-phenyl}-ethanol,
Compound 352: (9,9-Dioxo-9,10-dihydro-9λ⁶-thia-10-aza-phenanthren-3-yl)-[6-(2-methoxy-phenyl)-pyrimidin-4-yl]-amine,
Compound 353: [6-(2-Methoxy-phenyl)-pyrimidin-4-yl]-(1-methyl-1H-indazol-6-yl)-amine,
Compound 354: Benzo [1,2,5]thiadiazol-4-yl-[6-(2-methoxy-phenyl)-pyrimidin-4-yl]-amine,
Compound 355: Benzo [1,2,5]thiadiazol-5-yl-[6-(2-methoxy-phenyl)-pyrimidin-4-yl]-amine,
Compound 356: rac-[6-(2-Methoxy-phenyl)-pyrimidin-4-yl]-[3-(piperidin-3-yloxy)-phenyl]-amine,
Compound 357: [6-(2-Methoxy-phenyl)-pyrimidinyl]-{1-[6-(2-methoxy-phenyl)-pyrimidin-4-yl]-1H-indazol-5-yl}-amine,
Compound 358: (1H-Indol-5-yl)-[6-(2-methoxy-phenyl)-pyrimidin-4-yl]-amine,
Compound 359: (3-Methanesulfinyl-phenyl)-[6-(2-methoxy-phenyl)-pyrimidin-4-yl]-amine,
Compound 360: (1H-Indazol-5-yl)-[6-(2-methoxy-phenyl)-pyrimidin-4-yl]-amine,
Compound 361: 4-[6-(2-Methoxy-phenyl)-pyrimidin-4-ylamino]-thiophene-3-carboxylic acid methyl ester,
Compound 362: 4-Methanesulfonyl-benzyl-[6-(2-methoxy-phenyl)-pyrimidin-4-yl]-amine,
Compound 363: (5-Chloro-1H-indazol-3-yl)-[6-(2-methoxy-phenyl)-pyrimidin-4-yl]-amine,
Compound 364: [6-(2-Methoxy-phenyl)-pyrimidin-4-yl]-(5-methyl-isoxazol-3-yl)amine,
Compound 365: 3-[6-(2-Methoxy-phenyl)-pyrimidin-4-ylamino]-N,N-dimethyl-benzenesulfonamide,
Compound 366: N-Ethyl-3-[6-(2-methoxy-phenyl)-pyrimidin-4-ylamino]-benzenesulfonamide,
Compound 367: 3-[6-(2-Methoxy-phenyl)-pyrimidin-4-ylamino]-N-propyl-benzenesulfonamide,
Compound 368: [6-(2-Methoxy-phenyl)-pyrimidin-4-yl]-(2-methyl-1H-indol-5-yl)-amine,
Compound 369: N-(2-Methoxyethyl)-3-[6-(2-methoxy-phenyl)-pyrimidin-4-ylamino]-benzenesulfonamide,
Compound 370: N-tert-Butyl-3-[6-(2-methoxy-phenyl)-pyrimidin-4-ylamino]-benzenesulfonamide,
Compound 371: [6-(2-Methoxy-phenyl)-pyrimidin-4-yl]-pyridin-2-ylmethyl-amine,
Compound 372: [6-(2-Methoxy-phenyl)pyrimidin-4-yl]-pyridin-3-ylmethyl-amine,
Compound 373: [6-(2-Methoxy-phenyl)-pyrimidin-4-yl]-pyridin-4-ylmethyl-amine,
Compound 374: 5-[6-(2-Methoxy-phenyl)-pyrimidin-4-ylamino]-2-methyl-benzenesulfonamide,
Compound 375: N-(2-Methoxy-ethyl)-5-[6-(2-methoxy-phenyl)-pyrimidin-4-ylamino]-2-methyl-benzenesulfonamide,
Compound 376: N-(2-Hydroxy-ethyl)-5-[6-(2-methoxy-phenyl)-pyrimidin-4-ylamino]-2-methyl-benzenesulfonamide,
Compound 377: N,N-Diethyl-N′-[6-(2-methoxy-phenyl)-pyrimidin-yl]-benzene-1,4-diamine,
Compound 378: 1-(4-Chloro-3-trifluoromethyl-phenyl)-3-{5-[6-(2-methoxy-phenyl)pyrimidin-4-ylamino]-2-methyl-phenyl}-urea,
Compound 379: 1-Cyclohexyl-3-{5-[6-(2-methoxy-phenyl)-pyrimidin-4-ylamino]-2-methyl-phenyl}-urea,
Compound 380: [6-(2-Methoxy-phenyl)pyrimidin-4-yl]-(4-pyrrolidin-1-yl-phenyl)amine,
Compound 381: 4-Chloro-N-1-[6-(2-methoxy-phenyl)pyrimidin-4-yl]-benzene-1,3-diamine,
Compound 382: 1-Isopropyl-3-{5-[6-(2-methoxy-phenyl)pyrimidin-4-yamino]-2-methyl-phenyl}-urea,
Compound 383: 1-{5-[6-(2-Methoxy-phenyl)-pyrimidin-4-ylamino]-2-methyl-phenyl}-3-(2-morpholin-4-yl-ethyl)-urea,
Compound 384: 1-(2-Dimethylamino-ethyl)-3-{5-[6-(2-methoxy-phenyl)-pyrimidin-4-ylamino]-2-methyl-phenyl}-urea,
Compound 385: (4-Chloro-3-nitro-phenyl)-[6-(2-methoxy-phenyl)-pyrimidin-4-yl]-amine.

The activity of 4,6-disubstituted aminopyrimidine derivatives as described in WO 2005/026129 in the treatment of inflammatory and/or neuropathic pain is evidenced herein inter alia in experimental models of inflammatory and/or neuropathic pain in mice.

### Pharmaceutical Compositions

The present invention is also directed to pharmaceutical compositions comprising at least one cyclin-dependent kinase inhibitor as an active ingredient together with at least one pharmaceutically acceptable (i.e. non-toxic) carrier, excipient and/or diluent for administration to a subject in need thereof.

In a preferred embodiment, said CDK inhibitor inhibits CDK9.

In a particular preferred embodiment, said CDK inhibitor is a selective inhibitor of CDK9.

Furthermore, the invention also comprises compositions combining at least two inhibitors of CDK and/or pharmaceutically acceptable salts thereof. Said at least two inhibitors may inhibit the same cyclin-dependent kinase or may also inhibit different types of cylin-dependent kinases, e.g. one inhibitor in the composition may inhibit CDK9 while the other inhibitor is capable of inhibiting CDK2, for example.

In a further preferred embodiment, the invention is directed to compositions comprising at least one cyclin-dependent kinase inhibitor in combination with one or more additional pain-reducing agents and to a method of administering such a composition. Specifically, said at least one cyclin-dependent kinase inhibitor inhibits CDK9.

An individual pain medication often provides only partially effective pain alleviation because it interferes with just one pain-transducing pathway out of many. Thus, it is also intended to administer CDK inhibitors in combination with a pain-reducing (analgesic) agent that acts at a different point in the pain perception process.

An “analgesic agent” comprises a molecule or combination of molecules that causes a reduction in pain. An analgesic agent employs a mechanism of action other than inhibition of CDK.

One class of analgesics, such as nonsteroidal anti-inflammatory drugs (NSAIDs), down-regulates the chemical messengers of the stimuli that are detected by the nociceptors and another class of drugs, such as opioids, alters the processing of nociceptive information in the CNS. Other analgesics are local anesthetics, anticonvulsants and antidepressants such as tricyclic antidepressants. Administering one or more classes of drug in addition to CDK inhibitors can provide even more effective amelioration of pain.

Preferred NSAIDs for use in the methods and compositions of the present invention are aspirin, acetaminophen, ibuprofen, and indomethacin. Furthermore, cyclooxygenase-2 (COX-2) inhibitors, such as specific COX-2 inhibitors (e.g. celecoxib, COX189, and rofecoxib) may also be used as an analgesic agent in the methods or compositions of the present invention.

Preferred tricyclic antidepressants are selected from the group consisting of Clomipramine, Amoxapine, Nortriptyline, Amitriptyline, Imipramine, Desipramine, Doxepin, Trimipramine, Protriptylin, and Imipramine pamoate.

Furthermore, the use of anticonvulsants (e.g. gabapentin), GABAB agonists (e.g. L-baclofen), opioids, vanniloid receptor antagonists and cannabinoid (CB) receptor agonists, e.g. CB1 receptor agonists as analgesic is also preferred in the methods and compositions in the present invention.

In preparing cyclin-dependent kinase inhibitor compositions of this invention, one can follow the standard recommendations of well-known pharmaceutical sources such as Remington: The Science and Practice of Pharmacy, 19^{th} ed. (Mack Publishing, 1995).

The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutaneous, rectal, subcutaneous, sublingual, topical, or transdermal application, wherein said preparations in addition to typical vehicles and/or diluents contain at least one CDK inhibitor and/or a pharmaceutically acceptable salt thereof as active ingredient.

The pharmaceutical compositions according to the present invention containing at least one CDK inhibitor and/or a pharmaceutically acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like.

Moreover, suitable binders, lubricants, disintegrating agents and coloring agents may also be incorporated into the tablet or capsule. Powders and tablets may contain about 5 to about 95% by weight of a cyclin-dependent kinase inhibitor or the respective pharmaceutically active salt as active ingredient.

Suitable binders include starch, gelatin, natural sugars, corn sweeteners, natural and synthetic gums such as acacia, sodium alginate, carboxymethylcellulose, polyethylene glycol and waxes. Suitable lubricants include boric acid, sodium benzoate, sodium acetate, sodium chloride, and the like.

Suitable disintegrants include starch, methylcellulose, guar gum, and the like.

Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect(s), e.g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g. nitrogen.

For preparing suppositories, a low melting wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified.

Also included are solid form preparations, which are intended to be converted, shortly before use, to liquid form preparations for either oral or parenteral administration. Such liquid forms include solutions, suspensions, and emulsions.

CDK inhibitors may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e.g. of methylcellulose, polyvinyl alcohols, or denatured gelatins or starch for holding or containing compositions comprising the active ingredient(s). Capsules with hard shells are typically made of blended or relatively high gel strength gelatins from bones or pork skin. The capsule itself may contain small amounts of dyes, opaquing agents, plasticisers and/or preservatives.

“Tablet” refers to a compressed or moulded solid dosage form which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powder for constitution refers to powder blends containing the active ingredients and suitable diluents which can be suspended e.g. in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75% by weight, and more preferably from about 30 to about 60% by weight.

The term disintegrants refers to materials added to the composition to support disintegration and release of the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodiumcroscaramellose, alginates such as alginic acid and sodium alginate, clays such as bentonites, and effervescent mixtures. The amount of disintegrant in the composition may range from about 2 to about 20% by weight of the composition, more preferably from about 5 to about 10% by weight.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20% by weight of the composition, preferably from about 3 to about 10% by weight, and more preferably from about 3 to about 6% by weight.

Lubricants refer to a class of substances which are added to the dosage form to enable the tablet, granules etc. after being compressed to release from the mould by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5% by weight of the composition, preferably from about 0.5 to about 2% by weight, and more preferably from about 0.3 to about 1.5% by weight of the composition.

Glidents are materials that prevent baking of the components of the pharmaceutical composition together and improve the flow characteristics of granulate so that flow is smooth and uniform. Suitable glidents include silicon dioxide and talc.

The amount of glident in the composition may range from about 0.1 to about 5% by weight of the final composition, preferably from about 0.5 to about 2% by weight.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5% by weight of the composition, preferably from about 0.1 to about 1% by weight.

The present invention relates to the administration of compositions containing as active ingredient a cyclin-dependent kinase inhibitor to a subject in need thereof for the treatment of any type of pain.

“A subject in need thereof” comprises an animal, preferably a mammal, and most preferably a human, expected to experience pain in the near future or which has ongoing pain experience. Such animal or human may have an ongoing condition that is causing pain currently and is likely to continue to cause pain, or the animal or human has been, is or will be enduring a procedure or event that usually has painful consequences. Chronic painful conditions such as diabetic neuropathic hyperalgesia and collagen vascular diseases are examples of the first type; dental work, particularly in an area of inflammation or nerve damage, and toxin exposure (including exposure to chemotherapeutic agents) are examples of the latter type.

In order to achieve the desired hypoalgesic effect, the respective cyclin-dependent kinase inhibitor has to be administered in a therapeutically effective amount.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated. In the context of the present invention, a therapeutically effective amount comprises an amount that reduces/ameliorates/treats any type of pain, in particular inflammatory or neuropathic pain. Specifically, a therapeutically effective amount denotes an amount which exerts a hypoalgesic effect in the subject to be treated.

Such effective amount will vary from subject to subject depending on the subject's normal sensitivity to pain, its height, weight, age, and health, the source of the pain, the mode of administering the inhibitor of CDKs, the particular inhibitor administered, and other factors. As a result, it is advisable to empirically determine an effective amount for a particular subject under a particular set of circumstances.

The invention is further illustrated by the following non-limiting examples.

### EXAMPLES

### Behavioral Animal Models for the Analysis of Inflammatory and Neuropathic Pain

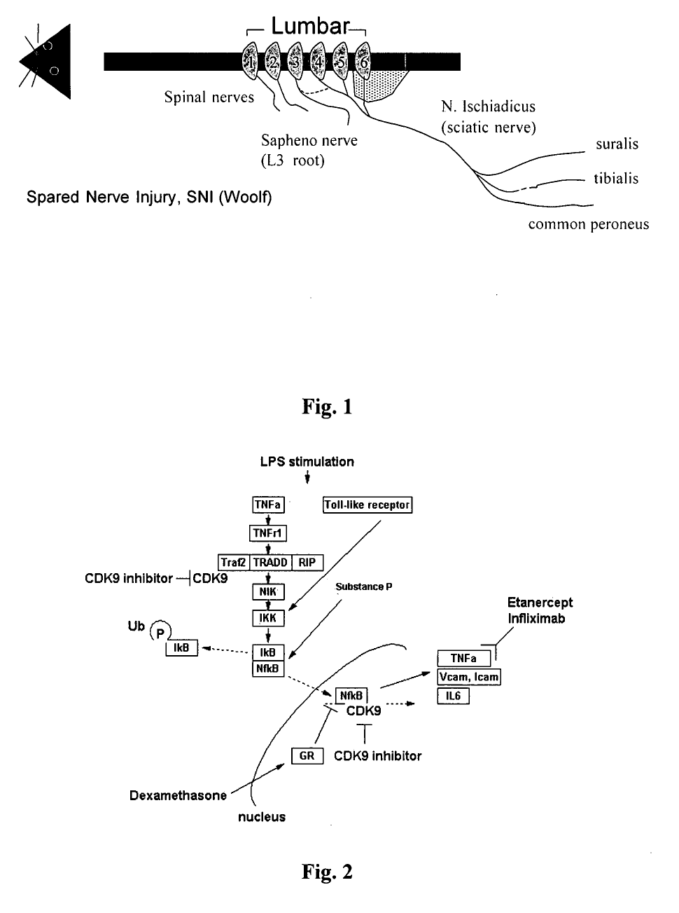

Several animal models for the analysis of inflammatory and neuropathic pain are known. Said models share the common feature that after e.g., induction of a nerve lesion (e.g., spared nerve injury, SNI) or after exposing experimental animals to a noxious stimulus (e.g., injection of formalin or carrageenan), the signs of pain as induced by said interventions are measured by quantifiable behavioral components such as, e.g., paw withdrawal threshold to mechanical stimulation with von Frey hairs (or to thermal stimulation using a laser source or licking behaviour). These reactions are interpreted as being equivalent to mechanical and thermal allodynia (hypersensitivity to mechanical stimuli) or hyperalgesia in humans. The spared nerve injury model (SNI model, as developed by Decosterd and Woolf (2000), see FIG. 1) is characterized by the induction of clinically relevant nerve lesions and after surgical intervention, subsequent behavioral experiments (e.g., von Frey Assay). Said model constitutes a common nerve injury model which consists of ligation and section of two branches of the sciatic nerve (namely tibial and common peroneal nerves) leaving the sural nerve intact. The SNI model results in early (less than 24 hours), prolonged and substantial changes in mechanical and cold sensitivity that closely mimic the features of clinical neuropathic pain. Animals with these types of nerve injury have been shown to develop abnormal pain sensations and hypersensitivity to mechanical stimuli (allodynia) similar to those reported by neuropathic pain patients.

Alternatively, the formalin assay in mice is a valid and reliable behavioral model of nociception in inflammatory and neuropathic pain. It is sensitive to various classes of analgesic drugs (Hunskaar S & Hole K, Pain 1987) The noxious stimulus consists of an injection of 10 μl diluted formalin (2% in saline) under the skin of the dorsal surface of the left hindpaw (subcutaneous or interplantar into the left hindpaw). The response is licking and flinching of the injected paw.

An additional assay, the so-called carrageenan assay, comprises a subcutaneous injection of 25 μl of 1% carrageenan (in saline) into a single hind paw (ipsi-lateral paw) of mice. Subsequent inflammation results in long lasting swelling and hypersensitivity (against mechanical and thermal stimuli) of the paw. The carrageenan assay is a standard laboratory assay used to predict anti-inflammatory activity of test compounds. Paw edema measurements and Hargreaves Assay (which measures withdrawal of paws due to thermal stimulation via a light source) are used for read out.

In the present invention, the effect of administration of cyclin-dependent kinase (CDK)-inhibiting compounds on the development of inflammatory and neuropathic pain was assayed in an SNI model, in a carrageenan and in a formalin assay. The experimental procedure and results are described in detail below.

### Example 1

### A. Spared Nerve Injury (SNI)— Model of Chronic Neuropathic Pain

As outlined above, the spared nerve injury (SNI) model (see FIG. 1) involves a lesion of two of the three terminal branches of the sciatic nerve (tibial and common peroneal nerves) of experimental animals, leaving the sural nerve intact. SNI results in mechanical and thermal allodynia in the non-injured sural nerve skin territory (Decosterd & Woolf, Pain 2000; 87:149-158; Tsujino et al., Mol. Cel. Neurosci. 2000; 15:170-182).

### 1. Induction of Spared Nerve Injury (Nerve Lesion) in Wildtype Mice

Wildtype mice (strain C3HeB/FeJ) (age, sex and weight matched) were anesthetized with Hypnorm (0.315 mg/ml fentanyl citrate+10 mg/ml fluanisone; Janssen)/Hypnovel (5 mg/ml midazolam; Roche Applied Sciences)/water at a ratio of 1:1:2 at 4 μl/g prior to surgical preparation.

Subsequently, an incision was made under aseptic precautions in the ipsi-lateral right hind leg of all mice just above the level of the knee, exposing the three terminal branches of the sciatic nerve: the common peroneal, tibial, and sural nerves. The common peroneal and tibial nerves were ligated tightly with 7/0 silk and sectioned distal to the ligation removing ≈2 mm of distal nerve stump. The sural branch remained untouched during the procedure (denoted herein “SNI ipsi”). The overlying muscle and skin was sutured, and the animals were allowed to recover and to permit wound healing. In the same mice, the sciatic nerve branches of the contra-lateral left hind leg were exposed but not lesioned (denoted herein “SNI contra-lateral”). Mice that underwent spared nerve injury are hereinafter denoted “SNI mice”.

### 2. Administration of CDK-Inhibiting Compounds to SNI Mice

After recovery from surgery and wound healing, SNI mice received intraperitoneal (i.p.) injections of CDK-inhibiting compounds. In particular, compound A and B as disclosed herein, belonging to the class of 4,6-disubstituted aminopyrimidine derivatives as described in WO2005/026129 were administered.

30 mg/kg of a CDK inhibitor, dissolved in DMA/Labrafil (10:90) was administered by a single i.p. injection 30 min prior to von Frey measurements (mechanical allodynia). As a negative control, the same amount (200 μl) of DMA/Labrafil (10:90) vehicle was administered by a single i.p. injection 30 min prior to von Frey measurements.

Injection of inhibitor or vehicle, and subsequent measurements of paw withdrawal threshold upon mechanical stimulation in von Frey assays were performed at day 0, day 7, day 21 and day 28 post SNI. Reflex nociceptive responses to mechanical stimulation were measured in a von Frey assay 30 min after each injection. An additional von Frey measurement was performed at day 14, but without prior injection of compound or vehicle.

The effect of administration of CDK inhibitors to SNI mice on the development of mechanical allodynia was analyzed in a von Frey assay, as described below.

3. Behavioral Testing of SNI Mice after Administration of CDK-Inhibiting Compounds (Von Frey Assay)

Mice which underwent SNI and subsequent administration of the CDK-inhibiting compounds of the present invention were tested for signs of mechanical allodynia post nerve injury and post administration in a von Frey assay (Decosterd and Woolf, Pain 2000; 87:149-158). This assay determines the mechanical threshold upon which a stimulus, which normally is not painful, is recognized by an animal as uncomfortable or painful. SNI ipsi and SNI contra baselines, respectively, were established.

Mechanical thresholds of SNI mice were quantified using the up-down method based on Chaplan et al. (1994) and Malmberg and Basbaum (1998):

Mice were placed in plexiglass cylinders of about 9.5 cm in diameter, 14 cm high with four vent holes towards the top and a Plexiglas lid. The cylinders were placed on an elevated mesh surface (7×7 mm squares). Prior to the day of testing, the mice were adapted to the testing cylinders for 1-2 hours. On the day of testing the mice were adapted to the cylinders for about an hour, wherein the adaptation time depends on factors such as the strain of the mouse and the number of times they have been tested previously. In general, testing may begin once the mice are calm and stop exploring the new environment.

For testing mice, filaments 2.44, 2.83, 3.22, 3.61, 3.84, 4.08, and 4.31 (force range=0.04 to 2.0 g) were used. The 3.61 mN filament was applied first. Said filament was gently applied to the plantar surface of one paw, allowed to bend, and held in position for 2-4 seconds. Whenever a positive response to the stimulus (flexion reaction) occurred, the next weaker von Frey filament was applied; whenever a negative response (no reaction) occurred, the next stronger force was applied. The test was continued until the response to 4 more stimuli after the first change in response had been obtained. The highest force tested was 4.31. The cut-off threshold was 2 g.

The series of scores (i.e., “flexion reaction” and “no reaction”) and the force of the last filament applied were used to determine the mechanical threshold as described in Chaplan et al., Journal of Neuroscience Methods, 53(1):55-63, 1994. The threshold determined is that to which the animal would be expected to respond to 50% of the time. Mice were sacrificed after von Frey measurements were accomplished.

### 4. CDK Inhibitor Compound A has a Hypoalgesic Effect in SNI Mice

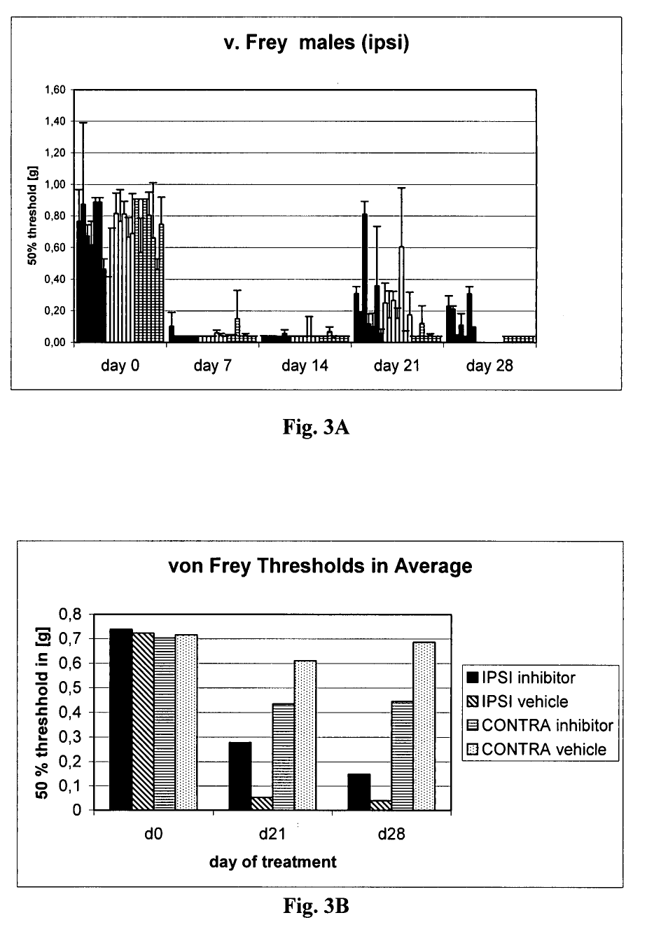

Compound A as described herein was administered to SNI mice as described above. Von Frey measurements were performed as described above. It could be shown that compound A had a hypoalgesic effect on SNI mice. The results of administration of compound A to SNI mice are shown in FIGS. 3A, 3B.

FIG. 3A depicts the results of von Frey measurements performed with SNI mice. Von Frey measurements were performed at ipsi-lateral and contra-lateral paws of the animals at 4 different time points (day 0, day 7, day 14, day 21 and day 28 after surgery). Day 0 represents baseline behavior prior to SNI surgery. The “day 7” group (black) received treatment (30 mg/kg compound A i.p. or vehicle) at day 7, 21 and 28, the “day 21” group (white) received the same treatment at day 21 only while the “vehicle” group (striped) received vehicle only (10% DMA; 90% Labrafil) at day 7, 21 and day 28.

FIG. 3A displays the individual threshold of each animal. At day 7, treatment with compound A (“black” group) does not show any effect in the von Frey assay. All three groups display high sensitivity (=low threshold) against the mechanical stimulus. Low thresholds were also seen at day 14 without any prior treatment. At day 21, however, treatment with 30 mg/kg compound A i.p., 30 minutes prior to measurements did show an effect. While the (striped) vehicle control group remained sensitive with low thresholds similar to measurements on day 7 and 14, animals treated with compound A (black and white group) displayed a significant increase of threshold values, indicating reduced sensitivity to mechanical stimuli (reduced allodynia). At day 28, the “day 7” group (black) was treated again at midnight and 8.00 a.m. with 30 mg/kg compound A each and thresholds were measured at 8.30 a.m., whereas the vehicle control group (striped) received solvent at the same time. Again, significant increases in thresholds of animals treated with compound A were observed. In comparison, animals treated with vehicle alone displayed low thresholds and high allodynia.

The average values of von Frey measurements are shown in FIG. 3B, depicting the results of von Frey measurements performed in individual SNI mice as described above (FIG. 3A). In FIG. 3B, the data shown in FIG. 3A are displayed in average values for compound A and vehicle treated ipsi-lateral and contra-lateral paws of animals treated at day 21 and 28 (white group=compound; striped group=vehicle). The threshold value of the ipsi-lateral paw of vehicle-treated mice shows that these animals are very sensitive (<0.1 g) at day 21 and 28, whereas the ipsi-lateral paws of compound-treated mice are less sensitive (˜0.3 g). The contra-lateral paw of vehicle-treated mice is similar to baseline sensitivity at day 0 (˜0.7 g) whereas compound-treated mice show a reduced threshold at day 21 and 28 (˜0.4 g). These findings indicate that compound A is effective as a hypoalgesic drug in models of chronic neuropathic pain.

### 4. CDK Inhibitor Compound B has a Hypoalgesic Effect in SNI Mice

Compound B as described herein was administered to SNI mice as described above. Von Frey measurements were performed as described above. It could be shown that compound B had a hypoalgesic effect on SNI mice. The results of administration of compound B to SNI mice are shown in FIG. 9, demonstrating that intraperitoneal treatment with 30 mg/kg compound B 30 minutes before von Frey testing resulted in a significant reduction of SNI-induced allodynia.

### Example 2

### A. Formalin Assay—Model of Inflammatory/Chronic Neuropathic Pain

The formalin assay in mice is a valid and reliable behavioral model of nociception and is sensitive to various classes of analgesic drugs (Hunskaar S & Hole K, Pain. 1987, 30(1):103-14.) The noxious stimulus consists of a subcutaneous or an intraplantar injection of 10 μl diluted formalin (2% in saline) into the left hind paw. The response is licking and flinching of the injected paw. The response shows two phases, which reflect different parts of the inflammatory process (Abbott et al., Pain 1995), an early/acute phase 0-5 min post-injection, and a late/chronic phase 5-30 min post-injection.

### 1. Injection of Formalin and Administration of CDK-Inhibiting Compound

Age, sex and weight matched wildtype mice (C3HeB/FeJ) were used in this assay. Prior to formalin injection, the animals were randomly subdivided into experimental groups of 10 animals each. Thirty minutes prior to formalin injection, CDK inhibitor (30 mg/kg of compound B or compound A, dissolved in DMA/Labrafil (10:90)) was administered by i.p. injection. Similarly, Iκ Kinase (IKK) inhibitor (30 mg/kg) in DMA/Labrafil (positive control), or vehicle alone (DMA/Labrafil, 10:90) (negative control) were administered by i.p. injection 30 min before formalin injection.

For formalin injection, the mouse was held with a paper towel in order to avoid disturbance of the injection by movements. The injected hind paw was held between thumb and forefinger and 10 μl of Formalin (2%) were injected subcutaneously (s.c.) between the two front tori into the plantar hind paw using a Hamilton syringe. The behavior of the formalin- and CDK-inhibitor-treated mice was analyzed as described below.

2. Behavioral Analysis of Mice after Injection of Formalin and after Administration of CDK-Inhibiting Compound

The behaviour of the formalin-treated mice, i.e. licking and flinching, was monitored by an automated tracking system (Ethovision 3.0 Color Pro, Noldus, Wageningen, Netherlands) over a defined period of time: measurement was initiated 5 min after formalin injection and terminated 30 min after formalin injection. This time frame covers phase II of formalin-induced nociception (pain), which is hyperalgesia.

Two different fluorescent dyes were used for topically marking the injected hind paw (yellow dye) (Lumogenyellow; BASF Pigment, Cologne, Germany) and the contralateral paw (blue dye) (Lumogenviolet; Kremer Pigmente, Aichstetten, Germany), respectively. To determine licking behaviour, mice were monitored with a CCD camera. After monitoring and recording, the video was analyzed using the EthoVision software (Ethovision 3.0 Color Pro, Noldus, Wageningen, Netherlands) or by manual analysis. Fluorescent dot sizes and fluorescence intensities were measured and reduction of fluorescent dot size through licking and biting was calculated. The overall licking time intensity was automatically calculated by comparison of dot size reduction of treated versus untreated paws.

As another possibility of assay read-out, the licking behaviour of the individual animals was tracked manually based on video files. Licking times were recorded over 30 minutes after formalin injection and subdivided for three different licking zones (dorsum, plantar, toes). Overall licking times can be calculated for each animal as well as each experimental group and can be used as a parameter for determination of compound efficacy.

As a result it was found that mice receiving vehicle treatment prior to formalin injection (negative control) displayed a prolonged licking time and a significant reduction of fluorescent dot size at the formalin-treated paw, thus indicating a high sensitivity of the mice to the noxious stimulus.

In contrast, a reduction in licking time and consequently, no significant reduction of fluorescent dot size of the formalin-treated paw was observed in mice treated with compound B/formalin or compound A/formalin, demonstrating reduced sensitivity of said mice to the noxious stimulus. The same effect, i.e. a reduction in licking time and a minor change in fluorescent dot size, was observed in control mice treated with Iκ kinase inhibitor (IKK; for function of IKK see FIG. 2, positive control).

This finding is indicative of reduced inflammatory/chronic inflammatory pain perception in CDK9 inhibitor-treated mice and for a hypoalgesic effect of CDK inhibitors compound B and compound A.

### 3. Administration of Compound B has a Hypoalgesic Effect Informalin-Treated Mice

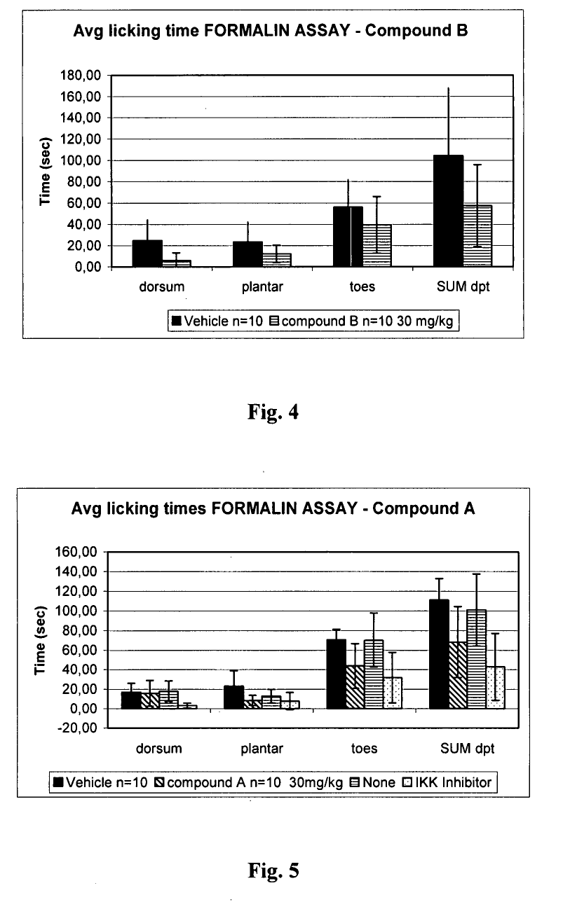

The results of administration of compound B to formalin-treated mice are shown in FIG. 4.

FIG. 4 displays the averages of licking time observed in mice (n=10) treated with compound B (30 mg/kg) or vehicle (manual analysis). Total licking time is significantly reduced in mice treated with compound B (FIG. 4, compound B), comprising all three parts of the paw observed: dorsal side (FIG. 4, dorsum), plantar side (FIG. 4, plantar) and toes (FIG. 4, toes) of injected paws, compared to the vehicle control (FIG. 4, Vehicle). “SUM dpt” displays the sum of dorsum, plantar and toe licking times.

This observation is indicative for reduced inflammatory/chronic inflammatory pain perception in CDK9 inhibitor-treated mice and for a hypoalgesic effect of CDK9 inhibitor compound B.

### 4. Administration of Compound A has a Hypoalgesic Effect in Formalin-Treated Mice

The results of administration of compound A to formalin-treated mice are shown in FIG. 5.

FIG. 5 displays the averages of licking time observed in mice (n=10) treated with compound A (30 mg/kg), IKK inhibitor (30 mg/kg, positive control), vehicle (DMA/Labrafil, negative control), or no treatment (none), respectively, all analyzed by manual observation. Licking time is clearly reduced in mice treated with compound A (FIG. 5, compound A) as well as in IKK inhibitor-treated mice (FIG. 5, IKK inhibitor) compared to the “Vehicle” and “None” controls (FIG. 5, Vehicle and None). “SUM dpt” displays the sum of dorsum, plantar and toe licking times.

This observation is indicative for reduced inflammatory/chronic inflammatory pain perception in CDK9 inhibitor-treated mice and for a hypoalgesic effect of CDK9 inhibitor compound A.

### Example 3

### A. Carrageenan Assay—Model of Inflammatory Pain

The model of carrageenan-induced paw edema constitutes a standard laboratory assay used to predict anti-inflammatory activity of therapeutically active compounds and reduction of inflammation-induced pain perception achieved by administration of therapeutically active compounds.

The basic measurement constitutes in the measurement of edema size and of mechanical as well as thermal hypersensitivity in response to irritants, such as carrageenan.

Inflammation and subsequent inflammatory pain is induced by subcutaneous injection of 25 μl of 1% carrageenan (in saline) into the hind paw (ipsi-lateral paw) of mice. Groups of 10 mice each received compound A, 30 mg/kg body weight, vehicle (DMA/Labrafil; 10:90) and saline (physiol. NaCl) by i.p. injection 30 min prior to carrageenan injection. Contra-lateral paws did not receive carrageenan injection.

### 1. Effects of Administration of Compound A on Carrageenan-Treated Mice

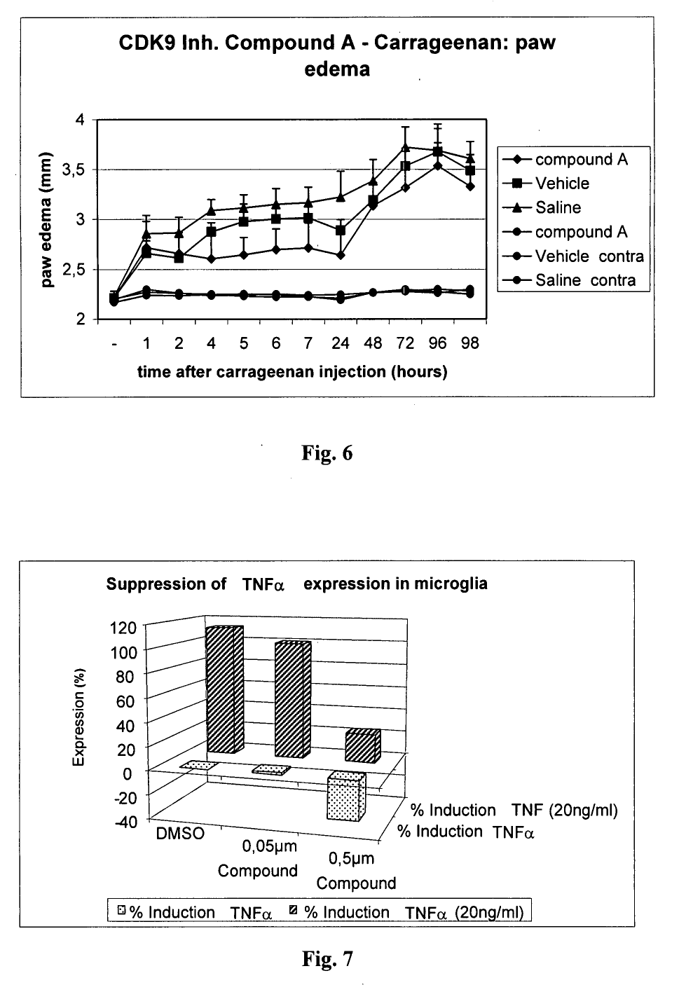

The effects of administration of compound A to carrageenan-treated mice are shown in FIG. 6.

Paw edema induced by carrageenan injection was detected by increased paw size measured from dorsal to plantar at the metatarsus region of the injected (ipsi-lateral) paws. Sizes of ipsi- and contra-lateral paws (see FIG. 6, paw size [mm]) served as surrogate markers for inflammation and were measured at several time points after carrageenan injection: 1 h before injection (−1), immediately after injection, 1 h (1), 2 h (2), 4 h (4), 5 h (5), 6 h (6), 7 h (7), 24 h (24), 48 h (48), 72 h (72), 96 h (96), and 98 h (98) after injection.

As depicted in FIG. 6, the paw size of all mice increased by 2 to 3 mm (+10%) within the first hour after carrageenan injection, irrespective of the type of injection 30 minutes prior to carrageenan. During the time course, mice which received compound A treatment prior to carrageenan injection displayed a reduction of the edema until 24 h after carrageenan injection: the increase in paw size dropped from 10% down to 8%. In contrast, the paw size of the control mice increased by 30% in average at this time point. After 24 h post carrageenan injection, the size of all paws treated with carrageenan increased to reach their maximum at 96 h after injection.

FIG. 6 depicts the course of developing hind leg paw edema over a time course of 98 h after carrageenan injection into mice hind paws, and respective controls thereof. Paw edema were detected and measured by an increase of paw size (in mm) from dorsal to plantar at the metatarsus region of a paw (FIG. 6, paw size (mm)). I.p. injection of compound A (FIG. 6, compound A), vehicle (FIG. 6, vehicle) and saline (FIG. 6, Saline) as negative controls was performed 30 min before intraplantar carrageenan injection. Sizes of ipsi-lateral and contra-lateral paws were determined at several time points during the time course (FIG. 6, hours after injection)].

### 2. Effects of Administration of Compound B on Carrageenan-Treated Mice as Measured by Hargreaves Assay

As a read-out of the carrageenan assay, a Hargreaves Assay may be performed, wherein said assay allows the measuring of thermal sensitivity to radiant heat. The Hargreaves assay (Hargreaves et al., 1988) measures nociceptive sensitivity in a freely moving animal by focusing a radiant heat source on the plantar surface of an animal's hindpaw as it stands in a plexiglass chamber. Specifically, the lower side of a paw is exposed to a luminous source, generating a temperature of, e.g. 55° C. Thermal sensitivity is measured as latency between start of exposure and lifting/pulling the exposed paw.

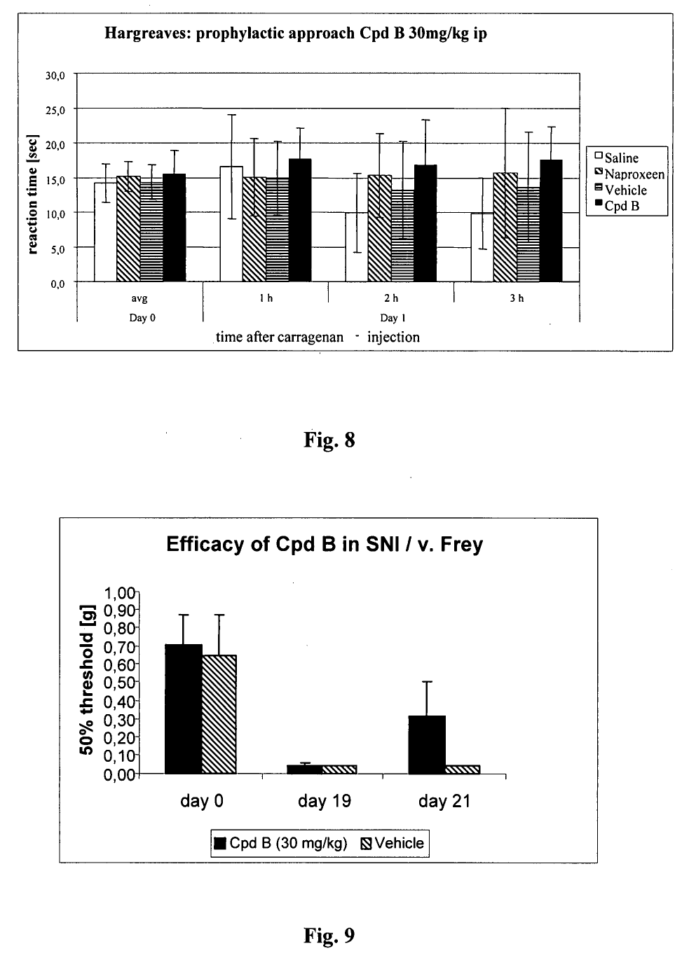

The results of a Hargreaves assay performed after administration of a CDK inhibitor are shown in FIG. 8.

Mice treated with 30 mg/kg of compound B and carrageenan, or with Naproxen and carrageenan, or with solvent and carrageenan, respectively, were subjected to a Hargreaves assay. Compound B was administered as an i.p. injection 30 min prior to administration of carrageenan. Mice treated with compound B displayed a longer latency, thus indicating that intraperitoneal treatment with 30 mg/kg compound B 30 minutes before carrageenan application results in significant reduction of thermal hyperalgesia detected by the Hargreaves assay comparable to 50 mg/kg Naproxen.

### Example 4

In order to validate the hypothesis that TNFα stimulation is regulated by an autocrine loop (i.e., TNFα induces expression of TNFα; see FIG. 2), a microglial cell line (EOC20) of human origin was stimulated in vitro by addition of TNFα (20 ng/ml). Microglial cells which did not undergo TNFα stimulation were used as controls.

1 hour prior to addition of TNFα, stimulated or unstimulated cells, respectively, were treated with DMSO (vehicle control), or 0.5 μM and 0.05 μM, respectively, of compound A. Cells were harvested 48 h after addition of TNFα. RNA was isolated from the samples and the amount of amplicon of TNFα (and of other cytokines) and of several housekeeping genes was measured by light cycler-based Real-Time PCR.

Samples from TNFα-stimulated cells showed a clear increase of TNFα amplicon of around +100% (normalized with regard to housekeeping genes and in comparison to the negative control=no TNFα+DMSO). Addition of compound A effected a dose-dependent reduction of said inductive effect. A reduction of TNFα message was even detected in unstimulated cells that had been treated with compound A. These findings show that compound A exerts a suppressive effect on expression of TNFα.

### Example 5

### In Vitro Kinase Inhibition Assays

IC50 profiles of compounds according to the present invention for cyclin-dependent kinases such as CDK2/CycA, CDK4/CycD1, CDK6/CycD1 and CDK9/CycT can be measured in in vitro enzymatic kinase inhibition assays. IC50 values as obtained in these assays may be used for evaluating the specific selectivity and potency of the compounds with respect to CDK9 inhibition.

Results obtained in these assays may be used to select compounds displaying specificity for CDK9. Specifically, it is intended to distinguish the CDK9-specific compounds from other compounds having significant inhibitory potency also with regard to other CDKs, i.e. on some or all of CDKs 2, 4 and 6.

In vitro kinase inhibition assays may be performed according to the following protocol:

1. Test compounds

Compounds are used as 1×10⁻⁰² M stock solutions in 100% DMSO, 100 μl each in column 2 of three 96-well V-shaped microtiter plates (in the following, said plates are referred to as “master plates”).

Subsequently, the 1×10⁻⁰² M stock solutions in column 2 of the master plates are subjected to a serial, semi-logarithmic dilution using 100% DMSO as a solvent, resulting in 10 different concentrations, the dilution endpoint being 3×10⁻⁰⁷ M/100% DMSO in column 12. Column 1 and 7 are filled with 100% DMSO as controls. Subsequently, 2×5 μl of each well of the serial diluted copy plates are aliquoted in 2 identical sets of “compound dilution plates”, using a 96-channel pipettor.

On the day of the kinase inhibition assay, 45 μl H₂O are added to each well of a set of compound dilution plates. To minimize precipitation, H₂O is added to the plates only a few minutes before the transfer of the compound solutions into the assay plates. The plates are shaken thoroughly, resulting in “compound dilution plates/10% DMSO” with a concentration of 1×10⁻⁰³ M/10% DMSO to 3×10⁻⁰⁸ M/10% DMSO in semilog steps. These plates are used for the transfer of 5 μl compound solution into the “assay plates”. The compound dilution plates are discarded at the end of the working day. For the assays (see below), 5 μl solution from each well of the compound dilution plates are transferred into the assay plates. The final volume of the assay is 50 μl. All compounds are tested at 10 final assay concentrations in the range from 1×10⁻⁰⁴ M to 3×10⁻⁰⁹ M. The final DMSO concentration in the reaction mixtures is 1% in all cases.

### 2. Recombinant Protein Kinases

For the determination of inhibitory profiles, the following protein kinases were used: CDK2/CycA, CDK4/CycD1, CDK6/CycD1 and CDK9/CycT. Protein kinases CDK1/CycB, CDK3/CycE, CDK5/p35NCK and CDK7/CycH/MAT1 may be included in the assay as well. Said protein kinases are expressed in Sf9 insect cells as human recombinant GST-fusion proteins or His-tagged proteins by means of the baculovirus expression system. Kinases are purified by affinity chromatography using either GSH-agarose (Sigma) or Ni-NTH-agarose (Qiagen). The purity of each kinase is determined by SDS-PAGE/silver staining and the identity of each kinase is verified by western blot analysis with kinase specific antibodies or by mass spectroscopy.

### 3. Protein Kinase Assay

All kinase assays are performed in 96-well FlashPlates™ from Perkin Elmer/NEN (Boston, Mass., USA) in a 50 μl reaction volume. The reaction mixture is pipetted in four steps in the following order:
20 μl of assay buffer (standard buffer)
5 μl of ATP solution (in H₂O)
5 μl of test compound (in 10% DMSO)
10 μl of substrate/10 μl of enzyme solution (premixed)

The assay for all enzymes contains 60 mM HEPES-NaOH, pH 7.5, 3 mM MgCl₂, 3 mM MnCl₂, 3 μM Na-Orthovanadate, 1.2 mM DTT, 50 μg/ml PEG20000, 1 μM [γ-³³P]-ATP (approx. 5×10⁰⁵ cpm per well).

The following amounts of enzyme and substrate are used per well:

| | | Kinase | Kinase | | Substrate |
|---|---|---|---|---|---|
| # | Kinase | Lot # | ng/50 μl | Substrate | ng/50 μl |
| 1 | CDK2/CycA | SP005 | 100 | Histone H1 | 250 |
| 2 | CDK4/CycD1 | SP005 | 50 | Rb-CTF (Lot 009) | 500 |
| 3 | CDK6/CycD1 | SP003 | 400 | Rb-CTF (Lot 009) | 500 |
| 4 | CDK9/CycT | 003 | 100 | Rb-CTF (Lot 009) | 1000 |

Reaction mixtures are incubated at 30° C. for 80 minutes. The reaction is stopped with 50 μl of 2% (v/v) H₃PO₄, plates are aspirated and washed two times with 200 μl H₂O or 200 μl 0.9% (w/v) NaCl. Incorporation of ³³P is determined with a microplate scintillation counter (Microbeta, Wallac).

All assays are performed with a BeckmanCoulter/Sagian robotic system.

### 4. Evaluation of Raw Data

The median value of the counts in column 1 (n=8) of each assay plate is defined as “low control”. This value reflects unspecific binding of radioactivity to the plate in the absence of a protein kinase but in the presence of the substrate. The median value of the counts in column 7 of each assay plate (n=8) is taken as the “high control”, i.e. full activity in the absence of any inhibitor. The difference between high and low control is referred to as 100% activity. As part of the data evaluation, the low control value from a particular plate is subtracted from the high control value as well as from all 80 “compound values” of the corresponding plate. The residual activity (in %) for each well of a particular plate is calculated by using the following formula:

Res. Activity (%)=100×[(cpm of compound−low control)/(high control−low control)]

The residual activities for each concentration and the compound IC50 values are calculated using Quattro Workflow V2.0.1.3 (Quattro Research GmbH, Munich, Germany; www.quattro-research.com). The model used is “Sigmoidal response (variable slope)” with parameters “top” fixed at 100% and “bottom” at 0%.

The inhibitory effect of preferred compounds A and B according to the present invention on several CDKs was determined. The obtained IC₅₀ values are shown in Table 1.

TABLE 1
IC50 profiles of compounds A and B in CDK inhibition assays
for kinases CDK1/CycB, CDK2/CycA, CDK3/CycE, CDK4/CycD1;
CDK5/p35NCK, CDK6/CycD1, CDK7/CycH/MAT1 and CDK9/CycT

| | Compound A | Compound B |
|---|---|---|
| Kinase | IC₅₀ (μM) | IC₅₀ (μM) |
| CDK1/CycB | 15.80 | 1.04 |
| CDK2/CycA | 10.10 | 0.58 |
| CDK3/CycE | 18.50 | 0.13 |
| CDK4/CycD1 | 2.79 | 1.42 |
| CDK5/p35NCK | 58.10 | 3.20 |
| CDK6/CycD1 | 1.91 | 0.86 |
| CDK7/CycH/MAT1 | 10.00 | 2.30 |
| CDK9/CycT | 0.63 | 0.08 |

As evident from Table 1, compounds A and B show a high specificity for CDK9. They are thus selective inhibitors of CDK9.

While the invention is described above in connection with preferred or illustrative embodiments, these embodiments are not intended to be exhaustive or limiting of the invention. Rather, the invention is intended to cover all alternatives, modifications and equivalents included within its spirit and scope of the invention, as defined by the appended claims.

### REFERENCES

Abbott, F. V., Franklin, K. B., and Westbrook, R. F. (1995). The formalin test: scoring properties of the first and second phases of the pain response in rats. Pain 60, 91-102
Barboric M. et al., NfκB Binds P-TEFb to Stimulate Transcriptional Elongation by RNA Polymerase II. Molecular Cell, 2001, Vol. 8, 327-337
Besson J. M., The neurobiology of pain. Lancet, 1999, 353(9164), 1610-1615
Brower, New paths to pain relief. Nat Biotechnol, 2000, 18(4), 387-391
Chao S. H. and Price D. H., Flavopiridol inactivates P-TEFb and blocks most RNA polymerase II transcription in vivo. J Biol Chem, 2001, 276(34),31793-9
Chaplan S R, Bach F W, Pogrel J W, Chung J M, and Yaksh, T L. (1994) Quantitative assessment of tactile allodynia in the rat paw. J Neurosci Methods 53: 55-63.
Dai Y. and Grant S., Cyclin-dependent kinase inhibitors. Curr Opin Pharmacol, 2003, 3(4), 362-370
Decosterd and Woolf, Pain 2000; 87:149-158
Falco G. D. et al., CDK9, a member of the cdc2-like family of kinases, binds to gp130, the receptor of the IL-6 family of cytokines. Oncogene, 2002, 21(49), 7464-7470
Filgueira de Azevedo et al., Biochem. and Biophys. Res. Commun. 2002, 293(1), 566-571
Hargreaves, K: Pain 32(1) (1988) 77-88
Hunskaar S, Hole K, Pain. 1987 July; 30(1):103-14.
Huwe et al., Small molecules as inhibitors of cyclin-dependent kinases. Angew Chem Int Ed Engl, 2003, 42(19), 2122-2138
Kim et al., Phosphorylation of the RNA polymerase II carboxyl-terminal domain by CDK9 is directly responsible for human immunodeficiency virus type 1 Tat-activated transcriptional elongation. Mol Cell Biol, 2002, 22(13), 4622-4637.
Koltzenburg M, Neural mechanisms of cutaneous nociceptive pain. Clin J Pain, 2000, 16(3 Suppl), 131-138
Laufer S., Gay S. And Brune K., Inflammation and Rheumatic Diseases—The molecular basis of novel therapies, Thieme, 2003
Lee K. M. et al., Spinal NfκB activation induces COX-2 upregulation and contributes to inflammatory pain hypersensitivity. European Journal of Neuroscience, 2004, Vol. 19, 3375-3381
Liu H. and Herrmann C., Differential Localization and Expression of the CDK9 42 k and 55 k Isoforms. J Cell Physiol, 2004, 203, 251-260
MacLachlan T. K. et al., Binding of CDK9 to TRAF2. J Cell Biochem, 1998, 71(4), 467-478
Malmberg A B and Basbaum A I. (1998) Partial sciatic nerve injury in the mouse as a model of neuropathic pain: behavioral and neuroanatomical correlates. Pain 76: 215-2
Tian B. et al., Identification of direct genomic targets downstream of the NfκB transcription factor mediating TNF signaling. JBC, 2005, as manuscript M500437200
Tsujino et al., Mol. Cel. Neurosci. 2000; 15:170-182
Watkins L. R. et al., Glial proinflammatory cytokines mediate exaggerated pain states: implications for clinical pain. Adv Exp Med. Biol., 2003, 521, 1-21

1. A method of treating pain, wherein said pain comprises inflammatory pain and/or neurophatic neuropathic pain, said method comprising: administering an effective amount of at least one inhibitor of a cyclin-dependent kinase (CDK) to a subject in need thereof.

2. The method according to claim 1, wherein said at least one inhibitor is a selective inhibitor of a CDK.

3-12. (canceled)

13. The method of claim 1, wherein said at least one inhibitor is a selective inhibitor of CDK9.

14. The method of claim 1, wherein said at least one inhibitor is selected from the class of 4,6-disubstituted aminopyrimidine derivatives.

15. The method of claim 1, wherein said at least one inhibitor of a cyclin-dependent kinase is defined by the following general formula (I), stereoisomeric forms thereof, and pharmaceutical acceptable salts thereof: 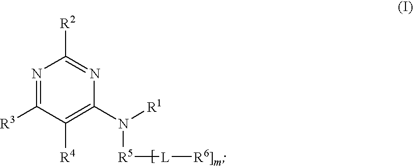
wherein R¹ is selected from the group consisting of: —H, linear C₁-C₆ substituted or unsubstituted alkyl, branched C₁-C₆ substituted or unsubstituted alkyl, linear C₂-C₆ alkenyl, branched C₂-C₆ alkenyl, linear C₂-C₆ alkynyl, and branched C₂-C₆ alkynyl;
R² and R⁴ independently are selected from the group consisting of: —H, linear C₁-C₆ substituted or unsubstituted alkyl, branched C₁-C₆ substituted or unsubstituted alkyl, linear C₂-C₆ alkenyl, branched C₂-C₆ alkenyl, linear C₂-C₆ alkynyl, branched C₂-C₆ alkynyl, aryl, —F, —Cl, —Br, —I, —CN, —NH₂, and —NO₂;
R³ is selected from the group consisting of: —F, —Cl, —Br, —I, substituted or unsubstituted aryl, a substituted or unsubstituted aryl selected from the group consisting of —O-aryl, —NH-aryl, —S-aryl, and —CH═CH-aryl, a substituted or unsubstituted heterocyclyl selected from —O-heterocyclyl, —NH-heterocyclyl, and —S-heterocyclyl, a substituted or unsubstituted heteroaryl, a substituted or unsubstituted heterocyclyl, a substituted or unsubstituted C₃-C₈ cycloalkyl, and —NH—(CH₂)ₙ—X;
n is an integer from 0 to 6;
X is selected from the group consisting of: —OH, —NH₂, and substituted or unsubstituted C₃-C₈ cycloalkyl;
R⁵ is selected from the group consisting of: substituted or unsubstituted aryl, substituted or unsubstituted heteroaryl, substituted or unsubstituted heterocyclyl, substituted or unsubstituted C₃-C₈ cycloalkyl, and —(CH₂)ₒ—Y;
o is an integer from 0 to 6;
Y is selected from the group consisting of: substituted or unsubstituted aryl, substituted or unsubstituted heteroaryl, substituted or unsubstituted heterocyclyl, and substituted or unsubstituted C₃-C₈ cycloalkyl;
R⁶ is selected from the group consisting of: —H, linear C₁-C₈ substituted or unsubstituted alkyl, branched C₁-C₈ substituted or unsubstituted alkyl, substituted or unsubstituted aryl, substituted or unsubstituted heteroaryl, substituted or unsubstituted heterocyclyl, substituted pyrrolidinyl, substituted or unsubstituted C₃-C₈ cycloalkyl, disubstituted cyclohexyl, cyclopentyl, substituted or unsubstituted C₅-C₁₂ bicycloalkyl, substituted or unsubstituted adamantyl, a methylene group represented by —(CH₂)_{q}—, and —(CH₂)ₚ-Z;
q is an integer from 1 to 3;
p is an integer from 0 to 6;
Z is selected from the group consisting of: substituted or unsubstituted aryl, substituted or unsubstituted heteroaryl, substituted or unsubstituted heterocyclyl, —N(R⁷R⁸), and —(CR⁹, R¹⁰R¹¹); R⁷ and R⁸ independently are selected from the group consisting of: —H, linear C₁-C₆ substituted or unsubstituted alkyl, and branched C₁-C₆ substituted or unsubstituted alkyl;
R⁹, R¹⁰ and R¹¹ independently are selected from the group consisting of: —H, linear C₁-C₈ substituted or unsubstituted alkyl, branched C₁-C₈ substituted or unsubstituted alkyl, substituted or unsubstituted aryl, and —N(R¹²R¹³);
R¹² and R¹³ independently are selected from the group consisting of: —H, linear C₁-C₆ substituted or unsubstituted alkyl, and branched C₁-C₆ substituted or unsubstituted alkyl, wherein when R⁶ is —(CH₂)_{q}—, then R¹⁷ and R¹⁹ are selected from the group consisting of —(CH₂)ₛ—, and —(CH₂)ₜ-A, in which both s and t are an integer from 1 to 3, and A is O or N; or R⁶ taken together with R¹⁷ forms a 5 to 8 membered ring; and when Z is substituted or unsubstituted aryl, substituted or unsubstituted heteroaryl, substituted or unsubstituted heterocyclyl, or —N(R⁷R⁸), then p is an integer from 1 to 6;
L is selected from the group consisting of: —NH(C═O)NH—, —NH(C═S)NH—, —NH(C═O)O—, —(C═O)—, —(C═O)O—, —SO—, —SO₂—, —SO₃—, NH(C═NH)NH—, —NR¹⁴—SO₂—, —NR¹⁴—SO—, —NR¹⁷—CO—, —SO₂—NR¹⁸—, and —CO—NR¹⁹—;
m is 0 or 1;
R¹⁴ is selected from the group consisting of: —H, linear C₁-C₆ substituted or unsubstituted alkyl, branched C₁-C₆ substituted or unsubstituted alkyl, —SO₂—R¹⁵, and —(CH₂)ᵣ—COOR¹⁶;
R¹⁵ is linear or branched C₁-C₆ alkyl;
r is an integer from 0 to 6;
R¹⁶ is selected from the group consisting of; —H, linear C₁-C₆ substituted or unsubstituted alkyl, and branched C₁-C₆ substituted or unsubstituted alkyl;
R¹⁷ is selected from the group consisting of: —H, linear C₁-C₆ substituted or unsubstituted alkyl, branched C₁-C₆ substituted or unsubstituted alkyl, and a methylene group represented by —(CH₂)ₛ—, wherein s is an integer from 1 to 3;
R¹⁸ is selected from the group consisting of: —H, linear C₁-C₆ substituted or unsubstituted alkyl, and branched C₁-C₆ substituted or unsubstituted alkyl;
wherein, when R⁶ and R¹⁷ are both a methylene group, then L-R⁶ may have the following the formula: 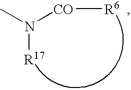
in which R⁶ and R¹⁷ together form a 5 to 8 membered ring;
R¹⁹ is selected from the group consisting of —H, linear C₁-C₆ substituted or unsubstituted alkyl, branched C₁-C₆ substituted or unsubstituted alkyl, and —(CH₂)ₜ-A, wherein t is an integer from 1 to 3, and A is N or O; wherein when R⁶ is —(CH₂)_{q} and R¹⁹ is (CH₂)ᵣ-A-, then L-R⁶ may have the following the formula: 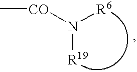
in which R⁶ and R¹⁹ together form a 5 to 8 membered ring.

16. The method of claim 1, further comprising administering a compound selected from the group consisting of a nonsteroidal anti-inflammatory drug, an anticonvulsant, an antidepressant and an opioid to said patient.

17. A composition comprising at least one CDK inhibitor and an analgesic agent, said analgesic agent having a mechanism of action other than inhibition of a CDK.

18. The composition of claim 17, wherein said at least one CDK inhibitor is a selective inhibitor of CDK9.

19. The composition of claim 17, wherein said at least one CDK inhibitor is selected from the class of 4,6-disubstituted aminopyrimidine derivatives.

20. A pharmaceutical composition comprising a composition of claim 17 and a pharmaceutically acceptable carrier, diluent or excipient.

21. A pharmaceutical composition comprising at least one CDK inhibitor and a pharmaceutically acceptable carrier, diluent or excipient.

22. A method of treating inflammatory and/or neuropathic pain, said method comprising administering an effective amount of a composition of claim 20 to a subject in need thereof.

23. A method of treating inflammatory and/or neuropathic pain, said method comprising administering an effective amount of a composition of claim 21 to a subject in need thereof.

24. The composition of claim 20, wherein said at least one inhibitor of a cyclin-dependent kinase is selected from the group consisting of:
Compound 1: N-{4-[6-(4-Methoxy-phenyl)-pyrimidin-4-ylamino]-phenyl}-4-methyl-benzenesulfonamide,
Compound 2: N-{4-[6-(3-Methoxy-phenyl)-pyrimidin-4ylamino]-phenyl}-4-methyl-benzenesulfonamide,
Compound 3: N-{5-[6-(4-Methoxy-phenyl)-pyrimidin-4-ylamino]-2-methyl-phenyl}-methanesulfonamide,
Compound 4: 4-Amino-N-{4-[6-(2-benzyloxy-phenyl)-pyrimidin-4-ylamino]-phenyl}-benzamide,
Compound 5: N-{4-[6-(2-Methoxy-phenyl)-pyrimidin-4ylamino]-phenyl}-4-methyl-benzenesulfonamide,
Compound 6: 4-Amino-N-{4-[6-(4-methoxy-phenyl)-pyrimidin-4-ylamino]-phenyl}-benzamide,
Compound 7: [6-(2-Benzyloxy-phenyl)-pyrimidin-4-yl]-(2-pyridin-4-yl-ethyl)-amine,
Compound 8: 4-Amino-N-{4-[6-(2-methoxy-phenyl)-pyrimidin-4-ylamino]-phenyl}-benzamide,
Compound 9: 1-{4-[6-(2-Methoxy-phenyl)-pyrimidin-4-ylamino]-phenyl}-pyrrolidin-2-one,
Compound 10: N-{4-[6-(2-methoxy-phenyl)-pyrimidin-4-ylamino]-phenyl}-acetamide,
Compound 11: N-{4-[6-(4-Hydroxy-phenyl)-pyrimidin-4ylamino]-phenyl}-4-methyl-benzenesulfonamide,
Compound 12: N-{5-[6-(3-Amino-phenyl)-pyrimidin-4ylamino]-2-methyl-phenyl}-methanesulfonamide,
Compound 13: [6-(3-Amino-phenyl)-pyrimidin-4-yl]-(2-pyridin-4-ylethyl)-amine,
Compound 14: 4-[6-(2-Methoxy-phenyl)-pyrimidin-4-ylamino]l-benzamide,
Compound 15: 4-[6-(2-Methoxy-phenyl)-pyrimidin-4-ylamino]l-benzoic acid methyl ester,
Compound 16: 4-Amino-N-{4-[6-(4-hydroxy-phenyl)-pyrimidin-4-ylamino]-phenyl}-benzamide,
Compound 17: 3-(4-{6-[4-Toluene-4-sulfonylamino)-phenylamino]-pyrimidin-4-yl}-phenyl)-propionic acid,
Compound 18: N-{4-[6-(2-Methoxy-phenyl)-pyrimidin-4ylamino]-phenyl}-4-methyl-N-propyl-benzenesulfonamide,
Compound 19: N-{4-[6-(2-Methoxy-phenyl)-pyrimidin-4-ylamino]-phenyl}-2,2-dimethyl-propionamide,
Compound 20: 2-Amino-N-{4-[6-(2-methoxy-phenyl)-pyrimidin-4-ylamino]-phenyl}-benzamide,
Compound 21: 4-Amino-N-{4-[6-(3-amino-phenyl)-pyrimidin-4-ylamino]-phenyl}-benzamide,
Compound 22: N-[6-(2-Methoxy-phenyl)-pyrimidin-4-yl]-benzene-1,4-diamine,
Compound 23: 4-isopropyl-N-{4-[6-(2-methoxy-phenyl)-pyrimidin-4ylamino]-phenyl}-benzenesulfonamide,
Compound 24: N-[4-(6-Chloro-pyrimidin-4-ylamino)phenyl]-4-methyl-benzenesulfonamide,
Compound 25: 4-Amino-N-[4-(6-chloro-pyrimidin-4ylamino)-phenyl]-benzamide,
Compound 26: N-[6-(2-Methox-phenyl)-pyrimidin-4-ylamino]-N-methyl-benzene-1,4-diamine,
Compound 27: [{4-[6-(4-Hydrox-phenyl)-pyrimidin-4-ylamino]-phenyl}-(toluene-4-sulfonyl)-amino]-acetic acid methyl ester,
Compound 28: [{4-[6-(2-Methoxy-phenyl)-pyrimidin-4-ylamino]-phenyl}-(toluene-4-sulfonyl)-amino]-acetic acid methyl ester,
Compound 29: (S)-2-{4-[6-(2-Methoxy-phenyl)-pyrimidin-4-ylamino]-phenylcarbamoyl}-piperidine-1-carboxylic acid tert-butyl ester,
Compound 30: (S)-Piperidine-2-carboxylic acid N-{4-[6-(2-methoxy-phenyl)-pyrimidin-4-ylamino]-phenyl}-amide
Compound 31: 4-Amino-N-{4-[6-(2,4-dimethoxy-phenyl)-pyrimidin-4-ylamino]-3-phenyl}-benzamide,
Compound 32: 4-Amino-N-{4-[6-styryl-pyrimidin-4-ylamino]-phenyl}-benzamide,
Compound 33: N-{4-[6-(2-Methoxy-phenyl)-pyrimidin-4yl-amino]-phenyl}-methanesulfon-amide,
Compound 34: Biphenyl-4-sulfonic acid -{4-[6-(2-methoxy-phenyl)-pyrimidin-4-ylamino]-phenyl}-amide,
Compound 35: 4-Amino-N-{4-[6-(5-isopropyl-2-methoxy-phenyl)-pyrimidin-4-ylamino]-phenyl}-benzamide,
Compound 36: Bicyclo[2.2.1)heptane-2-carboxylic acid {4-[6-(2-methoxyphenyl)-pyrimidin-4-ylamino]-phenyl}-amide,
Compound 37: N-{4-[6-(2-Methoxy-phenyl)-pyrimidin-4-ylamino]-phenyl}-3-methyl-2-phenyl-butyramide,
Compound 38: 1-Cyclohexyl-3-{4-[6-(2-methoxy-phenyl)-pyrimidin-4-ylamino]-phenyl}-urea,
Compound 39: 4-Amino-N-{4-[6-(5-chloro-2-methoxy-phenyl)-pyrimidin-4-ylamino]-phenyl}-benzamide,
Compound 40: E3-(3-{6-[4-(Toluene-4-sulfonylamino)-phenylamino]-pyrimidin-4-yl)phenyl}-acrylic acid,
Compound 41: Cyclohexanecarboxylic acid {4-[6-(2-methoxy phenyl)-pyrimidin-4-ylamino]-phenyl}-amide,
Compound 42: N-{-4-[6-(2-Methoxy-phenyl)-pyrimidin-4-ylamino]-phenyl}-3,3-dimethyl-butyramide,
Compound 43: 4-Amino-N-{4-[6-(cyclohexylmethyl-amino)-pyrimidin-4-ylamino]-phenyl}-benzamide,
Compound 44: N-Cyclohexyl-{4-[6-(2-methoxy-phenyl)-pyrimidin-4-ylamino]-benzamide,
Compound 45: 4-tert-Butyl-N-{4-[6-(2-methoxy-phenyl)-pyrimidin-4-ylamino]-phenyl}-benzamide,
Compound 46: 2-Dimethylamino-N-{4-[6-(2-methoxy-phenyl)-pyrimidin-4-ylamino]-phenyl}-acetamide,
Compound 47: (1-{4-[6-(2-methoxy-phenyl)-pyrimidin-4-ylamino]-phenyl-carbamoyl-cyclopentyl)-carbamic acid tert butyl ester
Compound 48: 2-({4-[6-(2-Methoxy-phenyl)pyrimidin-4-ylamino]-phenylcarbamoyl}-methyl)piperidine-1-carboxylic acid tert-butyl ester,
Compound 49: N-{4-[6-(2-Methoxy-phenyl)-pyrimidin-4-ylamino]-phenyl}-4-(4-methyl-piperazin-1-yl)-benzamide,
Compound 50: N-{4-[6-(2-Methoxy-phenyl)-pyrimidin-4-ylamino]-phenyl}-isonicotinamide,
Compound 51: 4-Amino-N-{4-[6-(2,6-dimethoxy-phenyl)-pyrimidin-4-ylamino]-phenyl}-benzamide,
Compound 52: {4-[6-(2-Methoxy-phenyl)-pyrimidin-4-ylamino]-N-phenyl-benzamide,
Compound 53: N-{4-[6-(2-Methoxy-phenyl)-pyrimidin-4-ylamino]-phenyl}-guanidine,
Compound 54: N-tert-Butyl-4-[6-(2-methoxy-phenyl)-pyrimidin-4-ylamino]-benzamide,
Compound 55: 4-Amino-N-{4-[6-(2-ethoxy-phenyl)-pyrimidin-4-ylamino]-phenyl}-benzamide,
Compound 56: 4-Amino-N-{4-[6-(2,3-dimethoxy-phenyl)-pyrimidin-4-ylamino]-phenyl}-benzamide,
Compound 57: 4-Amino-N-{4-[6-(2,5-dimethoxy-phenyl)pyrimidin-4-ylamino]-phenyl}-benzamide,
Compound 58: 4-Amino-N-{4-[6-(2-isopropoxy-phenyl)-pyrimidin-4-ylamino]-phenyl}-benzamide,
Compound 59: N-{4-[6-(2-Methoxy-phenyl)-pyrimidin-4-ylamino]-phenyl}-2-piperidine-2-yl-acetamide,
Compound 60: 4-Amino-N-{4-[6-(2-hydroxy-ethylamino)-pyrimidin-4-ylamino]-phenyl}-benzamide,
Compound 61: Adamantane-1-carboxylic acid-{4-[6-(2-methoxyphenyl)-pyrimidin-4-yl-amino]-phenyl}-amide,
Compound 62: (4-Benzoxazol-2-yl-phenyl)-[6-(2-methoxy-phenyl)-pyrimidin-4-yl]-amine,
Compound 63: [4-(1H-Benzimidazole-2-yl)-phenyl]-[6-(2-methoxy-phenyl)-pyrimidin-4-yl]-amine,
Compound 64: 3-Diethylamino-N-{4-[6-(2-methoxy-phenyl)-pyrimidin-4-ylamino]-phenyl}-propion amide,
Compound 65: (S)-1,2,3,4-Tetrahydro-isoquinoline-3-carboxylic acid-{4-[6-(2-methoxy-phenyl)-pyrimidin-4-ylamino]phenyl}amide,
Compound 66: 1-Amino-cyclohexane carboxylic acid-{4-[6-(2-methoxy-phenyl)-pyrimidin-4-ylamino]-phenyl}-amide,
Compound 67: 4-Amino-N-[4-(6-pyridin-4-yl-pyrimidin-4-ylamino)-phenyl]-benzamide,
Compound 68: 1-Amino-cyclopentanecarboxylic acid {4-[6-(2-methoxy-phenyl)-pyrimidin-4-ylamino]-phenyl}-amide,
Compound 69: (R)-Piperidine-2-carboxylic acid {4-[6-(2-methoxy-phenyl)-pyrimidin-4-ylamino]-phenyl}-amide,
Compound 70: 1-Methyl-4H-imidazole-4-carboxylic acid {4-[6-(2-methoxy-phenyl)-pyrimidin-4-ylamino]-phenyl}amide,
Compound 71: N-{4-[6-(2-Methoxy-phenyl)-pyrimidin-4-ylamino]-phenyl}-2-phenyl-actetamide,
Compound 72: N-[4-(6-Chloro-pyrimidin-4-ylamino)-phenyl]-2,2-dimethyl-propionamide,
Compound 73: 2,2-Dimethyl-N-[4-(6-pyridin-3-yl-pyrimidin-4-ylamino)-phenyl]-propionamide,
Compound 74: 2,2-Dimethyl-N-{4-[6-(1-methyl-piperidin-4-ylamino)-pyrimidin-4-ylamino]-phenyl}-propionamide,
Compound 75: 3-{6-[4-(2,2-Dimethyl-propionylamino)phenylamino]-pyrimidin-4-yl}benzoic acid,
Compound 76: 4-Amino-N-[4-(6-phenyl-pyrimidin-4-ylamino)-phenyl]-benzamide,
Compound 77: 4-Amino-N-[4-(6-thiophen-2-yl-pyrimidin-4-ylamino)-phenyl]-benzamide
Compound 78: 2,2-Dimethyl-N-{4-[6-(4-methyl-piperazin-1-ylamino)-pyrimidin-4-ylamino]-phenyl}-propionamide,
Compound 79: N-{4-[6-(2-Amino-ethylamino)-pyrimidin-4-ylamino]-phenyl}-2,2-dimethyl-propionamide,
Compound 80: N-{4-[6-(3-Hydroxy-propylamino)-pyrimidin-4-ylamino]-phenyl}-2,2-dimethyl-propionamide,
Compound 81: (S)-2-Amino-N-{4-[6-(2-methoxy-phenyl)-pyrimidin-4-ylamino]-phenyl}-2-phenyl-acetamide,
Compound 82: (S)—N-{4-[6-(2-methoxy-phenyl)-pyrimidin-4-ylamino]-phenyl}-2-methylamino-2-phenyl-acetamide,
Compound 83: (R,R)/(SS)—N-(2-Amino-cyclohexyl)-4-[6-(2-methoxy-phenyl)-pyrimidin-4-ylamino]-benzamide,
Compound 84: Benzothiazole-2-carboxylic acid -{4-[6-(2-methoxy-phenyl)-pyrimidin-4-ylamino]-phenyl}-amide,
Compound 85: N-{4-[6-(2-Benzyloxy-phenyl)-pyrimidin-4-ylamino]-phenyl}-2,2-dimethyl-propionamide,
Compound 86: 4-[6-(2-Methoxy-phenyl)-pyrimidin-4-ylamino]-N-piperidin-3-yl-benzamide.
Compound 87: 1-Methyl-piperidine-3-carboxylic acid {4-[6-(2-methoxy-phenyl)-pyrimidin-4-ylamino]-phenyl}-amide.
Compound 88: 4-(6-Chloro-pyrimidin-4-ylamino)-N-cyclohexyl-benzamide,
Compound 89: 1-Methyl-piperidine-4-carboxylic acid {4-[6-(2-methoxy-phenyl)-pyrimidin-4-yl]-amino]-phenyl}-amide,
Compound 90: (S)-Azetidine-2-carboxylic acid {4-[6-(2-methoxy-phenyl)-pyrimidin-4-yl]-amino]-phenyl}-amide,
Compound 91: (R)-Pyrrolidine-2-carboxylic acid {4-[6-(2-methoxy-phenyl)-pyrimidin-4-yl]-amino]-phenyl}-amide,
Compound 92: [6-(4-Methoxy-phenyl)-pyrimidin-4-yl]-(2-pyridin-4-yl-ethyl)-amine,
Compound 93: [6-(2-Methoxy-phenyl)-pyrimidin-4-yl]-(2-pyridin-4-yl-ethyl)-amine,
Compound 94: 2-[6-(2-Pyridin-4-yl-ethylamino)-pyrimidin-4-yl]-phenol,
Compound 95: 4-[6-(2-Benzyloxy-phenyl)pyrimidin-4-ylamino]-benzamide,
Compound 96: N-(4-{[6-(2-Methoxy-phenyl)-pyrimidin-4-yl]-methyl-amino}phenyl)-4-methyl-benzenesulfonamide,
Compound 97: 4-Amino-N-{4-[2-amino-6-(2-methoxy-phenyl)-pyrimidin-4-yamino]-phenyl}-benzamide,
Compound 98: Quinoline-2-carboxylic acid {4-[6-(2-methoxy-phenyl)-pyrimidin-4-ylamino]-phenyl}-amide,
Compound 99: 6-(2-Isopropoxy-phenyl)pyrimidin-4-yl]-(2-pyridinyl-ethyl)-amine,
Compound 100: N-{5-[6-(3-Methoxy-phenyl)-pyrimidin-4-ylamino]-2-methyl-phenyl}-methane sulfonamide,
Compound 101: 2-Dimethylamino-N-{4-[6-(2-methoxy-phenyl)-pyrimidin-4-ylamino]-phenyl}-2-phenyl-acetamide,
Compound 102: 3-Amino-N-{-4-[6-(2-methoxy-phenyl)-pyrimidin-4-ylamino]-phenyl}-propion-amide,
Compound 103: 4-Amino-N-(4-{6-[2-(3-amino-propoxy phenyl]-pyrimidin-4-ylamino}phenyl)-benzamide,
Compound 104: N-{3-[6-(3-Methanesulfonylamino-4-methyl-phenylamino)-pyrimidin-4-yl]-phenyl}-acetamide,
Compound 105: N-{5-[6-(3-Hydroxy-phenyl)-pyrimidin-4-ylamino]-2-methyl-phenyl}methane-sulfonamide,
Compound 106: N-[2-Methyl-5-(6-phenyl-pyrimidin-4-ylamino)-phenyl]-methanesulfonamide,
Compound 107: N-{2-Methyl-5-[6-(3-trifluoromethyl-phenyl)-pyrimidin-4-ylamino]-phenyl}-methanesulfonamide,
Compound 108: N-{5-[6-(3-Methanesulfonylamino-phenyl)-pyrimidin-4-ylamino]-2-methyl-phenyl}-methanesulfonamide,
Compound 109: N-{5-[6-(3-Amino-phenyl)-pyrimidin-4-ylamino]-2-methyl-phenyl}-benzene-sulfonamide,
Compound 110: N-[5-([4,5′]Bipyrimidinyl-6-ylamino)-2-methyl-phenyl]-methanesulfonamide,
Compound 111: 1-Benzo [1,3] dioxol-5-yl-3-{4-[6-(2-methoxy-phenyl)-pyrimidin-4-ylamino]-phenyl}-urea,
Compound 112: 1-{4-[6-(2-Methoxy-phenylpyrimidin-4-ylamino]-phenyl}-3-(4-methyl-benzyl)-urea,
Compound 113: 1-tert-Butyl-3-{4-[6-(2-methoxy-phenyl)-pyrimidin-4-ylamino]-phenyl}-urea,
Compound 114: 2,2-Dimethyl-N-{4-[6-(2-trifluoromethyl-phenyl)-pyrimidin-4-ylamino]-phenyl}-propionamide,
Compound 115: 3-[6-(2-Methoxy-phenyl)-pyrimidin-4-ylamino]-benzamide,
Compound 116: Propane-1-sulfonic acid {5-[6-(3-amino-phenyl)-pyrimidin-4-ylamino]-2-methyl-phenyl}-amide,
Compound 117: 4-[6-(3-Amino-phenyl)-pyrimidin-4-ylamino]-benzenesulfonamide,
Compound 118: N-{4-[6-(2-Methoxy-phenyl)-pyrimidin-4-ylamino]-phenyl}-2-methyl-2-methylamino-propionamide,
Compound 119: N-{4-[6-(2-Methoxy-phenyl)-pyrimidin-4-amino]-3-methyl-phenyl}-2,2-dimethyl-propionamide,
Compound 120: N-{5-[6-(3-Amino-phenyl)-pyrimidin-4-ylamino]-2-benzyloxy-phenyl}-methanesulfonamide,
Compound 121: N-{3-[6-(3-Amino-phenyl)-pyrimidin-4-ylamino]-phenyl}-methanesulfon-amide,
Compound 122: N-{3-[6-(2-Methoxy-phenyl)-pyrimidin-4-ylamino]-phenyl}-2,2-dimethyl-propionamide,
Compound 123: N*1*-[6-(2-Methoxy-phenyl)-pyrimidin-4-yl]-2-methyl-benzene-1,4-diamine,
Compound 124: N-[6-(2-Methoxy-phenyl)-pyrimidin-4-yl]-benzene-1,3-diamine,
Compound 125: 4-[6-(2-Methoxy-phenyl)-pyrimidin-4-ylamino]-N-(4-morpholin-4-yl-phenyl)-benzamide,
Compound 126: 2,2-Dimethyl-N-{4-[6-(2-vinyl-phenyl)-pyrimidin-4-ylamino]-phenyl}-propionamide,
Compound 127: N-{4-[6-(2-Fluoro-phenyl)-pyrimidin-4-ylamino]-phenyl}-2,2-dimethyl-propionamide,
Compound 128: (S)-Piperidine-2-carboxylic acid {3-[6-(2-methoxy-phenyl)-pyrimidin-4-ylamino]-phenyl}-amide,
Compound 129: 2-Oxo-2H-chromene-3-carboxylic acid {4-[6-(2-methoxy-phenyl)-pyrimidin-4-ylamino]-phenyl}-amide,
Compound 130: Benzo [1,3] dioxole-5-carboxylic acid {4-[6-(2-methoxy phenyl)-pyrimidin-4-ylamino]-phenyl}-amide,
Compound 131: N-{4-[6-(2-Ethyl-phenyl)-pyrimidin-4-ylamino]-phenyl}-2,2-dimethyl-propion-amide,
Compound 132: N-[4-(6-Biphenyl-2-yl-pyrimidin-4-ylamino)-phenyl]-2,2-dimethyl-propion-amide,
Compound 133: 1H-Indole-3-carboxylic acid {4-[6-(2-methoxy-phenyl)-pyrimidin-4-ylamino]-phenyl}-amide,
Compound 135: N-((1R,2R)/(1S,2S)-2-Hydroxy-cyclohexyl)-4-[6-(2-methoxy-phenyl)-pyrimidin-4-ylamino]-benzamide,
Compound 136: N-(4-Hydroxy-phenyl)-4-[6-(2-methoxy-phenyl)-pyrimidin-4-ylamino]-benzamide,
Compound 137: N-(4-Isopropyl-phenyl)-4-[6-(2-methoxy-phenyl)-pyrimidin-4-ylamino]-benzamide,
Compound 138: 1H-Benzoimidazole-5-carboxylic acid {4-[6-(2-methoxy-phenyl)-pyrimidin-4-ylamino]-phenyl}-amide,
Compound 139: 1-Hydroxy-naphthalene-2-carboxylic acid {4-[6-(2-methoxy-phenyl)-pyrimidin-4-ylamino]-phenyl}-amide,
Compound 140: (2S,3S)-2-Amino-3-methyl-pentanoic acid {4-[6-(2-methoxy-phenyl)-pyrimidin-4-ylamino]-phenyl}-amide,
Compound 141: 1H-Indazole-3-carboxylic acid {4-[6-(2-methoxy-phenyl)-pyrimidin-4-ylamino]-phenyl}-amide,
Compound 142: Quinoline-8-sulfonic acid {5-[6-(3-amino-phenyl)-pyrimidin-4-ylamino]-2-methyl-phenyl}-amide,
Compound 143: (S)-2-Amino-N-{4-[6-(2-methoxy-phenyl)-pyrimidin-4-ylamino]-phenyl}-3-methyl-butyramide,
Compound 144: 1-Methyl-1H-imidazole-4-sulfonic acid {5-[6-(3-amino-phenyl)-pyrimidin-4-ylamino]-2-methyl-phenyl}-amide,
Compound 145: 3-Hydroxy-naphthalene-2-carboxylic acid {4-[6-(2-methoxy-phenyl)-pyrimidin-4-ylamino]-phenyl}-amide,
Compound 146: 2-Amino-N-{4-[6-(2-methoxy-phenyl)-pyrimidin-4-ylamino]-phenyl}-2-naphthalen-2-yl-acetamide,
Compound 147: {4-[6-(2-Methoxy-phenyl)-pyrimidin-4-ylamino]-phenyl}-morpholin-4-yl-methanone,
Compound 148: N-((1S,2R)/(1R,2S)-2-Aminocyclohexyl)-4-[6-(2-methoxy-phenyl)-pyrimidin-4-ylamino]-benzamide,
Compound 149: 4-Amino-N-{4-[6-(2-methoxy-phenyl-5-methyl-pyrimidin-4-ylamino]-phenyl}-benzamide,
Compound 150: 3-[6-(2-Methoxy-phenyl)-pyrimidin-4-ylamino]-benzenesulfonamide,
Compound 151: 4-Amino-N-{4-[6-(2-hydroxy-phenyl)-pyrimidin-4-ylamino]-phenyl}-benzamide,
Compound 152: N-[6-(2-Methoxy-phenyl)-5-methyl-pyrimidin-4-yl]-benzene-1,4-diamine,
Compound 153: Propane-2-sulfonic acid {4-[6-(2-methoxy-phenyl)-pyrimidin-4-ylamino]-phenyl}-amide,
Compound 154: Propane-1-sulfonic acid {4-[6-(2-methoxy-phenyl)-pyrimidin-4-ylamino]-phenyl}-amide,
Compound 155: N-{4-[6-(2-Methoxy-phenyl)-pyrimidin-4-ylamino]-phenyl}-benzene sulfonamide,
Compound 156: N-{5-[6-(2-Benzyloxy-phenyl)-pyrimidin-4-ylamino]-2-methyl-phenyl}-methanesulfonamide,
Compound 157: N-{5-[6-(3-Dimethylamino-phenyl)-pyrimidin-4-ylamino]-2-methyl-phenyl}-methanesulfonamide,
Compound 158: N-{5-[6-(2-Isopropoxy-phenyl)-pyrimidin-4-ylamino]-2-methyl-phenyl}-methanesulfonamide,
Compound 159: N-Bis-propane-1-sulfonic acid-{4-[6-(2-methoxy-phenyl)-5-methyl-pyrimidin-4-ylamino]-phenyl}-amide,
Compound 160: Propane-1-sulfonic acid {4-[6-(2-methoxy-phenyl)-5-methyl-pyrimidin-4-ylamino]-phenyl}-amide,
Compound 161: N-(2-Aminocyclohexyl)-4-[6-(4-methoxy-phenyl)-pyrimidin-4-ylamino]-benzamide,
Compound 162: N-{5-[6-(2-Methoxy-phenyl)-pyrimidin-4-ylamino]-2-methyl-phenyl}methanesulfonamide,
Compound 163: N-{5-[6-(3-Cyano-phenyl)-pyrimidin-4-ylamino]-2-methyl-phenyl}-methane sulfonamide,
Compound 164: (S)-Piperidine-2-carboxylic acid {3-[6-(2-benzyloxy-phenyl)-pyrimidin-4-ylamino]-phenyl}-amide,
Compound 165: N-{5-[6-(3-Formyl-phenyl)-pyrimidin-4-ylamino]-2-methyl-phenyl}-methane sulfonamide,
Compound 166: N-{5-[6-(2-Hydroxymethyl-phenyl)-pyrimidin-4-ylamino]-2-methyl-phenyl}-methanesulfonamide,
Compound 167: (S)-Piperidine-2-carboxylic acid {3-[6-(4-methoxy-phenyl)-pyrimidin-4-ylamino]-phenyl}-amide,
Compound 168: (S)-Piperidine-2-carboxylic acid {3-[6-(3-formyl-phenyl)-pyrimidin-4-ylamino]-phenyl}-amide,
Compound 169: (S)-Piperidine-2-carboxylic acid {3-[6-(3-dimethylamino-phenyl)-pyrimidin-4-ylamino]-phenyl}-amide,
Compound 170: (S)-Piperidine-2-carboxylic acid {3-[6-(2-hydroxymethyl-phenyl)-pyrimidin-4-ylamino]-phenyl}-amide,
Compound 171: (S)-Piperidine-2-carboxylic acid {3-[6-(2-methoxy-pyridin-3-yl)-pyrimidin-4-ylamino]-phenyl}-amide,
Compound 172: (S)-Piperidine-2-carboxylic acid {3-[6-(6-methoxy-pyridin-3-yl)-pyrimidin-4-ylamino]-phenyl}-amide,
Compound 173: (S)-Piperidine-2-carboxylic acid {3-[6-(4-benzyloxy-phenyl)-pyrimidin-4-ylamino]-phenyl}-amide,
Compound 174: (S)-Piperidine-2-carboxylic acid {3-[6-(4-phenoxy-phenyl)-pyrimidin-4-yl-amino]-phenyl}-amide
Compound 175: N-{5-[6-(4-Hydroxymethyl-phenyl)-pyrimidin-4-ylamino]-2-methyl-phenyl}-methanesulfonamide, and
Compound 176: N-{5-[6-(2-Methoxy-pyridin-3-yl)-pyrimidin-4-ylamino]-2-methyl-phenyl}-methanesulfonamide.
Compound 177: (S)-Piperidine-2-carboxylic acid {4-[6-(4-acetylamino-phenyl)-pyrimidin-4-ylamino]-phenyl}-amide,
Compound 178: (S)-Piperidine-2-carboxylic acid {4-[6-(3-methanesulfonylamino-phenyl)-pyrimidin-4-ylamino]-phenyl}-amide,
Compound 179: (S)-Piperidine-2-carboxylic acid {4-[6-(3-acetyl-phenyl)-pyrimidin-4-ylamino]-phenyl}-amide,
Compound 180: (S)-Piperidine-2-carboxylic acid {4-[6-(4-cyclopentylcarbamoyl-phenyl)-pyrimidin-4-ylamino]-phenyl}-amide,
Compound 181: N-{5-[6-(2-Hydroxy-phenyl)-pyrimidin-4-ylamino]-2-methyl-phenyl}-methanesulfonamide,
Compound 182: (E)-3-{3-[6-(3-Methanesulfonylamino-4-methyl-phenyl-amino)-pyrimidin-4-yl]-phenyl}-acrylic acid methyl ester,
Compound 183: N-{5-[6-(3-Hydroxymethyl-phenyl)pyrimidin-4-ylamino]-2-methyl-phenyl}-methanesulfonamide,
Compound 184: N-Butyl-3-[6-(2-methoxy-phenyl)-pyrimidin-1-ylamino]-benzenesulfonamide,
Compound 185: (3-Methanesulfonyl-phenyl)-[6-(2-methoxy-phenyl)-pyrimidin-4-yl]-amine,
Compound 186: (S)-Piperidine-2-carboxylic acid {4-[6-(2,3-dimethoxy-phenyl)-pyrimidin-4-ylamino]-phenyl}-amide,
Compound 187: (S)-Piperidine-2-carboxylic acid {4-[6-(2,4-dimethoxy-phenyl)-pyrimidin-4-ylamino]-phenyl}-amide,
Compound 188: (S)-Piperidine-2-carboxylic acid {4-[6-(2-isopropoxy phenyl)-pyrimidin-4-ylamino]-phenyl}-amide,
Compound 189: (S)-Piperidine-2-carboxylic acid {4-[6-(2-methylsulfanyl-phenyl)-pyrimidin-4-ylamino]-phenyl}-amide,
Compound 190: (S)-Piperidine-2-carboxylic acid {4-[6-(2-trifluoromethoxy-phenyl)-pyrimidin-4-ylamino]-phenyl}-amide,
Compound 191: (S)-Piperidine-2-carboxylic acid {4-[6-(5-acetyl-thiophen-2-yl)-pyrimidin-4-ylamino]-phenyl}-amide,
Compound 192: (S)-Piperidine-2-carboxylic acid {4-[6-(2-chloro-phenyl)-pyrimidin-4-ylamino]-phenyl}-amide,
Compound 193: (S)-Piperidine-2-caboxylic acid {4-[6-(3-hydroxymethyl-phenyl)-pyrimidin-4-ylamino]-phenyl}-amide,
Compound 194: N-(1S,2S)/(1R,2R)-2-Amino-cyclohexyl)-4-[6-(3-hydroxy-phenyl)-pyrimidin-4-ylamino]-benzamide,
Compound 195: N-((1S,2S)/(1R,2R)-2-Amino-cyclohexyl)-4-[6-(3-methanesulfonyl-aminophenyl)-pyrimidin-4-ylamino]-benzamide,
Compound 196: 4-[6-(2-Acetylamino-phenyl)-pyrimidin-4-ylamino]-N-((1S,2S)/(1R,2R)-2-amino-cyclohexyl)-benzamide,
Compound 197: N-{5-[6-(2-Methoxymethyl-phenyl)-pyrimidin-4-ylamino]-2-methyl-phenyl}-methane-sulfonamide,
Compound 198: N-((1R,2R)/(1S,2S)-2-Amino-cyclohexyl)-4-[6-(2-benzyloxy-phenyl)-pyrimidin-4-yl-amino]-benzamide,
Compound 199: N-((1R,2R)/(1S,2S)-2-Amino-cyclohex-yl)-4-[6-(2-isopropoxy-phenyl)-pyrimidin-4-ylamino]-benzamide,
Compound 200: 4-Amino-N-{4-[6-(4-methoxy-phenyl)-5-methyl-pyrimidin-4-ylamino]-phenyl}-benzamide,
Compound 201: 3-{6-[4-((1R,2R)/(1S,2S)-2-Amino-cyclohexylcarbamoyl)-phenylamino]-pyrimidin-4-yl}-benzoic acid methyl ester,
Compound 202: (S)-Piperidine-2-carboxylic acid {4-[6-(4-methoxy-phenyl pyrimidin-4-ylamino]-phenyl}-amide,
Compound 203: (S)-Piperidine-2-carboxylic acid {4-[6-(3-methoxymethyl-phenyl)-pyrimidin-4-ylamino]-phenyl}-amide,
Compound 204: N-[6-(2-Methoxy-phenyl)-2-methyl-pyrimidin-4-yl]-benzene-1,4-diamine,
Compound 205: N-[6-(4-Methoxy-phenyl)-2-methyl-pyrimidin-4-yl]-benzene-1,4-diamine,
Compound 206: N-((1R,2R)/(1S,2S)-2-Amino-cyclohexyl)-4-[6-(3-amino-phenyl)-pyrimidin-4-ylamino]-benzamide,
Compound 207: 4-[6-(3-Acetylamino-phenyl)-pyrimidin-4-ylamino]-N-((1R,2R)/(1S,2S)-2-amino-cyclohexyl)-benzamide,
Compound 208: N-((1R,2R)/(1S,2S)-2-Amino-cyclohexyl)-4-[6-(4-benzyloxy-phenyl)-pyrimidin-4-ylamino]-benzamide,
Compound 209: N-((1R,2R)/(1S,2S)-2-Amino-cyclohexyl)-4-[6-(3-cyano-phenyl)-pyrimidin-4-ylamino]-benzamide,
Compound 210: N-((1R,2R)/(1S,2S)-2-Amino-cyclohexyl)-4-[6-(2-methoxymethyl-phenyl)-pyrimidin-4-ylamino]-benzamide,
Compound 211: (S)-Piperidine-2-carboxylic acid {4-[6-(3-dimethylaminomethyl-phenyl)-pyrimidin-4-ylamino]-phenyl}-amide,
Compound 212: N-((1R,2R)/(1S,2S2-Amino-cyclohexyl)-4-(6-quinolin-3-yl-pyrimidin-4-ylamino)-benzamide,
Compound 213: N-((1R,2R)/(1S,2S)-2-Amino-cyclohexyl)-4-(2′-methoxy-[4,5′]bipyrimidinyl-6-ylamino)-benzamide,
Compound 214: 3-[6-(3-Amino-phenyl)-pyrimidin-4-ylamino]-benzenesulfonamide,
Compound 215: 3-[6-(4-Methoxy-phenyl)-pyrimidin-4-ylamino]-benzenesulfonamide,
Compound 216: (R,R)—N-(2-Amino-cyclohexyl)-4-[6-(2-hydroxymethyl-phenyl)-pyrimidin-4-ylamino]-benzamide,
Compound 217: N-(2-Diethylamino-ethyl)-4-[6-(2-methoxy-phenyl)-pyrimidin-4-ylamino]-benzamide,
Compound 218: (R,R)—N-(2-Amino-cyclohexyl)-4-[6-(2-hydroxy-phenyl)-pyrimidin-4-ylamino]-benzamide,
Compound 219: (R,R)—N-(2-Amino-cyclohexyl)-4-[6-(2-methoxy-pyridin-3-yl)-pyrimidin-4-ylamino]-benzamide,
Compound 220: (R,R)—N-(2-Amino-cyclohex)-4-[6-(5-dimethylaminomethyl-pyridin-3-yl)-pyrimidin-4-ylamino]-benzamide,
Compound 221: (R,R)-5-{6-[4-(2-Amino-cyclohexylcarbamoyl)-phenylamino]-pyrimidin-4-yl}-pyridine-2-carboxylic acid dimethylamide,
Compound 222: (R,R)—N-(2-Amino-cyclohexyl)-4-[6-(methylsulfanyl-pyridin-3-yl)-pyrimidin-4-ylamino]-benzamide,
Compound 223: (R,R)—N-(2-Amino-cyclohexyl)-4-[6-(5-aminomethyl-pyridin-3-yl)-pyrimidin-4-ylamino]-benzamide,
Compound 224: (R,R)—N-(2-Amino-cyclohexyl)-4-[6-(4-methylsulfanyl-pyridin-3-yl)-pyrimidin-4-ylamino]-benzamide,
Compound 225: N-(2-Amino-cyclohexyl)-4-[6-(5-hydroxymethyl-pyridin-3-yl)-pyrimidin-4-ylamino]-benzamide,
Compound 226: rac-4-[6-(2-Methoxy-phenyl)-pyrimidin-4-ylamino]-N-pyrrolidin-3-yl-benzamide,
Compound 227: (R,R)—N-(2-Amino-cyclohexyl)-4-[6-(5-dimethyamino-pyridin-3-yl)-pyrimidin-4-ylamino]-benzamide,
Compound 228: (R,R)-4-[6-(5-Acetyl-thiophen-2-yl)-pyrimidin-4-ylamino]-N-(2-amino-cyclohexyl)-benzamide,
Compound 229: [6-(2-Methoxy-phenyl)-pyrimidin-4-yl]-[3-(piperidine-1-sulfonyl)-phenyl]-amine N-(2-diethylamino-ethyl)-benzamide,
Compound 230: (R,R)-4-[6-(2-Acetyl-phenyl)-pyrimidin-4-ylamino]-N-(2-amino-cyclohexyl)-benzamide,
Compound 231: 4-[6-(2-Methoxy-phenyl)-pyrimidin-4-ylamino]-N-pyridin-3-yl-benzamide,
Compound 232: N-(1-Acetyl-piperidin-3-yl)-4-[6-(2-methoxy-phenyl)-pyrimidin-4-ylamino]-benzamide,
Compound 233: (R,R)—N-(2-Amino-cyclohexyl)-4-[6-(2-dimethylamino-phenyl)-pyrimidin-4-ylamino]-benzamide,
Compound 234: 4-{4-[6-(2-Methoxy-phenyl)-pyrimidin-4-ylamino]-benzoylamino}-pyrrolidine-1,2-dicarboxylic acid 1-tert-butyl ester 2-methyl ester,
Compound 235: 2-Chloro-5-[6-(2-methoxy-phenyl)-pyrimidin-4-ylamino]-benzenesulfonamide,
Compound 236: [6-(2-Methoxy-phenyl)-pyrimidin-4-yl]-[3-(piperidine-1-sulfonyl)-phenyl]-amine,
Compound 237: N-Allyl-3-[6-(2-methoxy-phenyl)-pyrimidin-4-ylamino]-benzenesulfonamide,
Compound 238: N-Benzyl-3-[6-(2-methoxy-phenyl)-pyrimidin-4-ylamino]-benzenesulfonamide,
Compound 239: [6-(2-Methoxy-phenyl)-pyrimidin-4-yl]-[3-(pyrrolidine-1-sulfonyl)-phenyl]-amine,
Compound 240: [6-(2-Methoxy-phenyl)-pyrimidinyl-4-yl]-[3-(morpholine-4-sulfonyl)-phenyl]-amine,
Compound 241: 3-[6-(2-Methoxy-phenyl)-pyrimidin-4-ylamino]-N-methyl-benzenesulfonamide,
Compound 242: N-[6-(2-Methoxy-phenyl)-pyrimidin-4-yl]-N-(3-sulfamoyl-phenyl)-acetamide,
Compound 243: N,N-Diallyl-3-[6-(2-methoxy-phenyl)-pyrimidin-4-ylamino]-benzenesulfonamide,
Compound 244: 3-[6-(2-Benzyloxy-phenyl)-pyrimidin-4-ylamino]-benzenesulfonamide,
Compound 245: [6-(2-Methoxy-phenyl)-pyrimidin-4-yl]-[4-(4-nitro-benzenesulfonyl)-phenyl]-amine,
Compound 246: [6-(2-Methoxy-phenyl)-pyrimidin-4-yl]-(4-trifluoromethanesulfonyl-phenyl)-amine,
Compound 247: (4-Methanesulfonyl-phenyl)-[6-(2-methoxy-phenyl)-pyrimidin-4-yl]-amine,
Compound 248: N-(3,4-Dimethyl-isoxazol-5-yl)-4-[6-(2-methoxy-phenyl)-pyrimidin-4-ylamino]-benzenesulfonamide,
Compound 249: 4-[6-(2-Methoxy-phenyl)-pyrimidin-4-ylamino]-N-propyl-benzenesulfonamide,
Compound 250: 4-[6-(2-Methoxy-phenyl)-pyrimidin-4-ylamino]-benzenesulfonamide,
Compound 251: 4-[6-(2-Methoxy-phenyl)-pyrimidin-4-ylamino]-N,N-dimethyl-benzenesulfonamide,
Compound 252: N-(2-Methoxy-ethyl)-4-[6-(2-methoxy-phenyl)-pyrimidin-4-ylamino]-benzenesulfonamide,
Compound 253: [6-(2-Benzyloxy-phenyl)pyrimidin yl]-(3-methanesulfonyl-phenyl)-amine,
Compound 254: 2-[6-(3-Methanesulfonyl-phenylamino)-pyrimidin-4-yl]-phenol,
Compound 255: [6-(3-Amino-phenyl)-pyrimidin-4-yl]-(3-methanesulfonyl-phenyl)-amine,
Compound 256: 5-[6-(2-Methoxy-phenyl)-pyrimidin-4-ylamino]-2-methyl-benzenesulfonic acid,
Compound 257: 2-{3-[6-(2-Methoxy-phenyl)-pyrimidin-4-ylamino]-benzenesulfonyl}-ethanol,
Compound 258: (2-Fluoro-5-methanesulfonyl-phenyl)-[6-(2-methoxy-phenyl)-pyrimidin-4-yl]-amine,
Compound 259: [6-(2-Amino-phenyl)-pyrimidin-4-yl]-(3-methanesulfonyl-phenyl)-amine,
Compound 260: [6-(2-Methoxy-phenyl)-pyrimidin-4-yl]-(3-trifluoromethanesulfonyl-phenyl)-amine,
Compound 261: (3-Methanesulfonyl-phenyl)-[6-(2-Phenoxy-phenyl)-pyrimidin-4-yl]-amine,
Compound 262: [6-(2-Butoxy-phenyl)-pyrimidin-4-yl]-(3-methanesulfonyl-phenyl)-amine,
Compound 263: (3-Ethenesulfonyl-phenyl-[6-(2-methoxy-phenyl)-pyrimidin-4-yl]-mine,
Compound 264: (S)-Piperidine-2-carboxylic acid {4-[6-(4-methylsulfanyl-phenyl)-pyrimidin-4-ylamino]-phenyl}-amide,
Compound 265: 2-Chloro-4-[6-(2-methoxy-phenyl)-pyrimidin-4-ylamino]-benzoic acid methyl ester,
Compound 266: [6-(2-Methoxy-phenyl)-pyrimidin-4-yl]-(4-phenoxy-benzyl)-amine,
Compound 267: 4-[6-(2-Methoxy-phenyl)-pyrimidin-4-ylamino]-3-methyl-benzoic acid methyl ester,
Compound 268: [6-(3-Amino-phenyl)-pyrimidin-4-yl]-(1-methanesulfonyl-2,3-dihydro-1H-Indol-6-yl)-amine,
Compound 269: 3-[6-(2-Methoxy-phenyl)-pyrimidin-4-ylamino]-piperidine-1-carboxylic acid tertbutyl ester,
Compound 270: {4-[6-(2-Methoxy-phenyl)-pyrimidin-4-ylamino]-phenyl}-acetic acid,
Compound 271: (1H-Indazol-6-yl)-[6-(2-methoxy-phenyl)-pyrimidin-4-yl]-amine,
Compound 272: 1-{4-[6-(2-Methoxy-phenyl)-pyrimidin-4-ylamino]-phenyl}-butan-1-one,
Compound 273: [6-(2-Methoxy-phenyl)-pyrimidin-4-yl]-piperidin-3-yl-amine,
Compound 274: {4-[6-(2-Methoxy-phenyl-pyrimidin-4-ylamino]-phenyl}-phenyl-methanone,
Compound 275: N-[6-(2-Methoxy-phenyl)-pyrimidin-4-yl]-N′-phenyl-benzene-1,3-diamine,
Compound 276: (3-[1,3]Dioxan-2-yl-phenyl)-[6-(2-methoxy-phenyl)-pyrimidin-4-yl]-amine,
Compound 277: (3-Methoxy-phenyl)-[6-(2-methoxy-phenyl)-pyrimidin-4-yl]-amine,
Compound 278: (4-Methoxy-phenyl)-[6-(2-Methoxy-phenyl)-pyrimidin-4-yl]-amine,
Compound 279: N-[6-(2-Methoxy-phenyl)-pyrimidin-4-yl]-N′-phenyl-benzene-1,4-diamine,
Compound 280: [6-(2-Methoxy-phenyl)-pyrimidin-4-yl]-(4-morpholin-4-yl-phenyl)amine,
Compound 281: (2-Fluoro-phenyl)-[6-(2-methoxy-phenyl)-pyrimidin-4-yl]-amine,
Compound 282: (1-Benzyl-piperidin-4-yl)-[6-(2-Methoxy-phenyl)-pyrimidin-4-yl]-amine,
Compound 283: (4-Butyl-phenyl)-[6-(2-methoxy-phenyl)-pyrimidin-4-yl]-amine,
Compound 284: [6-(2-Methoxy-phenyl)-pyrimidin-4-yl]-(4-phenoxy-phenyl)-amine,
Compound 285: 4-{[6-(2-Methoxy-phenyl)-pyrimidin-4-ylamino]-methyl}-benzenesulfonamide,
Compound 286: rac-1-Dimethylamino-3-{4-[6-(2-methoxy-phenyl)-pyrimidin-4-ylamino]-phenoxy}-3-propan-2-ol,
Compound 287: N-[6-(4-Methoxy-phenyl)-5-methyl-pyrimidin-4-yl]-benzene-1,4-amine,
Compound 288: N-[6-(3-Amino-phenyl)-5-methyl-pyrimidin-4-yl]-benzene-1,4-amine,
Compound 289: [6-(2-Methoxy-phenyl)pyrimidin-4-yl]-piperidin-4-yl-amine,
Compound 290: 4-[6-(2-Benzyloxy-phenyl)-pyrimidin-4-ylamino]-piperidine-1-carboxylic acid tertbutyl ester,
Compound 291: Cyclohexyl-[6-(2-methoxy-phenyl)-pyrimidin-4-yl]-amine,
Compound 292: 4-{6-[2-(2-Morpholin-4-yl-ethoxy)-phenyl]-pyrimidin-4-ylamino}-benzoic acid methyl ester,
Compound 293: 2-Methoxy-4-[6-(2-methoxy-phenyl)-pyrimidin-4-ylamino]-benzoic acid methyl ester,
Compound 294: {4-[6-(2-Benzyloxy-phenyl)-pyrimidin-4-ylamino]-phenyl}-acetic acid,
Compound 295: [6-(2-Methoxy-phenyl)-pyrimidin-4-yl]-(3-nitro-phenyl)-amine,
Compound 296: {3-[6-(2-Methoxy-phenyl)-pyrimidin-4-ylamino]-phenyl}-methanol,
Compound 297: [6-(2-Benzyloxy-phenyl)-pyrimidinyl]-phenyl-amine,
Compound 298: [6-(2-Methoxy-phenyl)-pyrimidin-4-yl]-phenyl-amine,
Compound 299: (4-Fluorophenyl)-[6-(2-methoxy-phenyl)-pyrimidin-4-yl]-amine,
Compound 300: [6-(2-Methoxy-phenyl)-pyrimidin-4-yl]-(3-phenoxy-phenyl)amine,
Compound 301: [6-(2-Methoxy-phenyl)-pyrimidin-4-yl]-(3-methylsulfanyl-phenyl)-amine,
Compound 302: [6-(2-Benzyloxy-phenyl)-pyrimidin-4-yl]-piperidin-4-yl-amine,
Compound 303: 3-[6-(2-Methoxy-phenyl)-pyrimidin-4-ylamino]-phenol,
Compound 304: 1-{4-[6-(2-Methoxy-phenyl)-pyrimidin-4-ylamino]-phenyl}-ethanone,
Compound 305: 2-Chloro-4-[6-(2-methoxy-phenyl)-pyrimidin-4-ylamino}-benzoic acid,
Compound 306: {4-[6-(2-Methoxy-phenyl)-pyrimidin-4-ylamino]-butyl}-carbamic acid tert-butyl ester,
Compound 307: [6-(2-Benzyloxy-phenyl)-pyrimidin yl]-(1-methanesulfonyl-2,3-dihydro-1H-indol-6-yl)-amine,
Compound 308: 4-[6-(2-Methoxy-phenyl)-pyrimidin-4-ylamino]-piperidine-1-carboxylic acid tert-butyl ester,
Compound 309: 4-[6-(2-Amino-phenyl)-pyrimidin-4-ylamino]-benzoic acid methyl ester,
Compound 310: [6-(2-Methoxy-phenyl)-pyrimidin-4-yl]-(4-methylsulfanyl-phenyl)-amine,
Compound 311: N¹-[6-(2-Methoxy-phenyl)-pyrimidin-4-ylamino]-butane-1,4-diamine,
Compound 312: 1-{4-[6-(2-Benzyloxy-phenyl)-pyrimidin-4-ylamino]-phenoxy}-3-dimethylamino-propan-2-ol,
Compound 313: (1-Methanesulfonyl-2,3-dihydro-1H-indol-6-yl)-[6-(2-methoxy-phenyl)-pyrimidin-4-yl]-amine,
Compound 314: N-(2-Amino-cyclohexyl)-4-[6-(benzotriazol-1-yloxy)-pyrimidin-4-ylamino]-benzamide,
Compound 315: (2-{4-[6-(Benzotriazol-1-yloxy)-pyrimidin-4-ylamino]-benzoylamino)-cyclohexyl)-carbamic acid tert-butyl ester,
Compound 316: 1-{3-[6-(2-Methoxy-phenyl)-pyrimidin-4-ylamino]-phenyl}-ethanone,
Compound 317: [6-(2-Methoxy-phenyl)-pyrimidin-4-yl]-(4-piperidin-1-yl-phenyl)-amine,
Compound 318: 3-Hydroxy-4-[6-(2-methoxy-phenyl)-pyrimidin-4-ylamino]-benzoic acid methyl ester,
Compound 319: 2-Hydroxy-4-[6-(2-methoxy-phenyl)-pyrimidin-4-ylamino]-benzoic acid methyl ester,
Compound 320: 4-Amino-butane-1-sulfonic acid {5-[6-(2-methoxy-phenyl)-pyrimidin-4-ylamino]-2-methyl-phenyl}amide,
Compound 321: (3-{6-[3-(4-Amino-butane-1-sulfonylamino)-4-methyl-phenylamino]pyrimidin-4-yl}-phenyl)-carbamic acid 9H-fluoren-9-ylmethyl ester,
Compound 322: 3-Methoxy-4-[6-(2-methoxy-phenyl)-pyrimidin-4-ylamino]-benzoic acid methyl ester,
Compound 323: 4-{6-[2-(2-Pipenidin-1-yl-ethoxy)-phenyl]-pyrimidin-4-ylamino}-benzoic acid methyl ester,
Compound 324: 4-{-6-[2-(2-Dimethylamino-ethoxy)-phenyl]-pyrimidin-4-ylamino}-benzoic acid methyl ester,
Compound 325: 4-{6-[2-(2-Diisopropylamino-ethoxy)-phenyl]-pyrimidin-4-ylamino}-benzoic acid methyl ester,
Compound 326: 4-{-6-[2-(2-Diethylamino-ethoxy)-phenyl]-pyrimidin-4-ylamino}-benzoic acid methyl ester,
Compound 327: (S,S)-4-{4-[6-(2-Methoxy-phenyl)-pyrimidin-4-ylamino]-benzoylamino}-pyrrolidine-2-carboxylic acid methyl ester,
Compound 328: (S,S)-4-{4-[6-(2-Methoxy-phenyl)-pyrimidin-4-ylamino]-benzoylamino}-pyrrolidine-2-carboxylic acid,
Compound 329: (S,S)-6-[(4-{4-[6-(2-Methoxy-phenyl)-pyrimidin-4-ylamino]-benzoylamino}-pyrrolidine-2-carbonyl)-amino]-hexanoic acid,
Compound 330: N-Cyclopentyl-4-[6-(2-methoxy-phenyl)-pyrimidin-4-ylamino]-benzamide,
Compound 331: 3-[6-(2-Methoxy-phenyl)-pyrimidin-4-ylamino]-benzenesulfonamide,
Compound 332: (3-Methanesulfonyl-phenyl)-[6-(2-Methoxy-phenyl)-pyrimidin-4-yl]-amine,
Compound 333: 2-{3-[6-(2-Methoxy-phenyl)-pyrimidin-4-ylamino]-benzenesulfoyl}-ethanol,
Compound 334: N-(4,6-Dimethyl-pyrimidin-2-yl)-4-[6-(2-methoxy-phenyl)-pyrimidin-4-ylamino]-benzenesulfonamide,
Compound 335: 4-[6-(2-Methoxy-phenyl)-pyrimidin-4-ylamino]-N-thiazol-2-yl-benzenesulfonamide,
Compound 336: (1-Benzyl-piperidin-3-yl)-[6-(2-methoxy-phenyl)-pyrimidin-4-yl]-amine,
Compound 337: 3-[6-(2-Methoxy-phenyl)pyrimidin-4-ylamino]-azepan-2-one,
Compound 338: 4-[6-(2-Methoxy-phenyl)-pyrimidin-4-ylamino]-N-phenyl-benzenesulfonamide,
Compound 339: rac-[6-(2-Methoxy-phenyl)-pyrimidin-4-yl]-(1,2,3,4-tetrahydro-naphthalen-1-yl)-amine,
Compound 340: [6-(2-Methoxy-phenylpyrimidin-4-yl]-(2,2,6,6-tetramethyl-piperidin-4-yl)-amine,
Compound 341: 4-[6-(2-Methoxy-phenyl)-pyrimidin-4-ylamino]-N-methyl-benzenesulfonamide,
Compound 342: (1,1-Dioxo-1H-1λ⁶-benzo[b]thiophen-6-yl)-[6-(2-methoxy-phenylpyrimidin-4-yl]-amine,
Compound 343: N-Acetyl-4-[6-(2-methoxy-phenyl)-pyrimidin-4-ylamino]-benzenesulfonamide,
Compound 344: N-(2,6-Dimethyl-pyrimidin-4-yl)-4-[6-(2-methoxy-phenyl)-pyrimidin-4-ylamino]-benzenesulfonamide,
Compound 345: [6-(2-Methoxy-phenyl)-pyrimidin-4-yl]-[4-(piperidine-1-sulfonyl)-phenyl]-amine,
Compound 346: 3-{3-[6-(2-Methoxy-phenyl)-pyrimidin-4-ylamino]-phenoxy}-piperidine-1-carboxylic acid tert-butyl ester,
Compound 347: [6-(2-Fluoro-6-methoxy-phenyl)-pyrimidin-4-yl]-(3-methanesulfonyl-phenyl)-amine,
Compound 348: [6-(4-Fluoro-2-methoxy-phenyl)-pyrimidin-4-yl]-(3-methanesulfonyl-phenyl)-amine,
Compound 349: [6-(5-Fluoro-2-methoxy-phenyl)-pyrimidin-4-yl]-(3-methanesulfonyl-phenyl)-amine,
Compound 350: [6-(2-Methoxy-phenyl)-pyrimidin-4-yl]-pyridin-3-yl-amine,
Compound 351: 2-{4-[6-(2-Methoxy-phenyl)-pyrimidin-4-ylamino]-phenyl}-ethanol,
Compound 352: (9,9-Dioxo-9,10-dihydro-9λ⁶-thia-10-aza-phenanthren-3-yl)-[6-(2-methoxy-phenyl)-pyrimidin-4-yl]-amine,
Compound 353: [6-(2-Methoxy-phenyl)-pyrimidin-4-yl]-(1-methyl-1H-indazol-6-yl)-amine,
Compound 354: Benzo [1,2,5]thiadiazol-4-yl-[6-(2-methoxy-phenyl)-pyrimidin-4-yl]-amine,
Compound 355: Benzo [1,2,5]thiadiazol-5-yl-[6-(2-methoxy-phenyl)-pyrimidin-4-yl]-amine,
Compound 356: rac-[6-(2-Methoxy-phenyl)-pyrimidin-4-yl]-[3-(piperidin-3-yloxy)-phenyl]-amine,
Compound 357: [6-(2-Methoxy-phenyl)-pyrimidin-4-yl]-{1-[6-(2-methoxy-phenyl)-pyrimidin-4-yl]-1H-indazol-5-yl}-amine,
Compound 358: (1H-Indol-5-yl)-[6-(2-methoxy-phenyl)-pyrimidin-4-yl]-amine,
Compound 359: (3-Methanesulfinyl-phenyl)-[6-(2-methoxy-phenyl)-pyrimidin-4-yl]-amine,
Compound 360: (1H-Indazol-5-yl)[6-(2-methoxy-phenyl)-pyrimidin-4-yl]-amine,
Compound 361: 4-[6-(2-Methoxy-phenyl)-pyrimidin-4-ylamino]-thiophene-3-carboxylic acid methyl ester,
Compound 362: 4-Methanesulfonyl-benzyl-[6-(2-methoxy-phenyl)-pyrimidin-4-yl]-amine,
Compound 363: (5-Chloro-1H-indazol-3-yl)-[6-(2-methoxy-phenyl)-pyrimidin-4-yl]-amine,
Compound 364: [6-(2-Methoxy-phenyl)-pyrimidin-4-yl]-(5-methyl-isoxazol-3-yl)amine,
Compound 365: 3-[6-(2-Methoxy-phenyl)-pyrimidin-4-ylamino]-N,N-dimethyl-benzenesulfonamide,
Compound 366: N-Ethyl-3-[6-(2-methoxy-phenyl)-pyrimidin-4-ylamino]-benzenesulfonamide,
Compound 367: 3-[6-(2-Methoxy-phenyl)-pyrimidin-4-ylamino]-N-propyl-benzenesulfonamide,
Compound 368: [6-(2-Methoxy-phenyl)-pyrimidin-4-yl]-(2-methyl-1H-indol-5-yl)-amine,
Compound 369: N-(2-Methoxy-ethyl)-3-[6-(2-methoxy-phenyl)-pyrimidin-4-ylamino]-benzenesulfonamide,
Compound 370: N-tert-Butyl-3-[6-(2-methoxy-phenyl)-pyrimidin-4-ylamino]-benzenesulfonamide,
Compound 371: [6-(2-Methoxy-phenyl)-pyrimidin-4-yl]-pyridin-2-ylmethyl-amine,
Compound 372: [6-(2-Methoxy-phenyl)-pyrimidin-4-yl]-pyridin-3-ylmethyl-amine,
Compound 373: [6-(2-Methoxy-phenyl)-pyrimidin-4-yl]-pyridin ylmethyl-amine,
Compound 374: 5-[6-(2-Methoxy-phenyl)-pyrimidin-4-ylamino]-2-methyl-benzenesulfonamide,
Compound 375: N-(2-Methoxy-ethyl)-5-[6-(2-methoxy-phenyl)-pyrimidin-4-ylamino]-2-methyl-benzenesulfonamide,
Compound 376: N-(2-Hydroxy-ethyl)-5-[6-(2-methoxy-phenyl)-pyrimidin-4-ylamino]-2-methyl-benzenesulfonamide,
Compound 377: N,N-Diethyl-N-[6-(2-methoxy-phenyl)-pydrimidin-4-yl]-benzene-1,4-diamine,
Compound 378: 1-(4-Chloro-3-trifluoromethyl-phenyl)-3-{5-[6-(2-methoxy-phenyl)-pyrimidin-4-ylamino]-2-methyl-phenyl}-urea,
Compound 379: 1-Cyclohexyl-3-{5-[6-(2-methoxy-phenyl)-pyrimidin-4-ylamino]-2-methyl-phenyl}-urea,
Compound 380: [6-(2-Methoxy-phenyl)-pyrimidin-4-yl]-(4-pyrrolidin-1-yl-phenyl)-amine,
Compound 381: 4-Chloro-N-1-[6-(2-methoxy-phenyl)-pyrimidin-4-yl]-benzene-1,3-diamine,
Compound 382: 1-Isopropyl-3-{5-[6-(2-methoxy-phenyl)-pyrimidin-4-ylamino]-2-methyl-phenyl}urea,
Compound 383: 1-{5-[6-(2-Methoxy-phenyl)-pyrimidin-4-ylamino]-2-methyl-phenyl}-(2-morpholin-4-yl-ethyl)-urea,
Compound 384: 1-(2-Dimethylamino-ethyl)-3-{5-[6-(2-methoxy-phenyl)-pyrimidin-4-ylamino]-2-methyl-phenyl}-urea,
Compound 385: (4-Chloro-3-nitro-phenyl)-[6-(2-methoxy-phenyl)-pyrimidin-4-yl]-amine.

25. A method of treating inflammatory pain and/or neuropathic pain, said method comprising administering an effective amount of a composition of claim 24 to a subject in need thereof.
